SEQUENCE LISTING (16 sequences)

SEQ ID NO: 1 - protein, 503 aa, Caenorhabditis elegans
MNILLMQRIVSFILVVSQGRYFHVGELTMTMLKRPQEEESDNNATKKLKTRLTYPCILGKDKVTGKFIFPAITKDDVMNARLFLKDNDLKTFLEYFLPVEVNSIYIYFMIKLLGFDVKDKELFMALNSNITSNKERSSAELSSIHAKAEDEDELTDPLEKKHAVKLIKDLQKAINKVLSTRLRLPNFNTIDHFTATLRNAKKILVLTGAGVSTSLGIPDFRSSEGFYSKIRHLGLEDPQDVFNLDIFLQDPSVFYNIAHMVLPPENMYSPLHSFIKMLQDKGKLLRNYTQNIDNLESYAGIDPDKLVQCHGSFATASCVTCHWQIPGEKIFENIRNLELPLCPYCYQKRKQYFPMSNGNNTVQTNINFNSPILKSYGVLKPDMTFFGEALPSRFHKTIRKDILECDLLICIGTSLKVAPVSEIVNMVPSHVPQILINRDMVTHAEFDLNLLGFCDDVASLVAKKCHWDIPHKKWQDLKKIDYNCTEIDKGTYKIKKQPRKKQQ

SEQ ID NO: 2 - protein, 607 aa, Caenorhabditis elegans
MSRDSGNDSEVAVTHGEVQEITEENPEIGSMHITQETDISDAPETNTDSSRQRTESTTSVSSESWQNNDEMMSNLRRAQRLLDDGATPLQIIQQIFPDFNASRIATMSENAHFAILSDLLERAPVRQKLTNYNSLADAVELFKTKKHILVLTGAGVSVSCGIPDFRSKDGIYARLRSEFPDLPDPTAMFDIRYFRENPAPFYNFAREIFPGQFVPSVSHRFIKELETSGRLLRNYTQNIDTLEHQTGIKRVVECHGSFSKCTCTRCGQKYDGNEIREEVLAMRVAHCKRCEGVIKPNIVFFGEDLGREFHQHVTEDKHKVDLIVVIGSSLKVRPVALIPHCVDKNVPQILINRESLPHYNADIELLGNCDDIIRDICFSLGGSFTELITSYDSIMEQQGKTKSQKPSQNKRQLISQEDFLNICMKEKRNDDSSDEPTLKKPRMSVADDSMDSEKNNFQEIQKHKSEDDDDTRNSDDILKKIKHPRLLSITEMLHDNKCVAISAHQTVFPGAECSFDLETLKLVRDVHHETHCESSCGSSCSSNADSEANQLSRAQSLDDFVLSDEDRKNTIHLDLQRADSCDGDFQYELSETIDPETFSHLCEEMRI

SEQ ID NO: 3 - DNA, 4110 bp, Homo sapiens
gtcgagcgggagcagaggaggcgagggaggagggccagagaggcagttggaagatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatagtgtaataattgtgcaggtacaggaattgttccaccagcattaggaactttagcatgtcaaaatgaatgtttacttgtgaactcgatagagcaaggaaaccagaaaggtgtaatatttataggttggtaaaatagattgtttttcatggataatttttaacttcattatttctgtacttgtacaaactcaacactaacttttttttttttaaaaaaaaaaaggtactaagtatcttcaatcagctgttggtcaagactaactttcttttaaaggttcatttgtatgataaattcatatgtgtatatataattttttttgttttgtctagtgagtttcaacatttttaaagttttcaaaaagccatcggaatgttaaattaatgtaaagggaacagctaatctagaccaaagaatggtattttcacttttctttgtaacattgaatggtttgaagtactcaaaatctgttacgctaaacttttgattctttaacacaattatttttaaacactggcattttccaaaactgtggcagctaactttttaaaatctcaaatgacatgcagtgtgagtagaaggaagtcaacaatatgtggggagagcactcggttgtctttacttttaaaagtaatacttggtgctaagaatttcaggattattgtatttacgttcaaatgaagatggcttttgtacttcctgtggacatgtagtaatgtctatattggctcataaaactaacctgaaaaacaaataaatgctttggaaatgtttcagttgctttagaaacattagtgcctgcctggatccccttagttttgaaatatttgccattgttgtttaaatacctatcactgtggtagagcttgcattgatcttttccacaagtattaaactgccaaaatgtgaatatgcaaagcctttctgaatctataataatggtacttctactggggagagtgtaatattttggactgctgttttccattaatgaggagagcaacaggcccctgattatacagttccaaagtaataagatgttaattgtaattcagccagaaagtacatgtctcccattgggaggatttggtgttaaataccaaactgctagccctagtattatggagatgaacatgatgatgtaacttgtaatagcagaatagttaatgaatgaaactagttcttataatttatctttatttaaaagcttagcctgccttaaaactagagatcaactttctcagctgcaaaagcttctagtctttcaagaagttcatactttatgaaattgcacagtaagcatttatttttcagaccatttttgaacatcactcctaaattaataaagtattcctctgttgctttagtatttattacaataaaaagggtttgaaatatagctgttctttatgcataaaacacccagctaggaccattactgccagagaaaaaaatcgtattgaatggccatttccctacttataagatgtctcaatctgaatttatttggctacactaaagaatgcagtatatttagttttccatttgcatgatgtttgtgtgctatagatgatattttaaattgaaaagtttgttttaaattatttttacagtgaagactgttttcagctctttttatattgtacatagtcttttatgtaatttactggcatatgttttgtagactgtttaatgactggatatcttccttcaacttttgaaatacaaaaccagtgttttttacttgtacactgttttaaagtctattaaaattgtcatttgacttttttctgttaacttaaaaaaaaaaaaaaaaa

SEQ ID NO: 4 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 5 - DNA, 2086 bp, Homo sapiens
cattttccgggcgccctttaccaacatggctgctgacgccacgccttctgggactcgtagtccggtcctcgcgcgctttcttacctaactggggcgctctgggtgttgtacgaaagcgcgtctgcggccgcaatgtctgctgagagttgtagttctgtgccctatcacggccactcccatttctggtgccgtcacgggacagagcagtcggtgacaggacagagcagtcggtgacgggacacagtggttggtgacgggacagagcggtcggtgacagcctcaagggcttcagcaccgcgcccatggcagagccagacccctctcaccctctggagacccaggcagggaaggtgcaggaggctcaggactcagattcagactctgagggaggagccgctggtggagaagcagacatggacttcctgcggaacttattctcccagacgctcagcctgggcagccagaaggagcgtctgctggacgagctgaccttggaaggggtggcccggtacatgcagagcgaacgctgtcgcagagtcatctgtttggtgggagctggaatctccacatccgcaggcatccccgactttcgctctccatccaccggcctctatgacaacctagagaagtaccatcttccctacccagaggccatctttgagatcagctatttcaagaaacatccggaacccttcttcgccctcgccaaggaactctatcctgggcagttcaagccaaccatctgtcactacttcatgcgcctgctgaaggacaaggggctactcctgcgctgctacacgcagaacatagataccctggagcgaatagccgggctggaacaggaggacttggtggaggcgcacggcaccttctacacatcacactgcgtcagcgccagctgccggcacgaatacccgctaagctggatgaaagagaagatcttctctgaggtgacgcccaagtgtgaagactgtcagagcctggtgaagcctgatatcgtcttttttggtgagagcctcccagcgcgtttcttctcctgtatgcagtcagacttcctgaaggtggacctcctcctggtcatgggtacctccttgcaggtgcagccctttgcctccctcatcagcaaggcacccctctccacccctcgcctgctcatcaacaaggagaaagctggccagtcggaccctttcctggggatgattatgggcctcggaggaggcatggactttgactccaagaaggcctacagggacgtggcctggctgggtgaatgcgaccagggctgcctggcccttgctgagctccttggatggaagaaggagctggaggaccttgtccggagggagcacgccagcatagatgcccagtcgggggcgggggtccccaaccccagcacttcagcttcccccaagaagtccccgccacctgccaaggacgaggccaggacaacagagagggagaaaccccagtgacagctgcatctcccaggcgggatgccgagctcctcagggacagctgagccccaaccgggcctggccccctcttaaccagcagttcttgtctggggagctcagaacatcccccaatctcttacagctccctccccaaaactggggtcccagcaaccctggcccccaaccccagcaaatctctaacacctcctagaggccaaggcttaaacaggcatctctaccagccccactgtctctaaccactcctgggctaaggagtaacctccctcatctctaactgcccccacggggccagggctaccccagaacttttaactcttccaggacagggagcttcgggcccccactctgtctcctgcccccgggggcctgtggctaagtaaaccatacctaacctaccccagtgtgggtgtgggcctctgaatataacccacacccagcgtagggggagtctgagccgggagggctcccgagtctctgccttcagctcccaaagtgggtggtgggcccccttcacgtgggacccacttcccatgctggatgggcagaagacattgcttattggagacaaattaaaaacaaaaacaactaacaatccggaaaaaaaaaaaaaa

SEQ ID NO: 6 - protein, 389 aa, Homo sapiens
MAEPDPSHPLETQAGKVQEAQDSDSDSEGGAAGGEADMDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 7 - DNA, 2078 bp, Homo sapiens
agagtattcgggaggactacaactctctagccgttcccacattttccgggcgccctttaccaacatggctgctgacgccacgccttctgggactcgtagtccggtcctcgcgcgctttcttacctaactggggcgctctgggtgttgtacgaaagcgcgtctgcggccgcaatgtctgctgagagttgtagttctgtgccctatcacggccactcccatttctggtgccgtcacgggacagagcagtcggtgacaggacagagcagtcggtgacgggacacagtggttggtgacgggacagagcggtcggtgacagcctcaagggcttcagcaccgcgcccatggcagagccagaccgactcagattcagactctgagggaggagccgctggtggagaagcagacatggacttcctgcggaacttattctcccagacgctcagcctgggcagccagaaggagcgtctgctggacgagctgaccttggaaggggtggcccggtacatgcagagcgaacgctgtcgcagagtcatctgtttggtgggagctggaatctccacatccgcaggcatccccgactttcgctctccatccaccggcctctatgacaacctagagaagtaccatcttccctacccagaggccatctttgagatcagctatttcaagaaacatccggaacccttcttcgccctcgccaaggaactctatcctgggcagttcaagccaaccatctgtcactacttcatgcgcctgctgaaggacaaggggctactcctgcgctgctacacgcagaacatagataccctggagcgaatagccgggctggaacaggaggacttggtggaggcgcacggcaccttctacacatcacactgcgtcagcgccagctgccggcacgaatacccgctaagctggatgaaagagaagatcttctctgaggtgacgcccaagtgtgaagactgtcagagcctggtgaagcctgatatcgtcttttttggtgagagcctcccagcgcgtttcttctcctgtatgcagtcagacttcctgaaggtggacctcctcctggtcatgggtacctccttgcaggtgcagccctttgcctccctcatcagcaaggcacccctctccacccctcgcctgctcatcaacaaggagaaagctggccagtcggaccctttcctggggatgattatgggcctcggaggaggcatggactttgactccaagaaggcctacagggacgtggcctggctgggtgaatgcgaccagggctgcctggcccttgctgagctccttggatggaagaaggagctggaggaccttgtccggagggagcacgccagcatagatgcccagtcgggggcgggggtccccaaccccagcacttcagcttcccccaagaagtccccgccacctgccaaggacgaggccaggacaacagagagggagaaaccccagtgacagctgcatctcccaggcgggatgccgagctcctcagggacagctgagccccaaccgggcctggccccctcttaaccagcagttcttgtctggggagctcagaacatcccccaatctcttacagctccctccccaaaactggggtcccagcaaccctggcccccaaccccagcaaatctctaacacctcctagaggccaaggcttaaacaggcatctctaccagccccactgtctctaaccactcctgggctaaggagtaacctccctcatctctaactgcccccacggggccagggctaccccagaacttttaactcttccaggacagggagcttcgggcccccactctgtctcctgcccccgggggcctgtggctaagtaaaccatacctaacctaccccagtgtgggtgtgggcctctgaatataacccacacccagcgtagggggagtctgagccgggagggctcccgagtctctgccttcagctcccaaagtgggtggtgggcccccttcacgtgggacccacttcccatgctggatgggcagaagacattgcttattggagacaaattaaaaacaaaaacaactaacaatccggaaaaaaaaaaaaaa

SEQ ID NO: 8 - protein, 352 aa, Homo sapiens
MDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 9 - protein, 14 aa, Artificial sequence
  Peptide
KLGGQDIFMTEEQK

SEQ ID NO: 10 - protein, 2016 aa, Homo sapiens [sequence omitted]

SEQ ID NO: 11 - DNA, 8504 bp, Homo sapiens
agacggcggcggcgcccgtaggatgcagggatcgctcccccggggccgctgagcctgcgcccagtgccccgagccccgcgccgagccgagtccgcgccaagcagcagccgcccaccccggggcccggccgggggaccagcagcttccccacaggcaacgtgaggagagcctgtgcccagaagcaggatgagaagatggcaaacttcctattacctcggggcaccagcagcttccgcaggttcacacgggagtccctggcagccatcgagaagcgcatggcagagaagcaagcccgcggctcaaccaccttgcaggagagccgagaggggctgcccgaggaggaggctccccggccccagctggacctgcaggcctccaaaaagctgccagatctctatggcaatccaccccaagagctcatcggagagcccctggaggacctggaccccttctatagcacccaaaagactttcatcgtactgaataaaggcaagaccatcttccggttcagtgccaccaacgccttgtatgtcctcagtcccttccaccccatccggagagcggctgtgaagattctggttcactcgctcttcaacatgctcatcatgtgcaccatcctcaccaactgcgtgttcatggcccagcacgaccctccaccctggaccaagtatgtcgagtacaccttcaccgccatttacacctttgagtctctggtcaagattctggctcgaggcttctgcctgcacgcgttcactttccttcgggacccatggaactggctggactttagtgtgattatcatggcatacacaactgaatttgtggacctgggcaatgtctcagccttacgcaccttccgagtcctccgggccctgaaaactatatcagtcatttcagggctgaagaccatcgtgggggccctgatccagtctgtgaagaagctggctgatgtgatggtcctcacagtcttctgcctcagcgtctttgccctcatcggcctgcagctcttcatgggcaacctaaggcacaagtgcgtgcgcaacttcacagcgctcaacggcaccaacggctccgtggaggccgacggcttggtctgggaatccctggacctttacctcagtgatccagaaaattacctgctcaagaacggcacctctgatgtgttactgtgtgggaacagctctgacgctgggacatgtccggagggctaccggtgcctaaaggcaggcgagaaccccgaccacggctacaccagcttcgattcctttgcctgggcctttcttgcactcttccgcctgatgacgcaggactgctgggagcgcctctatcagcagaccctcaggtccgcagggaagatctacatgatcttcttcatgcttgtcatcttcctggggtccttctacctggtgaacctgatcctggccgtggtcgcaatggcctatgaggagcaaaaccaagccaccatcgctgagaccgaggagaaggaaaagcgcttccaggaggccatggaaatgctcaagaaagaacacgaggccctcaccatcaggggtgtggataccgtgtcccgtagctccttggagatgtcccctttggccccagtaaacagccatgagagaagaagcaagaggagaaaacggatgtcttcaggaactgaggagtgtggggaggacaggctccccaagtctgactcagaagatggtcccagagcaatgaatcatctcagcctcacccgtggcctcagcaggacttctatgaagccacgttccagccgcgggagcattttcacctttcgcaggcgagacctgggttctgaagcagattttgcagatgatgaaaacagcacagcgggggagagcgagagccaccacacatcactgctggtgccctggcccctgcgccggaccagtgcccagggacagcccagtcccggaacctcggctcctggccacgccctccatggcaaaaagaacagcactgtggactgcaatggggtggtctcattactgggggcaggcgacccagaggccacatccccaggaagccacctcctccgccctgtgatgctagagcacccgccagacacgaccacgccatcggaggagccaggcgggccccagatgctgacctcccaggctccgtgtgtagatggcttcgaggagccaggagcacggcagcgggccctcagcgcagtcagcgtcctcaccagcgcactggaagagttagaggagtctcgccacaagtgtccaccatgctggaaccgtctcgcccagcgctacctgatctgggagtgctgcccgctgtggatgtccatcaagcagggagtgaagttggtggtcatggacccgtttactgacctcaccatcactatgtgcatcgtactcaacacactcttcatggcgctggagcactacaacatgacaagtgaattcgaggagatgctgcaggtcggaaacctggtcttcacagggattttcacagcagagatgaccttcaagatcattgccctcgacccctactactacttccaacagggctggaacatcttcgacagcatcatcgtcatccttagcctcatggagctgggcctgtcccgcatgagcaacttgtcggtgctgcgctccttccgcctgctgcgggtcttcaagctggccaaatcatggcccaccctgaacacactcatcaagatcatcgggaactcagtgggggcactggggaacctgacactggtgctagccatcatcgtgttcatctttgctgtggtgggcatgcagctctttggcaagaactactcggagctgagggacagcgactcaggcctgctgcctcgctggcacatgatggacttctttcatgccttcctcatcatcttccgcatcctctgtggagagtggatcgagaccatgtgggactgcatggaggtgtcggggcagtcattatgcctgctggtcttcttgcttgttatggtcattggcaaccttgtggtcctgaatctcttcctggccttgctgctcagctccttcagtgcagacaacctcacagcccctgatgaggacagagagatgaacaacctccagctggccctggcccgcatccagaggggcctgcgctttgtcaagcggaccacctgggatttctgctgtggtctcctgcggcagcggcctcagaagcccgcagcccttgccgcccagggccagctgcccagctgcattgccaccccctactccccgccacccccagagacggagaaggtgcctcccacccgcaaggaaacacggtttgaggaaggcgagcaaccaggccagggcacccccggggatccagagcccgtgtgtgtgcccatcgctgtggccgagtcagacacagatgaccaagaagaagatgaggagaacagcctgggcacggaggaggagtccagcaagcagcaggaatcccagcctgtgtccggtggcccagaggcccctccggattccaggacctggagccaggtgtcagcgactgcctcctctgaggccgaggccagtgcatctcaggccgactggcggcagcagtggaaagcggaaccccaggccccagggtgcggtgagaccccagaggacagttgctccgagggcagcacagcagacatgaccaacaccgctgagctcctggagcagatccctgacctcggccaggatgtcaaggacccagaggactgcttcactgaaggctgtgtccggcgctgtccctgctgtgcggtggacaccacacaggccccagggaaggtctggtggcggttgcgcaagacctgctaccacatcgtggagcacagctggttcgagacattcatcatcttcatgatcctactcagcagtggagcgctggccttcgaggacatctacctagaggagcggaagaccatcaaggttctgcttgagtatgccgacaagatgttcacatatgtcttcgtgctggagatgctgctcaagtgggtggcctacggcttcaagaagtacttcaccaatgcctggtgctggctcgacttcctcatcgtagacgtctctctggtcagcctggtggccaacaccctgggctttgccgagatgggccccatcaagtcactgcggacgctgcgtgcactccgtcctctgagagctctgtcacgatttgagggcatgagggtggtggtcaatgccctggtgggcgccatcccgtccatcatgaacgtcctcctcgtctgcctcatcttctggctcatcttcagcatcatgggcgtgaacctctttgcggggaagtttgggaggtgcatcaaccagacagagggagacttgcctttgaactacaccatcgtgaacaacaagagccagtgtgagtccttgaacttgaccggagaattgtactggaccaaggtgaaagtcaactttgacaacgtgggggccgggtacctggcccttctgcaggtggcaacatttaaaggctggatggacattatgtatgcagctgtggactccagggggtatgaagagcagcctcagtgggaatacaacctctacatgtacatctattttgtcattttcatcatctttgggtctttcttcaccctgaacctctttattggtgtcatcattgacaacttcaaccaacagaagaaaaagttagggggccaggacatcttcatgacagaggagcagaagaagtactacaatgccatgaagaagctgggctccaagaagccccagaagcccatcccacggcccctgaacaagtaccagggcttcatattcgacattgtgaccaagcaggcctttgacgtcaccatcatgtttctgatctgcttgaatatggtgaccatgatggtggagacagatgaccaaagtcctgagaaaatcaacatcttggccaagatcaacctgctctttgtggccatcttcacaggcgagtgtattgtcaagctggctgccctgcgccactactacttcaccaacagctggaatatcttcgacttcgtggttgtcatcctctccatcgtgggcactgtgctctcggacatcatccagaagtacttcttctccccgacgctcttccgagtcatccgcctggcccgaataggccgcatcctcagactgatccgaggggccaaggggatccgcacgctgctctttgccctcatgatgtccctgcctgccctcttcaacatcgggctgctgctcttcctcgtcatgttcatctactccatctttggcatggccaacttcgcttatgtcaagtgggaggctggcatcgacgacatgttcaacttccagaccttcgccaacagcatgctgtgcctcttccagatcaccacgtcggccggctgggatggcctcctcagccccatcctcaacactgggccgccctactgcgaccccactctgcccaacagcaatggctctcggggggactgcgggagcccagccgtgggcatcctcttcttcaccacctacatcatcatctccttcctcatcgtggtcaacatgtacattgccatcatcctggagaacttcagcgtggccacggaggagagcaccgagcccctgagtgaggacgacttcgatatgttctatgagatctgggagaaatttgacccagaggccactcagtttattgagtattcggtcctgtctgactttgccgatgccctgtctgagccactccgtatcgccaagcccaaccagataagcctcatcaacatggacctgcccatggtgagtggggaccgcatccattgcatggacattctctttgccttcaccaaaagggtcctgggggagtctggggagatggacgccctgaagatccagatggaggagaagttcatggcagccaacccatccaagatctcctacgagcccatcaccaccacactccggcgcaagcacgaagaggtgtcggccatggttatccagagagccttccgcaggcacctgctgcaacgctctttgaagcatgcctccttcctcttccgtcagcaggcgggcagcggcctctccgaagaggatgcccctgagcgagagggcctcatcgcctacgtgatgagtgagaacttctcccgaccccttggcccaccctccagctcctccatctcctccacttccttcccaccctcctatgacagtgtcactagagccaccagcgataacctccaggtgcgggggtctgactacagccacagtgaagatctcgccgacttccccccttctccggacagggaccgtgagtccatcgtgtgagcctcggcctggctggccaggacacactgaaaagcagcctttttcaccatggcaaacctaaatgcagtcagtcacaaaccagcctggggccttcctggctttgggagtaagaaatgggcctcagccccgcggatcaaccaggcagagttctgtggcgccgcgtggacagccggagcagttggcctgtgcttggaggcctcagatagacctgtgacctggtctggtcaggcaatgccctgcggctctggaaagcaacttcatcccagctgctgaggcgaaatataaaactgagactgtatatgttgtgaatgggctttcataaatttattatatttgatatttttttacttgagcaaagaactaaggatttttccatggacatgggcagcaattcacgctgtctcttcttaaccctgaacaagagtgtctatggagcagccggaagtctgttctcaaagcagaagtggaatccagtgtggctcccacaggtcttcactgcccaggggtcgaatggggtccccctcccacttgacctgagatgctgggagggctgaacccccactcacacaagcacacacacacagtcctcacacacggaggccagacacaggccgtgggacccaggctcccagcctaagggagacaggcctttccctgccggccccccaaggatggggttcttgtccacggggctcactctggccccctattgtctccaaggtcccattttccccctgtgttttcacgcaggtcatattgtcagtcctacaaaaataaaaggcttccagaggagagtggcctgggtcccagggctggccctaggcactgatagttgccttttcttcccctcctgtaagagtattaacaaaaccaaaggacacaagggtgcaagccccattcacggcctggcatgcagcttgtccttgctcctggaacctggcaggccctgcccagccagccatcggaagagagggctgagccatgggggtttggggctaagaagttcaccagccctgagccatggcggcccctcagcctgcctgaagagaggaaactggcgatctcccagggctctctggaccatacgcggaggagttttctgtgtggtctccagctcctctccagacacagagacatgggagtggggagcggagcttggccctgcgccctgtgcagggaaagggatggtcaggcccagttctcgtgcccttagaggggaatgaaccatggcacctttgagagagggggcactgtggtcaggcccagcctctctggctcagcccgggatcctgatggcacccacacagaggacctctttggggcaagatccaggtggtcccataggtcttgtgaaaaggctttttcagggaaaaatattttactagtccaatcacccccaggacctcttcagctgctgacaatcctatttagcatatgcaaatcttttaacatagagaactgtcaccctgaggtaacagggtcaactggcgaagcctgagcaggcaggggcttggctgccccattccagctctcccatggagcccctccaccgggcgcatgcctcccaggccacctcagtctcacctgccggctctgggctggctgctcctaacctacctcgccgagctgtcggagggctggacatttgtggcagtgctgaagggggcattgccggcgagtaaagtattatgtttcttcttgtcaccccagttcccttggtggcaaccccagacccaacccatgcccctgacagatctagttctcttctcctgtgttccctttgagtccagtgtgggacacggtttaactgtcccagcgacatttctccaagtggaaatcctatttttgtagatctccatgctttgctctcaaggcttggagaggtatgtgcccctcctgggtgctcaccgcctgctacacaggcaggaatgcggttgggaggcaggtcgggctgccagcccagctggccggaaggagactgtggtttttgtgtgtgtggacagcccgggagctttgagacaggtgcctggggctggctgcagacggtgtggttgggggtgggaggtgagctagacccaacccttagcttttagcctggctgtcacctttttaatttccagaactgcacaatgaccagcaggagggaaggacagacatcaagtgccagatgttgtctgaactaatcgagcacttctcaccaaacttcatgtataaataaaatacatatttttaaaacaaaccaataaatggcttacatga

SEQ ID NO: 12 - protein, 2015 aa, Homo sapiens [sequence omitted]

SEQ ID NO: 13 - protein, 2016 aa, Homo sapiens
MANFLLPRGTSSFRRFTRESLAAIEKRMAEKQARGSTTLQESREGLPEEEAPRPQLDLQASKKLPDLYGNPPQELIGEPLEDLDPFYSTQKTFIVLNKGKTIFRFSATNALYVLSPFHPIRRAAVKILVHSLFNMLIMCTILTNCVFMAQHDPPPWTKYVEYTFTAIYTFESLVKILARGFCLHAFTFLRDPWNWLDFSVIIMAYVSENIKLGNLSALRTFRVLRALKTISVIPGLKTIVGALIQSVKKLADVMVLTVFCLSVFALIGLQLFMGNLRHKCVRNFTALNGTNGSVEADGLVWESLDLYLSDPENYLLKNGTSDVLLCGNSSDAGTCPEGYRCLKAGENPDHGYTSFDSFAWAFLALFRLMTQDCWERLYQQTLRSAGKIYMIFFMLVIFLGSFYLVNLILAVVAMAYEEQNQATIAETEEKEKRFQEAMEMLKKEHEALTIRGVDTVSRSSLEMSPLAPVNSHERRSKRRKRMSSGTEECGEDRLPKSDSEDGPRAMNHLSLTRGLSRTSMKPRSSRGSIFTFRRRDLGSEADFADDENSTAGESESHHTSLLVPWPLRRTSAQGQPSPGTSAPGHALHGKKNSTVDCNGVVSLLGAGDPEATSPGSHLLRPVMLEHPPDTTTPSEEPGGPQMLTSQAPCVDGFEEPGARQRALSAVSVLTSALEELEESRHKCPPCWNRLAQRYLIWECCPLWMSIKQGVKLVVMDPFTDLTITMCIVLNTLFMALEHYNMTSEFEEMLQVGNLVFTGIFTAEMTFKIIALDPYYYFQQGWNIFDSIIVILSLMELGLSRMSNLSVLRSFRLLRVFKLAKSWPTLNTLIKIIGNSVGALGNLTLVLAIIVFIFAVVGMQLFGKNYSELRDSDSGLLPRWHMMDFFHAFLIIFRILCGEWIETMWDCMEVSGQSLCLLVFLLVMVIGNLVVLNLFLALLLSSFSADNLTAPDEDREMNNLQLALARIQRGLRFVKRTTWDFCCGLLRQRPQKPAALAAQGQLPSCIATPYSPPPPETEKVPPTRKETRFEEGEQPGQGTPGDPEPVCVPIAVAESDTDDQEEDEENSLGTEEESSKQQESQPVSGGPEAPPDSRTWSQVSATASSEAEASASQADWRQQWKAEPQAPGCGETPEDSCSEGSTADMTNTAELLEQIPDLGQDVKDPEDCFTEGCVRRCPCCAVDTTQAPGKVWWRLRKTCYHIVEHSWFETFIIFMILLSSGALAFEDIYLEERKTIKVLLEYADKMFTYVFVLEMLLKWVAYGFKKYFTNAWCWLDFLIVDVSLVSLVANTLGFAEMGPIKSLRTLRALRPLRALSRFEGMRVVVNALVGAIPSIMNVLLVCLIFWLIFSIMGVNLFAGKFGRCINQTEGDLPLNYTIVNNKSQCESLNLTGELYWTKVKVNFDNVGAGYLALLQVATFKGWMDIMYAAVDSRGYEEQPQWEYNLYMYIYFVIFIIFGSFFTLNLFIGVIIDNFNQQKKKLGGQDIFMTEEQKKYYNAMKKLGSKKPQKPIPRPLNKYQGFIFDIVTKQAFDVTIMFLICLNMVTMMVETDDQSPEKINILAKINLLFVAIFTGECIVKLAALRHYYFTNSWNIFDFVVVILSIVGTVLSDIIQKYFFSPTLFRVIRLARIGRILRLIRGAKGIRTLLFALMMSLPALFNIGLLLFLVMFIYSIFGMANFAYVKWEAGIDDMFNFQTFANSMLCLFQITTSAGWDGLLSPILNTGPPYCDPTLPNSNGSRGDCGSPAVGILFFTTYIIISFLIVVNMYIAIILENFSVATEESTEPLSEDDFDMFYEIWEKFDPEATQFIEYSVLSDFADALSEPLRIAKPNQISLINMDLPMVSGDRIHCMDILFAFTKRVLGESGEMDALKIQMEEKFMAANPSKISYEPITTTLRRKHEEVSAMVIQRAFRRHLLQRSLKHASFLFRQQAGSGLSEEDAPEREGLIAYVMSENFSRPLGPPSSSSISSTSFPPSYDSVTRATSDNLQVRGSDYSHSEDLADFPPSPDRDRESIV

SEQ ID NO: 14 - protein, 1998 aa, Homo sapiens
MANFLLPRGTSSFRRFTRESLAAIEKRMAEKQARGSTTLQESREGLPEEEAPRPQLDLQASKKLPDLYGNPPQELIGEPLEDLDPFYSTQKTFIVLNKGKTIFRFSATNALYVLSPFHPIRRAAVKILVHSLFNMLIMCTILTNCVFMAQHDPPPWTKYVEYTFTAIYTFESLVKILARGFCLHAFTFLRDPWNWLDFSVIIMAYVSENIKLGNLSALRTFRVLRALKTISVIPGLKTIVGALIQSVKKLADVMVLTVFCLSVFALIGLQLFMGNLRHKCVRNFTALNGTNGSVEADGLVWESLDLYLSDPENYLLKNGTSDVLLCGNSSDAGTCPEGYRCLKAGENPDHGYTSFDSFAWAFLALFRLMTQDCWERLYQQTLRSAGKIYMIFFMLVIFLGSFYLVNLILAVVAMAYEEQNQATIAETEEKEKRFQEAMEMLKKEHEALTIRGVDTVSRSSLEMSPLAPVNSHERRSKRRKRMSSGTEECGEDRLPKSDSEDGPRAMNHLSLTRGLSRTSMKPRSSRGSIFTFRRRDLGSEADFADDENSTAGESESHHTSLLVPWPLRRTSAQGQPSPGTSAPGHALHGKKNSTVDCNGVVSLLGAGDPEATSPGSHLLRPVMLEHPPDTTTPSEEPGGPQMLTSQAPCVDGFEEPGARQRALSAVSVLTSALEELEESRHKCPPCWNRLAQRYLIWECCPLWMSIKQGVKLVVMDPFTDLTITMCIVLNTLFMALEHYNMTSEFEEMLQVGNLVFTGIFTAEMTFKIIALDPYYYFQQGWNIFDSIIVILSLMELGLSRMSNLSVLRSFRLLRVFKLAKSWPTLNTLIKIIGNSVGALGNLTLVLAIIVFIFAVVGMQLFGKNYSELRDSDSGLLPRWHMMDFFHAFLIIFRILCGEWIETMWDCMEVSGQSLCLLVFLLVMVIGNLVVLNLFLALLLSSFSADNLTAPDEDREMNNLQLALARIQRGLRFVKRTTWDFCCGLLRQRPQKPAALAAQGQLPSCIATPYSPPPPETEKVPPTRKETRFEEGEQPGQGTPGDPEPVCVPIAVAESDTDDQEEDEENSLGTEEESSKQQESQPVSGGPEAPPDSRTWSQVSATASSEAEASASQADWRQQWKAEPQAPGCGETPEDSCSEGSTADMTNTAELLEQIPDLGQDVKDPEDCFTEGCVRRCPCCAVDTTQAPGKVWWRLRKTCYHIVEHSWFETFIIFMILLSSGALAFEDIYLEERKTIKVLLEYADKMFTYVFVLEMLLKWVAYGFKKYFTNAWCWLDFLIVDVSLVSLVANTLGFAEMGPIKSLRTLRALRPLRALSRFEGMRVVVNALVGAIPSIMNVLLVCLIFWLIFSIMGVNLFAGKFGRCINQTEGDLPLNYTIVNNKSQCESLNLTGELYWTKVKVNFDNVGAGYLALLQVYEEQPQWEYNLYMYIYFVIFIIFGSFFTLNLFIGVIIDNFNQQKKKLGGQDIFMTEEQKKYYNAMKKLGSKKPQKPIPRPLNKYQGFIFDIVTKQAFDVTIMFLICLNMVTMMVETDDQSPEKINILAKINLLFVAIFTGECIVKLAALRHYYFTNSWNIFDFVVVILSIVGTVLSDIIQKYFFSPTLFRVIRLARIGRILRLIRGAKGIRTLLFALMMSLPALFNIGLLLFLVMFIYSIFGMANFAYVKWEAGIDDMFNFQTFANSMLCLFQITTSAGWDGLLSPILNTGPPYCDPTLPNSNGSRGDCGSPAVGILFFTTYIIISFLIVVNMYIAIILENFSVATEESTEPLSEDDFDMFYEIWEKFDPEATQFIEYSVLSDFADALSEPLRIAKPNQISLINMDLPMVSGDRIHCMDILFAFTKRVLGESGEMDALKIQMEEKFMAANPSKISYEPITTTLRRKHEEVSAMVIQRAFRRHLLQRSLKHASFLFRQQAGSGLSEEDAPEREGLIAYVMSENFSRPLGPPSSSSISSTSFPPSYDSVTRATSDNLQVRGSDYSHSEDLADFPPSPDRDRESIV

SEQ ID NO: 15 - protein, 1983 aa, Homo sapiens
MANFLLPRGTSSFRRFTRESLAAIEKRMAEKQARGSTTLQESREGLPEEEAPRPQLDLQASKKLPDLYGNPPQELIGEPLEDLDPFYSTQKTFIVLNKGKTIFRFSATNALYVLSPFHPIRRAAVKILVHSLFNMLIMCTILTNCVFMAQHDPPPWTKYVEYTFTAIYTFESLVKILARGFCLHAFTFLRDPWNWLDFSVIIMAYVSENIKLGNLSALRTFRVLRALKTISVIPGLKTIVGALIQSVKKLADVMVLTVFCLSVFALIGLQLFMGNLRHKCVRNFTALNGTNGSVEADGLVWESLDLYLSDPENYLLKNGTSDVLLCGNSSDAGTCPEGYRCLKAGENPDHGYTSFDSFAWAFLALFRLMTQDCWERLYQQTLRSAGKIYMIFFMLVIFLGSFYLVNLILAVVAMAYEEQNQATIAETEEKEKRFQEAMEMLKKEHEALTIRGVDTVSRSSLEMSPLAPVNSHERRSKRRKRMSSGTEECGEDRLPKSDSEDGPRAMNHLSLTRGLSRTSMKPRSSRGSIFTFRRRDLGSEADFADDENSTAGESESHHTSLLVPWPLRRTSAQGQPSPGTSAPGHALHGKKNSTVDCNGVVSLLGAGDPEATSPGSHLLRPVMLEHPPDTTTPSEEPGGPQMLTSQAPCVDGFEEPGARQRALSAVSVLTSALEELEESRHKCPPCWNRLAQRYLIWECCPLWMSIKQGVKLVVMDPFTDLTITMCIVLNTLFMALEHYNMTSEFEEMLQVGNLVFTGIFTAEMTFKIIALDPYYYFQQGWNIFDSIIVILSLMELGLSRMSNLSVLRSFRLLRVFKLAKSWPTLNTLIKIIGNSVGALGNLTLVLAIIVFIFAVVGMQLFGKNYSELRDSDSGLLPRWHMMDFFHAFLIIFRILCGEWIETMWDCMEVSGQSLCLLVFLLVMVIGNLVVLNLFLALLLSSFSADNLTAPDEDREMNNLQLALARIQRGLRFVKRTTWDFCCGLLRQRPQKPAALAAQGQLPSCIATPYSPPPPETEKVPPTRKETRFEEGEQPGQGTPGDPEPVCVPIAVAESDTDDQEEDEENSLGTEEESSKQESQPVSGGPEAPPDSRTWSQVSATASSEAEASASQADWRQQWKAEPQAPGCGETPEDSCSEGSTADMTNTAELLEQIPDLGQDVKDPEDCFTEGCVRRCPCCAVDTTQAPGKVWWRLRKTCYHIVEHSWFETFIIFMILLSSGALAFEDIYLEERKTIKVLLEYADKMFTYVFVLEMLLKWVAYGFKKYFTNAWCWLDFLIVDVSLVSLVANTLGFAEMGPIKSLRTLRALRPLRALSRFEGMRVVVNALVGAIPSIMNVLLVCLIFWLIFSIMGVNLFAGKFGRCINQTEGDLPLNYTIVNNKSQCESLNLTGELYWTKVKVNFDNVGAGYLALLQVATFKGWMDIMYAAVDSRGYEEQPQWEYNLYMYIYFVIFIIFGSFFTLNLFIGVIIDNFNQQKKKLGGQDIFMTEEQKKYYNAMKKLGSKKPQKPIPRPLNKYQGFIFDIVTKQAFDVTIMFLICLNMVTMMVETDDQSPEKINILAKINLLFVAIFTGTVLSDIIQKYFFSPTLFRVIRLARIGRILRLIRGAKGIRTLLFALMMSLPALFNIGLLLFLVMFIYSIFGMANFAYVKWEAGIDDMFNFQTFANSMLCLFQITTSAGWDGLLSPILNTGPPYCDPTLPNSNGSRGDCGSPAVGILFFTTYIIISFLIVVNMYIAIILENFSVATEESTEPLSEDDFDMFYEIWEKFDPEATQFIEYSVLSDFADALSEPLRIAKPNQISLINMDLPMVSGDRIHCMDILFAFTKRVLGESGEMDALKIQMEEKFMAANPSKISYEPITTTLRRKHEEVSAMVIQRAFRRHLLQRSLKHASFLFRQQAGSGLSEEDAPEREGLIAYVMSENFSRPLGPPSSSSISSTSFPPSYDSVTRATSDNLQVRGSDYSHSEDLADFPPSPDRDRESIV

SEQ ID NO: 16 - protein, 1962 aa, Homo sapiens
MANFLLPRGTSSFRRFTRESLAAIEKRMAEKQARGSTTLQESREGLPEEEAPRPQLDLQASKKLPDLYGNPPQELIGEPLEDLDPFYSTQKTFIVLNKGKTIFRFSATNALYVLSPFHPIRRAAVKILVHSLFNMLIMCTILTNCVFMAQHDPPPWTKYVEYTFTAIYTFESLVKILARGFCLHAFTFLRDPWNWLDFSVIIMAYVSENIKLGNLSALRTFRVLRALKTISVIPGLKTIVGALIQSVKKLADVMVLTVFCLSVFALIGLQLFMGNLRHKCVRNFTALNGTNGSVEADGLVWESLDLYLSDPENYLLKNGTSDVLLCGNSSDAGTCPEGYRCLKAGENPDHGYTSFDSFAWAFLALFRLMTQDCWERLYQQTLRSAGKIYMIFFMLVIFLGSFYLVNLILAVVAMAYEEQNQATIAETEEKEKRFQEAMEMLKKEHEALTIRGVDTVSRSSLEMSPLAPVNSHERRSKRRKRMSSGTEECGEDRLPKSDSEDGPRAMNHLSLTRGLSRTSMKPRSSRGSIFTFRRRDLGSEADFADDENSTAGESESHHTSLLVPWPLRRTSAQGQPSPGTSAPGHALHGKKNSTVDCNGVVSLLGAGDPEATSPGSHLLRPVMLEHPPDTTTPSEEPGGPQMLTSQAPCVDGFEEPGARQRALSAVSVLTSALEELEESRHKCPPCWNRLAQRYLIWECCPLWMSIKQGVKLVVMDPFTDLTITMCIVLNTLFMALEHYNMTSEFEEMLQVGNLVFTGIFTAEMTFKIIALDPYYYFQQGWNIFDSIIVILSLMELGLSRMSNLSVLRSFRLLRVFKLAKSWPTLNTLIKIIGNSVGALGNLTLVLAIIVFIFAVVGMQLFGKNYSELRDSDSGLLPRWHMMDFFHAFLIIFRILCGEWIETMWDCMEVSGQSLCLLVFLLVMVIGNLVVLNLFLALLLSSFSADNLTAPDEDREMNNLQLALARIQRGLRFVKRTTWDFCCGLLRQRPQKPAALAAQGQLPSCIATPYSPPPPETEKVPPTRKETRFEEGEQPGQGTPGDPEPVCVPIAVAESDTDDQEEDEENSLGTEEESSKQTPEDSCSEGSTADMTNTAELLEQIPDLGQDVKDPEDCFTEGCVRRCPCCAVDTTQAPGKVWWRLRKTCYHIVEHSWFETFIIFMILLSSGALAFEDIYLEERKTIKVLLEYADKMFTYVFVLEMLLKWVAYGFKKYFTNAWCWLDFLIVDVSLVSLVANTLGFAEMGPIKSLRTLRALRPLRALSRFEGMRVVVNALVGAIPSIMNVLLVCLIFWLIFSIMGVNLFAGKFGRCINQTEGDLPLNYTIVNNKSQCESLNLTGELYWTKVKVNFDNVGAGYLALLQVATFKGWMDIMYAAVDSRGYEEQPQWEYNLYMYIYFVIFIIFGSFFTLNLFIGVIIDNFNQQKKKLGGQDIFMTEEQKKYYNAMKKLGSKKPQKPIPRPLNKYQGFIFDIVTKQAFDVTIMFLICLNMVTMMVETDDQSPEKINILAKINLLFVAIFTGECIVKLAALRHYYFTNSWNIFDFVVVILSIVGTVLSDIIQKYFFSPTLFRVIRLARIGRILRLIRGAKGIRTLLFALMMSLPALFNIGLLLFLVMFIYSIFGMANFAYVKWEAGIDDMFNFQTFANSMLCLFQITTSAGWDGLLSPILNTGPPYCDPTLPNSNGSRGDCGSPAVGILFFTTYIIISFLIVVNMYIAIILENFSVATEESTEPLSEDDFDMFYEIWEKFDPEATQFIEYSVLSDFADALSEPLRIAKPNQISLINMDLPMVSGDRIHCMDILFAFTKRVLGESGEMDALKIQMEEKFMAANPSKISYEPITTTLRRKHEEVSAMVIQRAFRRHLLQRSLKHASFLFRQQAGSGLSEEDAPEREGLIAYVMSENFSRPLGPPSSSSISSTSFPPSYDSVTRATSDNLQVRGSDYSHSEDLADFPPSPDRDRESIV

(19) United States
(12) Patent Application Publication
(10) Pub. No.: US 2015/0133527 A1
(43) Pub. Date: May 14, 2015

(54) REGULATION OF CARDIAC SODIUM CHANNELS BY SIRT1 AND SIRT1 ACTIVATORS

(71) Applicant: University of Pittsburgh - Of the Commonwealth System of Higher Education, Pittsburgh, PA (US)

(72) Inventors: Barry London, Iowa City, IA (US); Kaikobad J. Irani, Iowa City, IA (US)

(21) Appl. No.: 14/398,723

(22) PCT Filed: May 6, 2013
(86) PCT No.: PCT/US2013/039747

§ 371 Date: Nov. 3, 2014

Related U.S. Application Data
(60) Provisional application No. 61/643,095, filed on May 4, 2012.

(51) Int. Cl. A61K 31/5513; A61K 31/713; A61K 31/196; A61K 31/5377; A61K 31/341; A61K 31/437; A61K 31/5375; A61K 31/245; A61K 31/403

(52) U.S. Cl. CPC A61K 31/5513; A61K 31/245; A61K 31/713; A61K 31/196; A61K 31/403; A61K 31/341; A61K 31/437; A61K 31/5375; A61K 31/5377
USPC 514/44 R; 514/548; 514/563; 514/411; 514/471; 514/302; 514/239.5; 514/234.2; 514/218

(57) ABSTRACT

Methods are provided herein for treating cardiac arrhythmias, such as for treating an arrhythmia syndrome, for example Brugada syndrome, in a subject. In some embodiments, the methods include selecting a subject with Brugada syndrome and administering to the subject an effective amount of an agent that increases the expression or activity of SIRT1 in the subject. In some embodiments, the agent increases Nav1.5 activation. In some embodiments, the agent increases the expression or activity of SRIT1 and increases Nav1.5 activation.

### RELATED APPLICATIONS

This application claims the benefit of U.S. Provisional Application No. 61/643,095, filed May 4, 2012, which is incorporated by reference herein in its entirety.

### ACKNOWLEDGMENT OF GOVERNMENT SUPPORT

This invention was made with government support under Grant Number NIH NHLBI RO1 HL062300 awarded by the National Institutes of Health. The government has certain rights in the invention.

### FIELD

This application relates to the field of cardiac arrhythmia, specifically to methods and agents for use in the treatment of arrhythmia syndromes, such as the treatment of Brugada syndrome.

### BACKGROUND

Arrhythmias and the sudden death that they cause remain a major health problem today. Brugada Syndrome is a congenital arrhythmia syndrome which manifests as syncope, ventricular fibrillation, and sudden cardiac death in patients without overt structural heart disease in association with surface electrocardiogram (ECG) abnormalities. Brugada Syndrome is inherited in an autosomal dominant manner, and diagnosed predominantly in men. Sudden death is common and may be the first manifestation of this disease. Except for the placement of prophylactic cardiac defibrillators, there are no effective and acceptable therapies for Brugada Syndrome.

The cardiac Na⁺ channel Nav1.5 (Nav1.5) and the inward depolarizing Na⁺ current (I_{Na}) play a critical role in regulating the action potential of myocytes in the atrium and ventricle, and in maintaining rapid conduction velocity throughout the heart. Loss of function mutations in Nav1.5 that decrease I_{Na} are associated with cardiac arrhythmias, and cause ˜20% of cases of Brugada syndrome.

Silencing Information Regulators (SIR) are a family of histone deacetylases (HDACs), first identified in yeast, that are collectively known as SIRTUIN proteins. SIRT1 (SIRTUIN1) is the closest mammalian homologue of yeast Sir2, and is a ubiquitously expressed mammalian deacetylase that targets specific acetylated lysine residues on histones and non-histone proteins.

### SUMMARY

It is disclosed herein that agents that increase the expression and/or activity of SIRT1, including SIRT1 itself, can be used in therapies for arrhythmia syndromes, such as Brugada syndrome. In view of this surprising finding, methods are provided herein for treating arrhythmia syndromes, such as for treating Brugada syndrome, in a subject.

Some embodiments include a method for treating Brugada syndrome in a subject, including selecting a subject with Brugada syndrome, and administering to the subject an effective amount of an agent that increases the expression or activity of SIRT1 in the subject, thereby treating Brugada syndrome in the subject. In several such embodiments, the agent that increases the expression or activity of SRIT1 is a SIRT1 activator.

For example, the agent can include Structure I:

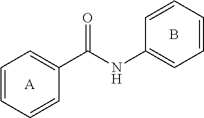

or a salt thereof, wherein, Ring A is optionally substituted, fused to another ring or both; and Ring B is substituted with at least one carboxy, substituted or unsubstituted arylcarboxamine, substituted or unsubstituted heteroaryl group, substituted or unsubstituted heterocyclylcarbonylethenyl, or polycyclic aryl group or is fused to an aryl ring and is optionally substituted by one or more additional groups.

In additional embodiments, the agent increases SIRT1 expression in the subject, for example in the cardiac muscle of the subject. In some such embodiments, the agent includes a nucleic acid molecule encoding a SIRT1 protein. For example, the agent can include a nucleic acid molecule encoding a SIRT1 protein including an amino acid sequence at least 90% identical to the amino acid sequence set forth as SEQ ID NO: 4, SEQ ID NO: 6, or SEQ ID NO: 8, wherein the SIRT1 protein deacetylates Nav1.5. In some embodiments the nucleic acid molecule is included on a vector, and the method includes administration of the vector to the subject with Brugada syndrome. Administration of the vector to the subject increases SIRT1 expression in the subject. In some examples, SIRT1 expression is increased in the cardiac muscle of the subject.

The foregoing and other objects, features, and advantages of the embodiments disclosed herein will become more apparent from the following detailed description, which proceeds with reference to the accompanying figures.

### BRIEF DESCRIPTION OF THE DRAWINGS

FIG. 1 is a schematic representation of Nav1.5, with localization of the mutations and associated phenotypes. Abbreviations: LQT, long QT syndrome; BrS, Brugada syndrome; CCD, cardiac conduction disease; DCM, dilated cardiomyopathy; MIX, mixed phenotype; SSD, sick sinus node syndrome. K, intracellular lysine residue mutated in some forms of Brugada syndrome (Adapted from Ruan et al., Nature Reviews Cardiology, 6, 337, 2009).

FIG. 2 is a schematic diagram illustrating the multiple targets and actions of SIRT1.

FIGS. 3A and 3B are a set of graphs showing whole cell patch clamp analysis showing that wild-type SIRT1 increases I_{Na} (FIG. 3A) and dominant negative SIRT1 decreases I_{Na} (FIG. 3B) in HEK 293 cells expressing Nav1.5. (n=7-10 cells each).

FIGS. 4A and 4B are a set of graphs showing whole cell patch clamp analysis showing that adenoviral overexpression of wild-type SIRT1 increases native I_{Na} (FIG. 4A), and that the SIRT1 inhibitor ex-243 (5 μM) decreases I_{Na} (FIG. 4B) in rat neonatal cardiac myocytes (n=5-7 cells each).

FIGS. 5A and 5B are a graph and a Western blot image showing that adenoviral expression of myc-SIRT1 in rat neonatal cardiomyocytes does not alter Nav1.5 expression at the (A) protein or (B) mRNA levels.

FIGS. 6A-6C are a graph and a set of digital images depicting results from (A) Immuno-luminescence and (B) immuno-fluorescence assays showing that wild-type SIRT1 increases, and dominant negative SIRT1 decreases, membrane localization of Nav1.5 in HEK 293 cells expressing extracellular FLAG-tagged Nav1.5. (C) Cell fractionation assay showing that SIRT1 increases native Nav1.5 in membrane fraction of rat neonatal cardiomyocytes. Insets: 60× magnification.

FIGS. 7A and 7B are a series of Western blot images showing that SIRT1 co-precipitates with Nav1.5. (7A) Full-length Nav1.5 was expressed in HEK 293 cells, with and without myc-tagged SIRT1. SIRT1 was immunoprecipitated (IP) with myc antibody. IPs were immunoblotted (IB) with Nav1.5 and myc. WCL: whole cell lysate. (B) Protein extracts from rat neonatal cardiac myocytes (top) or whole mouse heart (bottom) were immunoprecipitated with either non-specific IgG (N-IgG) or SCNA5 antibodies.

FIGS. 8A and 8B show a series of Western blot images illustrating that wild-type SIRT1 deacetylates full-length Nav1.5. (8A) GFP-tagged full-length Nav1.5 was expressed in HEK 293 cells, with and without myc-tagged wild-type SIRT1, and immunoprecipitated (IP). IPs were immunoblotted (IB) with GFP and myc. (8B) Nav1.5 was expressed in HEK 293 cells. Cells were co-transfected with SIRT or treated with resveratrol or NAM. Acetyl-lysine IPs were immunoblotted for Nav1.5

FIGS. 9A-9C are a graph and a set of Western blots showing that inhibition of endogenous SIRT1 increases lysine acetylation of native Nav1.5 in rat neonatal cardiomyocytes. (A) Ex-243 selectively inhibits SIRT1 activity in vitro. (B) Application of Ex-243 or (C) infection with a dominant negative SIRT1 increases lysine acetylation.

FIG. 10 is a set of Western blots showing that SIRT1 decreases lysine acetylation of loop III-IV of Nav1.5. GST-tagged Nav1.5 (III-IV) was expressed in HEK 293 cells. Cells were co-transfected with wild-type SIRT1 or treated with NAM or resveratrol. Nav1.5 (III-IV) was pulled down with GST-agarose, and immunoblotted (IB) with GST and acety-lysine antibodies.

FIG. 11 is a set of Western blots showing that SIRT1 targets loop III-IV of Nav1.5 for deacetylation in vitro. Purified GST-tagged Nav1.5 (III-IV) was acetylated in vitro by recombinant p300 acetyltransferase and acetyl-coA as the acetyl donor, followed by incubation with active recombinant SIRT1 and NAD⁺.

FIGS. 12A and 12B are a set of graphs showing that lysine 1479 of SIRT1 is acetylated and targeted by SIRT1. (A) detailed MS/MS spectrum revealing sequence and modification site of an acetylated peptide corresponding to 14 amino acids of Nav1.5 (¹⁴⁷⁹KLGGQDIFMTEEQK₁₄₉₂; SEQ ID NO: 9) (peak numbered 549.52, see arrow). Numbers signify mass/charge of detected peptides. Ac: acetyl group. (B) Decrease in quantity of the ionized peptide with SIRT1, suggesting that this acetylated peptide is a target of SIRT1.

FIGS. 13A and 13B are a set of Western blots showing that SIRT1 de-ubiquitinates Nav1.5. (A) HEK 293 cells were transfected with full-length GFP-tagged Nav1.5 and HA-ubiquitin. Cells were treated with resveratrol (100 μM, 4 hrs). Nav1.5 was immunoprecipitated (IP) with GFP and immunoblotted with GFP and HA. (B) HEK 293 cells were transfected with GST-tagged Nav1.5 (III-IV) and HA-ubiquitin. Cells were treated with NAM or co-transfected with SIRT1. Nav1.5 (III-IV) was pulled down with GST-agarose and immunoblotted (IB) with HA.

FIGS. 14A-14D are a series of Western blots and graphs showing results from cardiac specific SIRT1 knockout mice. (A) SIRT1 protein is decreased in the heart but not the kidney. (B) Acetylation of Nav1.5 protein immunoprecipitated from the heart is increased in cSIRT1^{-/-} mice. (C) PR interval is prolonged in anesthetized 3-month old mice. Tracings are signal averages of 10 beats. (D) High degree heart block in a cSIRT1^{-/-} mouse.

FIG. 15 is a graph showing that knockdown of GPD1-L in HEK 293 cells with siRNA increased I_{Na} compared to a scrambled construct (neg siRNA).

FIG. 16 shows a series of Western blots showing that SIRT1 and GPD1-L co-precipitate. Myc-SIRT1 and GPD1-L were co-expressed in HEK 293 cells. SIRT1 was immunoprecipitated (IP) with myc and immunoblotted (IB) with myc and GPD1-L. WCL: whole cell lysate.

FIG. 17 is a graph and a set of Western blots showing that GPD1-L (A280V) inhibits SIRT1 deacetylase activity. SIRT1 was expressed in HEK 293 cells, with and without WT and A280V GPD1L. Deacetylase activity was measured in SIRT1 immunoprecipitates with a fluorometric assay using acetylated p53 peptide as a substrate. Immunoprecipitated SIRT1 and expression of WT and A280V GPD1-L is shown at bottom.*P<0.05 (n=4).

FIG. 18 is a set of graphs showing expression of mRNA levels by real time PCR from human fibroblasts (fibro), undifferentiated iPS cells (iPS), and differentiated embryoid bodies with contracting regions (EB), normalized to whole human heart. The data represents the average of two independent experiments; each RT-PCR experiment was performed in duplicate.

FIGS. 19A and 19B are a set of graphs showing (A) phase contrast and immunofluorescence of a field of cells infected with AAV-cTn-GFP and plated at low density. Note selective fluorescence of iPS-CMs. (B) Whole cell Inward Na* current from whole cell voltage clamp of wild type IPS-derived cardiac myocytes. Cells were trypsinized and plated at low density Bottom: 20 ms depolarizing pulses were used from a holding potential of −80 mV to +40 mV in 5 mV increments for a representative cell. Top: I/V cure for a group of cells (n=5).

FIG. 20 is a schematic diagram illustrating shows Syndromes and disorders in which there is dysregulation of the cardiac sodium current.

FIG. 21 is a set of Western blots showing that SIRT1 targets lysine 1479 in Nav1.5 for deacetylation. HEK-293 cells transfected with GST-Naᵥ1.5-(III/IV) or GST-Naᵥ1.5-(III/IV)-K1479A and treated with SIRT1 siRNA to knock down SIRT1 or control siRNA. GST-tagged peptides were immunoprecipitated (IP) with GST-agarose and immunoblotted (IB) with GST or acetyl-lysine antibodies. Knockdown of SIRT1 was measured by immunoblotting for SIRT1 expression.

FIG. 22 is a graph showing that SIRT1 does not stimulate sodium current through Nav1.5 which is non-acetylatable on lysine 1479. Current Voltage (UV) relationship for peak I_{Na} taken from HEK-293 cells transfected with wt-Naᵥ1.5 (n=8), Naᵥ1.5+SIRT1 (n=9), K1479A-Naᵥ1.5 (n=9) or K1479A-Naᵥ1.5+SIRT1 (n=11).

FIG. 23 is a graph showing that dominant negative SIRT1 does not decrease sodium current through Nav1.5 which is non-acetylatable on lysine 1479. I/V relationship for peak I_{Na} from whole cell patch clamp recordings of HEK-293 cells expressing: Naᵥ1.5 (n=8), Naᵥ1.5+H363Y-SIRT1 (N=9), K1479A-Naᵥ1.5 (N=11), K1479A-Naᵥ1.5+H363Y-SIRT1 (n=10).

FIG. 24 is a graph showing decreased conduction velocity in hearts of mice deficient for SIRT1. Conduction velocity of the cardiac action potential was measured by optical mapping at standard pacing protocol (200 ms) in hearts of wild type (WT) mice and mice with cardiomyocyte-specific knockout of SIRT1 (cSIRT1^{-/-}).

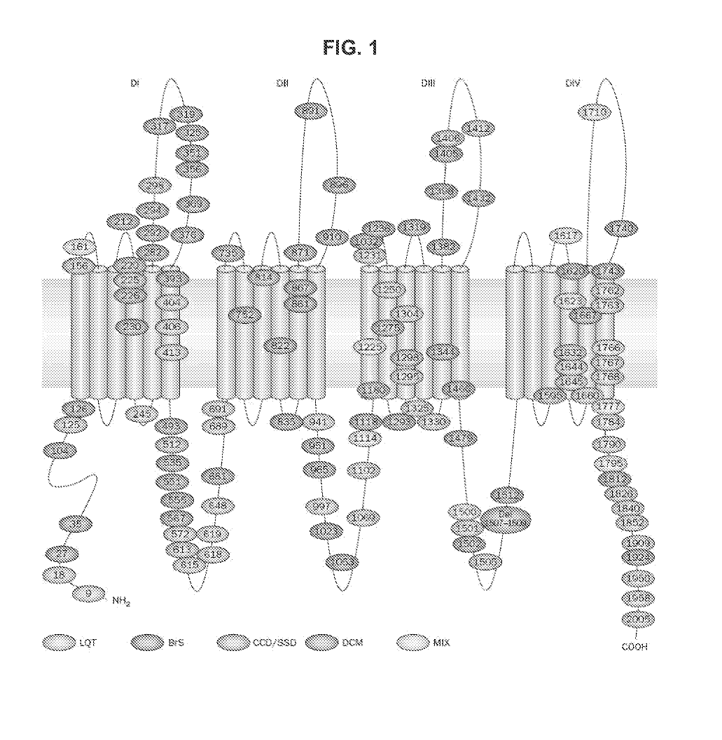

### SEQUENCE LISTING

The nucleic acid and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, Annex C/St.25 text file, created on May 6, 2013, ˜146 KB, which is incorporated by reference herein. In the accompanying sequence listing:

SEQ ID NO: 1 is the amino acid sequence of C. elegans Sir2 (GENBANK™ Accession No. P53685, incorporated by reference herein as present in GENBANK™ on May 6, 2012)

SEQ ID NO: 2 is the amino acid sequence of C. elegans Sir2.1 (GENBANK™ Accession No. NP_{—}501912 incorporated by reference herein as present in GENBANK™ on May 6, 2012).

SEQ ID NO: 3 is an exemplary cDNA sequence encoding human SIRT1 (GENBANK™ Accession No. NM_{—}012238 incorporated by reference herein as present in GENBANK™ on May 6, 2012

SEQ ID NO: 4 is the amino acid sequence of human SIRT1 (GENBANK™ Accession No. NP_{—}036370, incorporated by reference herein as present in GENBANK™ on May 6, 2012).

SEQ ID NO: 5 is an exemplary cDNA sequence encoding human SIRT2, variant 1 (GENBANK™ Acc. No. NM_{—}012237, incorporated by reference herein as present in GENBANK™ on May 6, 2012).

SEQ ID NO: 6 is the amino acid sequence of human SIRT2, variant 1 (GENBANK™ Acc. No. NP_{—}036369 incorporated by reference herein as present in GENBANK™ on May 6, 2012).

SEQ ID NO: 7 is an exemplary cDNA sequence encoding human SIRT2, variant 2 (GENBANK™ Acc. No. NM_{—}030593 incorporated by reference herein as present in GENBANK™ on May 6, 2012).

SEQ ID NO: 8 is the amino acid sequence of human SIR2, variant 2 (GENBANK™ Acc. No. NP_{—}085096, incorporated by reference herein as present in GENBANK™ on May 6, 2012).

SEQ ID NO: 9 is the amino acid sequence of a peptide.

SEQ ID NO: 10 is the amino acid sequence the sodium channel protein type 5 subunit alpha isoform a.

SEQ ID NO: 11 is an exemplary nucleic acid sequence encoding the sodium channel protein type 5 subunit alpha isoform a.

SEQ ID NO: 12 is the amino acid sequence the sodium channel protein type 5 subunit alpha isoform b.

SEQ ID NO: 13 is the amino acid sequence the sodium channel protein type 5 subunit alpha isoform c.

SEQ ID NO: 14 is the amino acid sequence the sodium channel protein type 5 subunit alpha isoform d.

SEQ ID NO: 15 is the amino acid sequence the sodium channel protein type 5 subunit alpha isoform e.

SEQ ID NO: 16 is the amino acid sequence the sodium channel protein type 5 subunit alpha isoform f.

### DETAILED DESCRIPTION

The cardiac Na⁺ channel Nav1.5 (Nav1.5) and the inward depolarizing Na⁺ current (I_{Na}) play a critical role in regulating the action potential of myocytes in the atrium, ventricle, and in maintaining rapid conduction velocity throughout the heart (Amin et al., Pflugers Arch, 460, 223-237, 2010). Gain of function mutations in Nav1.5 that increase late currents are associated with type 3 long QT syndrome. Loss of function mutations in Nav1.5 that decrease I_{Na} are associated with cardiac arrhythmias, and cause ˜20% of cases of Brugada syndrome, a smaller fraction of cases of isolated conduction system disease, and rarely dilated cardiomyopathy. These mutations can affect protein expression, channel function, and/or channel trafficking to the membrane.

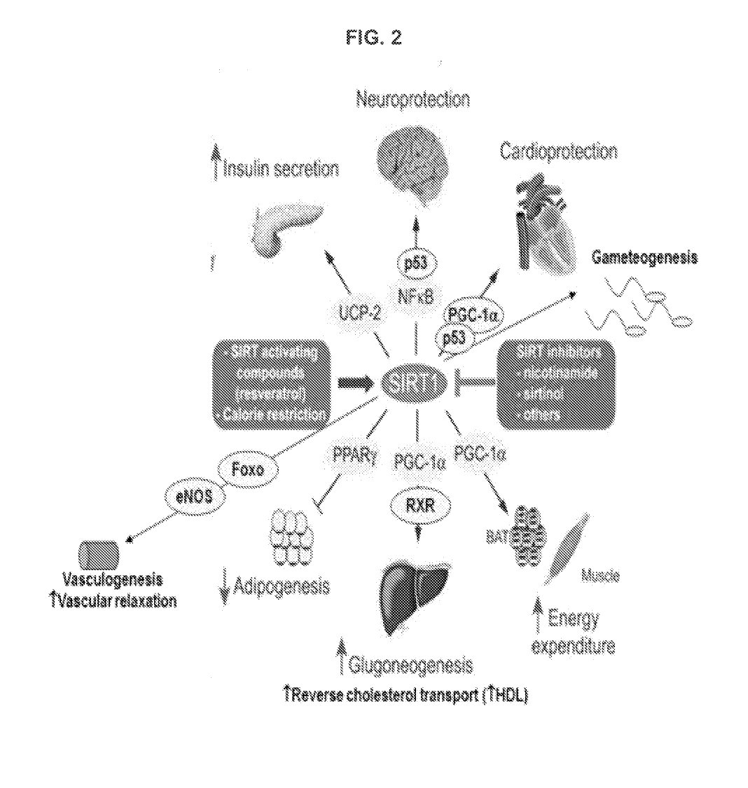

Silencing Information Regulators (SIR) are a family of histone deacetylases (HDACs), first identified in yeast, that are collectively known as SIRTUIN proteins. Unlike HDACs of other classes, SIRTUINs require nicotinamide adenine dinucleotide (NAD⁺) as a co-factor, and form a family of class III nicotinamide adenine dinucleotide (NAD+)-dependent protein deacetylases. Nicotinamide (NAM), one of the products of this reaction, feeds back to inhibit the activity of these deacetylases (FIG. 2).

SIRT1 (SIRTUIN1) is the closest mammalian homologue of yeast Sir2, and is a ubiquitously expressed mammalian deacetylase that targets specific acetylated lysine residues on histones and non-histone proteins. SIRT1 is regulated by energy (NAD) availability and is pivotal in energy homeostasis. In addition to deacetylating histones, SIRT1 targets many non-histone proteins such as p53 (Vaziri et al., Cell, 107, 149-159, 2001, forkhead transcription factors (FoxOs; Motta et al., Cell, 116, 551-563, 2004), Bax (Cohen et al., Science, 305, 390-392, 2004), PGC-1α (Rodgers et al., Nature, 434, 113-118, 2005), and PPAR (Picard et al., Nature, 429, 771-776, 2004), among others (see FIG. 2). SIRT1 impacts the cardiovascular system both directly and indirectly, the latter by modulating whole body metabolism through regulation of the activities of these transcription factors, co-regulators, and enzymes that improve energy homeostasis in adipose tissue, liver, skeletal muscle, and pancreas. SIRT1 controls myocardial development and protects against stress- and aging-associated myocardial dysfunction (Hariharan et al., Circ Res, 107, 1470-1482, 2010) through the deacetylation of p53 and FoxO factors (Borradaile et al., Curr Pharm Des, 15, 110-117, 2009). Moreover, by modulating the activity of endothelial nitric oxide synthase (eNOS) (Mattagajasingh et al., Proc Natl Acad Sci USA, 104, 14855-14860, 2007, FoxO transcription factors (Potente, Genes Dev, 21, 2644-2658, 2007), SIRT1 also promotes vasodilatory and regenerative functions in endothelial and smooth muscle cells of the vascular wall. Despite rapidly expanding knowledge about SIRT1's role in the cardiovascular biology, little is known about its function in cardiac excitability.

It is disclosed herein that SIRT1 can deacetylate Nav1.5, that Nav1.5 and SIRT1 co-immunoprecipitate, that over-expression of SIRT1 increases I_{Na} currents and that dominant negative suppression of SIRT1 or use of SIRT1 inhibitors decreases I_{Na} currents, that SIRT1 increases membrane expression of Nav1.5 and dominant negative SIRT1 decreases membrane expression of Nav1.5, that SIRT1 targets lysine 1479 in Nav1.5, that SIRT1 increases I_{Na} and dominant negative SIRT1 decreases I_{Na} by targeting lysine 1479 in Nav1.5, and that SIRT1 increases membrane expression of Nav1.5 by targeting lysine 1479 in Nav1.5. In addition there is prolongation of the PR interval, intermittent high-grade atrioventricular block on ECGs and telemetry of cardiomyocyte-specific SIRT1 knockout mice compared to wild type control mice, and decreased conduction velocity of the electrical impulse in hearts of cardiomyocyte-specific SIRT1 knockout mice compared to wild type control mice. Finally, the SIRT1 activator resveratrol leads to increased QRS amplitude on ECGs suggestive of increased sodium currents.

Accordingly, it is disclosed herein that agents that increase the expression and or activity of SIRT1, including SIRT1 itself, can be used as therapies for arrhythmia syndromes, particularly arrhythmia syndromes associated with decreased expression or activity of Nav1.5 protein, such as Brugada syndrome. For inherited syndromes such as Brugada syndrome where decreased sodium current is the direct cause of cardiac arrhythmia, agents that increase the activity and/or expression of SRIT1 can be particularly beneficial.

### I. TERMS

Unless otherwise noted, technical terms are used according to conventional usage. Definitions of common terms in molecular biology may be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710). The term “comprises” means “includes.” Unless context indicates otherwise, and to facilitate review of the various embodiments of this disclosure, the following explanations of terms are provided:

Administration: The introduction of a composition or agent into a subject by a chosen route. Administration can be local or systemic. For example, if the chosen route is intravenous, the composition is administered by introducing the composition into a vein of the subject. In some examples a disclosed agent that increases SIRT1 expression or activity is administered to a subject.

Agent: Any substance or any combination of substances that is useful for achieving an end or result; for example, a substance or combination of substances useful for activating SIRT1 activity or expression in a subject. Agents include proteins, nucleic acid molecules, compounds, small molecules, organic compounds, inorganic compounds, or other molecules of interest. An agent can include a therapeutic agent (such as an agent that increases SIRT1). The skilled artisan will understand that particular agents may be useful to achieve more than one result.

Agent that increases SIRTUIN: A molecule, such as a compound, that increases the level of a SIRTUIN protein and/or increases the deacetylase activity of a SIRTUIN protein, such as SIRT1. In an exemplary embodiment, an agent that increases SIRTUIN can increase the deacetylase activity of a SIRTUIN protein by at least about 10%, 25%, 50%, 75%, 100%, or more. Agents that increase the deacetylase activity a SIRTUIN protein are SIRTUIN activators, such as SIRT1 activators as disclosed herein. Exemplary biological activities of SIRTUIN proteins include deacetylation, e.g., of histones and p53.

Agent that inhibits SIRTUIN: A molecule, such as a compound, that decreases the level of a SIRTUIN protein and/or decreases at least one activity of a SIRTUIN protein. In an exemplary embodiment, the agent can decrease at least one biological activity of a SIRTUIN protein by at least about 10%, 25%, 50%, 75%, 100%, or more. Exemplary biological activities of SIRTUIN proteins include deacetylation, e.g., of histones and p53; extending lifespan; increasing genomic stability; silencing transcription; and controlling the segregation of oxidized proteins between mother and daughter cells.

Amino acid substitution: The replacement of one amino acid in polypeptide with a different amino acid.

Animal: Living multi-cellular vertebrate organisms, a category that includes, for example, mammals and birds. The term mammal includes both human and non-human mammals. Similarly, the term “subject” includes both human and veterinary subjects.

Arrhythmia syndrome: A condition of abnormal electrical activity in the heart. The abnormal electrical activity leads to an irregular heartbeat or uncoordinated myocardial contraction, as in a ventricular defibrillation. In some conditions, the abnormal electrical activity in the heart includes a decrease in I_{Na} compared to a control (such as the I_{Na} a healthy subject). In some embodiments, the arrhythmia syndrome includes tachyarrhythmia (rapid heartbeat, e.g., more than 100 beats per minute, with arrhythmia) or bradyarrhythmia (slow heartbeat, e.g., less than 60 beats per minute, with arrhythmia). One non-limiting example of an arrhythmia syndrome is Brugada syndrome. In another example, an arrhythmia syndrome is an arrhythmia syndrome involving decreased inward depolarizing Na⁺ current (I_{Na}) through cardiac sodium channels, such as the Nav1.5 channel.

Arrhythmia syndrome due to sodium channel deficiency: An arrhythmia syndrome resulting from a reduced cardiac sodium channel activity. For example, the reduction in activity can be due to downregulation of the sodium channel or decreased activity of the sodium channel. For example, the downregulation or decreased activity of the sodium channel can be due to a mutation in the gene encoding the sodium channel. In one example, the sodium channel includes the Nav1.5 channel. The arrhythmia syndrome due to sodium channel deficiency can be an inherited or acquired arrhythmia syndrome. Arrhythmia syndrome due to sodium channel deficiency, and methods of identifying a subject with such syndromes are familiar to the person of ordinary skill in the art (see, e.g., Bezzina et al., Cardiovascular Res., 49:257-271, 2001; Remme et al., Cardiovascular Therapeutics, 28:287-294, 2010; Deovendans and Wilde (Eds), Cardiovascular Genetics for Clinicians, Springer, 2012; Baars, Deovendans, and Smaagt (Eds), Clinical Cardiogenetics, Springer, 2011; and Bruker and Tavora (Eds), Practical Cardiovascular Pathology, Lippincott Williams & Wilkins, 2010, each of which is incorporated by reference herein). Non-limiting examples of arrhythmia syndromes due to sodium channel deficiency include tachyarrhythmia in inherited Brugada syndrome, bradyarrhythmia in inherited conduction disease, tachyarrhythmia and bradyarrhythmia in inherited heart failure due to sodium channel mutations, tachyarrhythmia and bradyarrhythmia in acquired nonischemic cardiomyopathies with sodium channel downregulation, and tachyarrhythmia and bradyarrhythmia in ischemic cardiomyopathy patients with sodium channel downregulation.

Brugada Syndrome: A genetic disease that is characterized by abnormal electrocardiogram (ECG) findings and an increased risk of sudden cardiac death, often from ventricular fibrillation. Approximately 20% of the cases of Brugada syndrome have been shown to be associated with mutation(s) in the SCN5A gene that encodes for the sodium ion channel in the cell membranes of the muscle cells of myocytes. Loss-of-function mutations in this gene lead to a loss of the action potential dome of some epicardial areas of the right ventricle. Brugada syndrome has 3 different ECG patterns: Type 1 has a coved type ST elevation with at least 2 mm (0.2 mV) J-point elevation a gradually descending ST segment followed by a negative T-wave; Type 2 has a saddle back pattern with a least 2 mm J-point elevation and at least 1 mm ST elevation with a positive or biphasic T-wave. Type 2 pattern can occasionally be seen in healthy subjects; and Type 3 has either a coved (type 1 like) or a saddle back (type 2 like) pattern with less than 2 mm J-point elevation and less than 1 mm ST elevation. Type 3 pattern is not uncommon in healthy subjects. Methods of identifying a subject with Brugada syndrome are known to the person of ordinary skill in the art; see, e.g., Antzelevitch et al. (Eds.), The Brugada Syndrome: From Bench to Bedside, Wiley-Blackwell, 2005.

Conservative amino acid substitutions: “Conservative” amino acid substitutions are those substitutions that do not substantially affect or decrease the function of a protein. For example, the enzymatic activity of a protein, such as deacetylase activity. For example, a variant polypeptide that includes deacetylase enzymatic activity can include up to one, up to two, up to three, up to four, or up to five conservative amino acid substitutions, or at most about 1, at most about 2, at most about 3 at most about 4, at most about 5, at most about 10, or at most about 15 conservative substitutions and retain deacetylase activity.

Furthermore, one of ordinary skill will recognize that individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

Conservative amino acid substitution tables providing functionally similar amino acids are well known to one of ordinary skill in the art. The following six groups are examples of amino acids that are considered to be conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

Non-conservative substitutions are those that reduce an activity or function of a protein, such as enzymatic activity of the protein. For instance, if an amino acid residue is essential for a function of the protein, even an otherwise conservative substitution may disrupt that activity. Thus, a conservative substitution does not alter the basic function of a protein of interest.

Control: A reference standard. In some embodiments, the control is a sample obtained from a healthy patient. In other embodiments, the control is a tissue sample obtained from a patient with a disease, such as a patient diagnosed with an arrhythmia syndrome, such as Brugada syndrome. In still other embodiments, the control is a historical control or standard reference value or range of values (such as a previously tested control sample, such as a group of patients with an arrhythmia syndrome, such as Brugada syndrome with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Expression: Translation of a nucleic acid into a protein. Proteins can be expressed and remain intracellular, can become a component of the cell surface membrane, or be can secreted into the extracellular matrix or medium.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signal for introns, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Inhibiting or treating a disease: Inhibiting a disease, such as Brugada syndrome, refers to inhibiting the full development of a disease. In several examples, inhibiting a disease refers to lessening an arrhythmia syndrome, such as preventing the development, progression, or severity of an arrhythmia syndrome, such as Brugada syndrome, in a person who is known to have an arrhythmia syndrome, such as Brugada syndrome, or who has a gene mutation associated with an arrhythmia syndrome, such as Brugada syndrome, or lessening a sign or symptom of the disease. “Treatment” refers to a therapeutic intervention that ameliorates a sign or symptom of a disease or pathological condition related to the disease, such as an arrhythmia syndrome, such as Brugada syndrome. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, such as a reduction in cardiac arrhythmia, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

Isolated: An “isolated” biological component (such as a nucleic acid or protein or organelle) has been substantially separated or purified away from other biological components in the cell of the organism in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Nav1.5: A sodium ion channel protein that in humans is encoded by the SCN5A gene. Mutations in the gene are associated with long QT syndrome type 3 (LQT3), Brugada syndrome, primary cardiac conduction disease and idiopathic ventricular fibrillation. The Nav1.5 protein encoded by the SCN5A gene is an integral membrane protein and tetrodotoxin-resistant voltage-gated sodium channel subunit. The encoded protein is found primarily in cardiac muscle and is responsible for the initial upstroke of the action potential in an electrocardiogram. Defects in this gene are known to cause arrhythmia syndromes, including Brugada syndrome. The person of ordinary skill in the art is familiar with Nav1.5 protein and the encoding SCN5A gene, and their functions see, e.g., Rook et al., Cardiovascular Res., 93:12-23, 2012). The sequence of the SCN5A gene is known, see, e.g., GENBANK™ Gene ID NO. 6331, incorporated by reference herein as present in GENBANK on May 5, 2013.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together. This includes nucleic acid vectors comprising an amplified or assembled nucleic acid which can be used to transform a suitable host cell. A host cell that comprises the recombinant nucleic acid is referred to as a “recombinant host cell.” The gene is then expressed in the recombinant host cell to produce, e.g., a “recombinant polypeptide.” A recombinant nucleic acid may serve a non-coding function (e.g., promoter, origin of replication, ribosome-binding site, etc.) as well.

A first sequence is an “antisense” with respect to a second sequence if a polynucleotide whose sequence is the first sequence specifically hybridizes with a polynucleotide whose sequence is the second sequence.

Terms used to describe sequence relationships between two or more nucleotide sequences or amino acid sequences include “reference sequence,” “selected from,” “comparison window,” “identical,” “percentage of sequence identity,” “substantially identical,” “complementary,” and “substantially complementary.”

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds 1995 supplement)).

One example of a useful algorithm is PILEUP. PILEUP uses a simplification of the progressive alignment method of Feng & Doolittle, J. Mol. Evol. 35:351-360, 1987. The method used is similar to the method described by Higgins & Sharp, CABIOS 5:151-153, 1989. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395, 1984.

Another example of algorithms that are suitable for determining percent sequence identity and sequence similarity are the BLAST and the BLAST 2.0 algorithm, which are described in Altschul et al., J. Mol. Biol. 215:403-410, 1990 and Altschul et al., Nucleic Acids Res. 25:3389-3402, 1977. Software for performing BLAST analyses is publicly available through the National Center for Biotechnology Information (ncbi.nlm.nih.gov). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915, 1989). An oligonucleotide is a linear polynucleotide sequence of up to about 100 nucleotide bases in length.

A polynucleotide or nucleic acid sequence refers to a polymeric form of nucleotide at least 10 bases in length. A recombinant polynucleotide includes a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides can be ribonucleotides, deoxyribonucleotides, or modified forms of either nucleotide. The term includes single- and double-stranded forms of DNA.

Operably linked: A first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington's Pharmaceutical Sciences, by E. W. Martin, Mack Publishing Co., Easton, Pa., 15th Edition (1975), describes compositions and formulations suitable for pharmaceutical delivery of the agents disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

A “therapeutically effective amount” is a quantity of a composition to achieve a desired effect in a subject being treated. For instance, this can be the amount of SIRT1 activator necessary to treat or reduce cardiac arrhythmia associated with an arrhythmia syndrome, such as Brugada syndrome. When administered to a subject, a dosage will generally be used that will achieve target tissue concentrations (for example, in cardiac tissue) that has been shown to achieve an in vitro effect.

Polynucleotide: The term polynucleotide or nucleic acid sequence refers to a polymeric form of nucleotide at least 10 bases in length. A recombinant polynucleotide includes a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides can be ribonucleotides, deoxyribonucleotides, or modified forms of either nucleotide. The term includes single- and double-stranded forms of DNA.

Polypeptide: A chain of amino acids, generally eight to 20 amino acids in length, which can be post-translationally modified (e.g., glycosylation or phosphorylation).

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of a polypeptide (such as an therapeutic polypeptide) that specifically binds another polypeptide (such as the KDEL receptor) are typically characterized by possession of at least about 75%, for example at least about 80%, 85%, 90%, 91%, 92%, 93%, 94%, 95%, 96%, 97%, 98% or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

SIRTUIN deacetylase: A family of class III nicotinamide adenine dinucleotide (NAD+)-dependent protein deacetylases. Unlike HDACs of other classes, SIRTUINs require nicotinamide adenine dinucleotide (NAD⁺) as a co-factor. The prototypical member of this family is the yeast SIR2 protein (GENBANK™ Accession No. P53685, incorporated by reference herein as present in GENBANK on May 6, 2012). Other members include C. elegans Sir-2.1 (GENBANK™ Accession No. NP_{—}501912, incorporated by reference herein as present in GENBANK on May 6, 2012), human SIRT1 (GENBANK™ Accession No. NM_{—}012238 and NP_{—}036370 (or AF083106, incorporated by reference herein)) and SIRT2 (GENBANK™ Accession No. NM_{—}012237, NM_{—}030593, NP_{—}036369, NP_{—}085096, and AF083107, incorporated by reference herein as present in GENBANK on May 6, 2012) proteins. Other family members include the four additional yeast Sir2-like genes termed “HST genes” (homologues of Sir two) HST1, HST2, HST3 and HST4, and the five other human homologues hSIRT3, hSIRT4, hSIRT5, hSIRT6 and hSIRT7 (see, e.g., Brachmann et al. Genes Dev. 9:2888, 1995 and Frye et al. BBRC 260:273, 1999).

SIRT1: A ubiquitously expressed mammalian deacetylase that targets specific acetylated lysine residues on histones and non-histone proteins. SIRT1 is also known as SIRTUIN1, and is the closest mammalian homologue of yeast Sir2. SIRT1 is the largest of the members of the SIRTUIN family of class III nicotinamide adenine dinucleotide (NAD+)-dependent protein deacetylases. Non-limiting examples of SIRT1 protein include human SIRT1 (GENBANK™ Accession No. NM_{—}012238, SEQ ID NO: 3 and NP_{—}036370, SEQ ID NO: 4), human SIRT2, variant 1 (GENBANK™ Accession No. NM_{—}012237, SEQ ID NO: 5 (cDNA); and NP_{—}036369 (protein); SEQ ID NO: 6), and human SIRT2, variant 2 (GENBANK™ Accession No. NM_{—}030593 (cDNA), SEQ ID NO: 7; and NP_{—}085096 (protein), SEQ ID NO: 8) proteins, and equivalents and fragments thereof. SIRT1 protein is familiar to the person of ordinary skill in the art, see, e.g., Haigis and Sinclair, Ann. Rev. Pathol., 5:253-295, 2010; and Michan and Sinclair, Biochem J., 15:1-13, 2007).

Subject: Living multi-cellular vertebrate organisms, a category that includes human and non-human mammals. In one example, a subject is a human. In an additional example, a subject is selected that is in need of inhibition of a neurodegenerative disorder or myocardial infarction. For example, the subject is either at risk of or has neurodegenerative disorder, or myocardial infarction.

Therapeutically Effective Amount: An amount of a composition that alone, or together with an additional therapeutic agent(s) induces the desired response (e.g., inhibition of cardiac arrhythmia). In several embodiments, a therapeutically effective amount is the amount necessary to inhibit a sign or symptom of an arrhythmia syndrome, such as Brugada syndrome, for example, to inhibit cardiac arrhythmia in a subject. When administered to a subject, a dosage will generally be used that will achieve target tissue concentrations that has been shown to achieve a desired in vitro effect. In one example, a desired response is to inhibit cardiac arrhythmia associated with an arrhythmia syndrome, such as Brugada syndrome. The cardiac arrhythmia does not need to be completely inhibited for the composition to be effective. For example, a composition can decrease cardiac arrhythmia associated with an arrhythmia syndrome, such as Brugada syndrome by a desired amount, for example by at least 10%, at least 20%, at least 50%, at least 60%, at least 70%, at least 80%, at least 90%, at least 95%, at least 98%, or even at least 100% (elimination of Brugada syndrome or cardiac arrhythmia), as compared to a control, such as the cardiac arrhythmia in the subject before treatment, or in a subject with an arrhythmia syndrome, such as Brugada syndrome, in the absence of the composition. A therapeutically effective amount of an agent can be administered in a single dose, or in several doses, for example daily, during a course of treatment. However, the therapeutically effective amount can depend on the subject being treated, the severity and type of the condition being treated, and the manner of administration.

Under conditions sufficient for: A phrase used to describe any environment or set of conditions that permits the desired activity or outcome.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art.

### II. THERAPEUTIC METHODS

Methods are provided herein for treating an arrhythmia syndrome in a subject, for example a ventricular arrhythmia syndrome, such as for treating Brugada syndrome in a subject. In several embodiments, the methods include administering to the subject a therapeutically effective amount of an agent that increases the expression or activity of a SIRTUIN protein (such as SIRT1) in the subject. In several embodiments, the agent is a SIRT1 activator. In additional embodiments, the agent is an expression vector encoding a SIRTUIN protein, such as SIRT1. In some embodiments, the agent increases sodium channel activation in cardiac muscle in the subject. In some embodiments, the agent increases the expression or activity of SIRT1 and increases sodium channel activation in cardiac muscle in the subject.

In further embodiments, the methods include administering to the subject a therapeutically effective amount of an agent including an expression vector encoding a mutated Nav1.5 protein that is non-acetylatable on lysine residue 1479. This form of Nav1.5, by being non-acetylatable on lysine residue 1479, is not subject to down-regulation by endogenous acetylases, and functions as a constitutive sodium channel, increasing I_{Na}.

Several of the disclosed methods of treating or inhibiting arrhythmia syndromes (such as a method of treating or inhibiting Brugada syndrome) include selecting a subject with cardiac arrhythmia for treatment. The person of ordinary skill in the art is familiar with methods of identifying a subject with cardiac arrhythmia, such as methods of identifying a subject with Brugada syndrome (see, e.g., Brugada (Ed.), Clinical Approach to Sudden Death Syndromes, Springer, 2010; Dubin (Ed), Rapid Interpretation of EKG's, 6^{th}, Cover Pub. Co., 2000; and Antzelevitch et al. (Eds.), The Brugada Syndrome: From Bench to Bedside, Wiley-Blackwell, 2005).

In several embodiments, the methods include treatment of inherited or acquired arrhythmia syndromes due to cardiac sodium channel deficiencies (such as an arrhythmia syndrome involving decreased sodium current through the Nav1.5 channel). In some embodiments, the arrhythmia syndrome involves tachyarrhythmia in inherited Brugada syndrome. In additional embodiments, the arrhythmia syndrome involves bradyarrhythmia in inherited conduction disease. In further embodiments, the arrhythmia syndrome involves tachyarrhythmia and/or bradyarrhythmia in inherited heart failure due to sodium channel mutations. In other embodiments, the arrhythmia syndrome involves tachyarrhythmia and/or bradyarrhythmia in acquired nonischemic cardiomyopathies with sodium channel downregulation. In some embodiments, the arrhythmia syndrome involves tachyarrhythmia and/or bradyarrhythmia in ischemic cardiomyopathy patients with sodium channel downregulation. For all of these subsets of arrhythmia syndromes, increasing sodium currents using sirtuin activators as disclosed herein provides a novel mechanism to prevent arrhythmias.

In some embodiments, the methods include selecting/and or treating a subject with Brugada syndrome. Mutations in 10 genes have been linked to Brugada syndrome (see Table 1). Mutations in SCN5A (see, e.g., Chen et al., Nature; 392: 293-296, 1998) leading to a loss of function of the cardiac sodium (Na⁺) channel by different mechanisms is the most common genotype found among these patients (ie, ≈20% of BS cases; range 11-28%). To date, almost 300 mutations in SCN5A have been described in association with BS (see, e.g., Kapplinger et al., Heart Rhythm, 7: 33-46, 2010). Mutations in the glycerol-3-phosphate dehydrogenase 1-like gene (GPD1L) cause abnormal trafficking of the cardiac Na+ channel to the cell surface and a reduction of approximately 50% of the inward Na+ current (see, e.g., London et al., Circulation, 116: 2260-2268, 2007). Mutations in genes encoding the α1- (CACNA1c) and β2b- (CACNB2b) subunits of the L-type cardiac calcium (Ca2+) channel leading to a decrease of the I_{ca} current, result in a combined BS/short QT syndrome (see, e.g., Antzelevitch et al., Circulation, 115: 442-449, 2007). Other genes recently reported to be linked to the syndrome are: SCN1B (encoding for β1- and β1b-subunits, auxiliary function-modifying subunits of the cardiac Na+ channel, resulting in a decrease of the I_{Na} current by affecting the Na+ channel trafficking (see, e.g., Watanabe et al., J Clin Invest, 118: 2260-2268, 2008); KCNE3 (see, e.g., Delpón et al., Circ Arrhythm Electrophysiol, 1: 209-218, 2008; encoding MiRP2, a protein that decreases the potassium (K+) transient outward current (Iₜₒ) current by interacting with channel Kv4.3, resulting in an increase of Iₜₒ magnitude and density (see, e.g., Delpón et al., Circ Arrhythm Electrophysiol, 1: 209-218, 2008); SCN3B (which encodes for the β3-subunit of the Na+ cardiac channel, and leading to a loss of function of the Na+ cardiac channel also cause Brugada syndrome (see, e.g., Hu et al., Circ Cardiovasc Genet., 2: 270-278, 2009); MOG1 (see, e.g., Kattygnarath et al., Circ Cardiovasc Genet., 4: 261-268, 2011; mutations in this gene cause INa reduction by impairing the trafficking of the cardiac Na+ channel to the cell membrane); KCNE5 (see, e.g., Ohno et al., Circ Arrhythm Electrophysiol., 4: 352-361, 2011); and KCND3 (see, e.g., Giudicessi et al., Heart Rhythm, 8: 1024-1032, 2011; mutations in both genes leading to an increase of the Iₜₒ current have been linked to BS).

TABLE 1
Genes linked to Brugada syndrome

| Variant | Gene | Ionic current |
|---|---|---|
| BS1 | SCN5A | I_{Na} |
| BS2 | GPD1-L | I_{Na} |
| BS3 | CACNA1c | I_{Ca} |
| BS4 | CACNB | I_{Ca} |
| BS5 | SCN1B | I_{Na} |
| BS6 | KCNE3 | Ito |
| BS7 | SCN3B | I_{Na} |
| BS8 | MOG1 | I_{Na} |
| BS9 | KCNE5 | Iₜₒ |
| BS10 | KCND3 | Iₜₒ |

In some embodiments, selecting a subject with Brugada syndrome includes selecting a subject with Brugada syndrome caused by a mutation in one or more of the SCN5A, GPD1-L, CACNA1c, CACNB, SCN1B, KCNE3, SCN3B, MOG1, KCNE5, or KCND3 genes. In some embodiments, selecting a subject with Brudaga syndrome includes selecting a subject with Brudaga syndrome that has reduced I_{Na} current in cardiac muscle. In some embodiments, selecting a subject with Brugada syndrome includes selecting a subject with Brugada syndrome caused by a mutation in one or more of the SCN5A, GPD1-L, SCN1B, SCN3B, or MOG1 genes. In some embodiments, selecting a subject with Brugada syndrome includes selecting a subject with Brugada syndrome caused by a mutation in the SCN5A gene, the GPD1-L gene, the SCN1B gene, the SCN3B gene or the MOG1 gene. The person of ordinary skill in the art can readily determine if a subject with Brugada syndrome has a mutation in any of the above mentioned genes using conventional methods familiar in the art. In some embodiments, selecting a subject with Brudaga syndrome includes selecting a subject with a particular type of Brudaga syndrome, such as type BS1, BS2, BS3, BS4, BS5, BS6, BS7, BS8, BS9, or BS10 Brugada syndrome (see, e.g., Berne and Brugada, Circ. J. 76, 1563-1571, 2012 for review).

The therapeutically effective amount of the agent that increases the expression or activity of a SIRTUIN protein (such as SIRT1) in the subject will depend upon the severity of the disease and the general state of the patient's health. A therapeutically effective amount of the agent that increases the expression or activity of a SIRTUIN protein (such as SIRT1) in the subject is that which provides either subjective relief of a symptom(s) or an objectively identifiable improvement as noted by the clinician or other qualified observer. In one embodiment, a therapeutically effective amount of an agent that increases the expression or activity of a SIRTUIN protein (such as SIRT1) in the subject is the amount necessary to inhibit cardiac arrhythmia in the subject. The therapeutically effective amount of the agents administered can vary depending upon the desired effects and the subject to be treated. In some examples, therapeutic amounts are amounts which eliminate or reduce the patient's arrhythmia, or which prevent or reduce the arrhythmia in the subject. The person of ordinary skill in the art will appreciate that the arrhythmia syndrome (e.g., Brugada syndrome) does not need to be completely eliminated for successful treatment or inhibition to occur.

The therapeutically effective amount of an agent including an expression vector encoding a mutated Nav1.5 protein that is non-acetylatable on lysine residue 1479 will depend upon the severity of the disease and the general state of the patient's health. A therapeutically effective amount of the agent including the expression vector encoding a mutated Nav1.5 protein that is non-acetylatable on lysine residue 1479 is that which provides either subjective relief of a symptom(s) or an objectively identifiable improvement as noted by the clinician or other qualified observer. In one embodiment, a therapeutically effective amount of the agent including the expression vector encoding a mutated Nav1.5 protein that is non-acetylatable on lysine residue 1479 is the amount necessary to inhibit cardiac arrhythmia in the subject. The therapeutically effective amount of the agent including the expression vector encoding a mutated Nav1.5 protein that is non-acetylatable on lysine residue 1479 administered can vary depending upon the desired effects and the subject to be treated. In some examples, therapeutic amounts are amounts which eliminate or reduce the patient's arrhythmia, or which prevent or reduce the arrhythmia in the subject. The person of ordinary skill in the art will appreciate that the arrhythmia syndrome (e.g., Brugada syndrome) does not need to be completely eliminated for successful treatment or inhibition to occur.

Subjects that can benefit from the disclosed methods include human and veterinary subjects. Subjects can be screened prior to initiating the disclosed therapies, for example to determine whether the subject has an arrhythmia syndrome, such as Brugada syndrome. The presence of the arrhythmia syndrome indicates that the syndrome can be treated using the methods provided herein.

Any method of administration can be used for the disclosed conjugates, antibodies, compositions and additional agents, including local and systemic administration. For example topical, oral, intravascular such as intravenous, intramuscular, intracardiac, intraperitoneal, intranasal, intradermal, intrathecal and subcutaneous administration can be used. The particular mode of administration and the dosage regimen will be selected by the attending clinician, taking into account the particulars of the case (for example the subject, the disease, the disease state involved, and whether the treatment is prophylactic). In cases in which more than one agent or composition is being administered, one or more routes of administration may be used; for example, a first agent may be administered orally and a second agent may be administered intravenously. Methods of administration include injection for which the agents are provided in a nontoxic pharmaceutically acceptable carrier such as water, saline, Ringer's solution, dextrose solution, 5% human serum albumin, fixed oils, ethyl oleate, or liposomes. In some embodiments, local administration of the disclosed compounds can be used, for instance by applying the agent to cardiac tissue (intracardial administration). In some embodiments, sustained intra-cardial (or near-cardial) release of the pharmaceutical preparation that includes a therapeutically effective amount of the agent may be beneficial.

The compositions that include an agent can be formulated in unit dosage form suitable for individual administration of precise dosages. In addition, the compositions may be administered in a single dose or in a multiple dose schedule. A multiple dose schedule is one in which a primary course of treatment may be with more than one separate dose, for instance 1-10 doses, followed by other doses given at subsequent time intervals as needed to maintain or reinforce the action of the compositions. Treatment can involve daily or multi-daily doses of compound(s) over a period of a few days to months, or even years. Thus, the dosage regime will also, at least in part, be determined based on the particular needs of the subject to be treated and will be dependent upon the judgment of the administering practitioner.

Administration of the agent can also be accompanied by administration of other anti-arrhythmia agents or therapeutic treatments (such as surgical procedures, for example, installation of a pacemaker). For example, prior to, during, or following administration of a therapeutic amount of the agent, the subject can receive one or more additional therapies. Anti-arrhythmia agents and methods of their use are familiar to the person of ordinary skill in the art.

### A. Therapeutic Nucleic Acid Molecules

1. SIRTUIN Nucleic Acid Molecules

In some embodiments, the agent that increases the activity or expression of SIRTUIN includes a nucleic acid molecule encoding a SIRTUIN protein (e.g., SIRT1), for example, the agent can include an expression vector including the nucleic acid molecule encoding a SIRTUIN protein (e.g., SIRT1). SIRTUIN proteins are known to the person of ordinary skill in the art, and are disclosed herein. As disclosed herein, increasing the expression of SIRT1 leads to an increase in the Nav1.5 activity (such as an increase in I_{Na}) in the subject, thereby treating and/or inhibiting the cardiac arrhythmia, such as that caused by Brugada syndrome, in the subject.

Non-limiting examples of SIRTUIN proteins include yeast SIR2 protein (GENBANK™ Accession No. P53685; SEQ ID NO: 1), C. elegans Sir-2.1 (GENBANK™ Accession No. NP_{—}501912, SEQ ID NO: 2), human SIRT1 (GENBANK™ Accession Nos. NM_{—}012238 (encoding cDNA (SEQ ID NO: 3) and NP_{—}036370 (protein, SEQ ID NO: 4)), SIRT2, variant 1 (GENBANK™ Accession Nos. NM_{—}012237 (encoding cDNA; SEQ ID NO: 5) and NP_{—}036369 (protein, SEQ ID NO: 6); and SIRT2, variant 2 (GENBANK™ Accession Nos. NM_{—}030593 (encoding cDNA, SEQ ID NO: 7) and NP_{—}085096 (protein, SEQ ID NO: 8), and equivalents and fragments thereof. Other SIRTUIN family members include the four additional yeast Sir2-like genes termed “HST genes” (homologues of Sir two) HST1, HST2, HST3 and HST4, and the five other human homologues hSIRT3, hSIRT4, hSIRT5, hSIRT6 and hSIRT7 (see, e.g., Brachmann et al. Genes Dev. 9:2888, 1995 and Frye et al. BBRC 260:273, 1999).

SIRT1 protein include human SIRT1 (GENBANK™ Accession No. NM_{—}012238, SEQ ID NO: 3 and NP_{—}036370, SEQ ID NO: 4), human SIRT2, variant 1 (GENBANK™ Accession No. NM_{—}012237, SEQ ID NO: 5 (cDNA); and NP_{—}036369 (protein); SEQ ID NO: 6), and human SIRT2, variant 2 (GENBANK™ Accession No. NM_{—}030593 (cDNA), SEQ ID NO: 7; and NP_{—}085096 (protein), SEQ ID NO: 8) proteins, and equivalents and fragments thereof that retain biological activity (e.g., deacetylase activity). In another embodiment, a SIRT1 protein includes a polypeptide comprising a sequence consisting of, or consisting essentially of, the amino acid sequence set forth as one of SEQ ID NOs: 1, 2, 3, 4, 6, or 8. SIRT1 proteins include polypeptides comprising all or a portion of the amino acid sequence set forth sequence set forth as one of SEQ ID NOs: 1, 2, 3, 4, 6, or 8; the amino acid sequence set forth sequence set forth as one of SEQ ID NOs: 1, 2, 3, 4, 6, or 8 with 1 to about 2, 3, 5, 7, 10, or 15, conservative amino acid substitutions; or an amino acid sequence that is at least 95%, 96%, 97%, 98%, or 99% identical to one of SEQ ID NOs: 1, 2, 3, 4, 6, or 8, that retain biological activity (e.g., deacetylase activity).

2. Nav1.5 Nucleic Acid Molecules

In some embodiments, the agent includes an expression vector including a nucleic acid molecule encoding a mutated Nav1.5 alpha subunit that is non-acetylatable on lysine residue 1479 (for example, as described herein). This form of Nav1.5 alpha subunit, by being non-acetylatable, is not subject to down-regulation by endogenous acetylases, and functions as a constitutive sodium channel, increasing I_{Na}, thereby treating and/or inhibiting the cardiac arrhythmia, such as that caused by Brugada syndrome, in the subject.

Non-limiting examples of Nav1.5 proteins include cardiac sodium channels human sodium channel protein type 5 subunit alpha, isoforms a-f (corresponding to GENBANK™ Acc. Nos. NP_{—}932173 (SEQ ID NO: 10), NP_{—}000326.2 (SEQ ID NO: 12), NP_{—}001153632.1 (SEQ Id NO: 13), NP_{—}001092875.1 (SEQ Id NO: 14), NP_{—}001153632.1 (SEQ ID NO: 15), NP_{—}001153633.1 (SEQ ID NO: 16), respectively, each of which is incorporated by reference herein as present in GENBANK™ on May 4, 2013), and equivalents and fragments thereof that retain biological activity (e.g., sodium channel activity), and which include an amino acid substitution that eliminates the acetylation site at lysine 1479. In another embodiment, a Nav1.5 protein includes a polypeptide comprising a sequence consisting of, or consisting essentially of, the amino acid sequence set forth as any one of SEQ ID NOs: 10, 12, 13, 14, 15, or 16. Nav1.5 proteins include polypeptides comprising all or a portion of the amino acid sequence set forth sequence set forth as any one of SEQ ID NOs: 10, 12, 13, 14, 15, or 16; the amino acid sequence set forth sequence set forth as any one of SEQ ID NOs: 10, 12, 13, 14, 15, or 16 with 1 to about 2, 3, 5, 7, 10, or 15, conservative amino acid substitutions; or an amino acid sequence that is at least 95%, 96%, 97%, 98%, or 99% identical to the amino acid sequence set forth as any one of SEQ ID NOs: 10, 12, 13, 14, 15, or 16 that retain biological activity (e.g., sodium channel activity), and which include an amino acid substitution that eliminates the acetylation site at lysine 1479.

3. Additional Information Concerning Therapeutic Nucleic Acid Molecules

The therapeutic polypeptides of the present disclosure also can be administered as naked DNA encoding the polypeptide. To simplify the manipulation and handling of the nucleic acid encoding the peptide, the nucleic acid is generally inserted into a cassette, where it is operably linked to a promoter. Preferably, the promoter is capable of driving expression of the protein in cells of the desired target tissue. The selection of appropriate promoters can readily be accomplished. Preferably, the promoter is a high expression promoter, for example the 763-base-pair cytomegalovirus (CMV) promoter, the Rous sarcoma virus (RSV) promoter (Davis, et al., Hum. Gene. Ther. 4:151, 1993), or the MMT promoter.

Other elements that enhance expression also can be included, such as an enhancer or a system that results in high levels of expression, such as a tat gene or tar element. This cassette is inserted into a vector, for example, a plasmid vector such as pUC118, pBR322, or other known plasmid vector, that includes, for example, an E. coli origin of replication. See, Sambrook, et al., Molecular Cloning: A Laboratory Manual, Cold Spring Harbor Laboratory Press (1989). The plasmid vector may also include a selectable marker such as the β-lactamase gene for ampicillin resistance, provided that the marker polypeptide does not adversely affect the metabolism of the organism being treated. The cassette also can be bound to a nucleic acid binding moiety in a synthetic delivery system, such as the system disclosed in PCT publication WO 95/22618.

Optionally, the DNA may be used with a microdelivery vehicle such as cationic liposomes and adenoviral vectors. (For a review of the procedures for liposome preparation, targeting and delivery of contents, see Mannino and Gould-Fogerite, BioTechniques, 6:682, 1988); Feigner and Holm, Bethesda Res. Lab. Focus, 11(2):21, 1989); and Maurer, Bethesda Res. Lab. Focus, 11(2):25, 1989). Replication-defective recombinant adenoviral vectors can be produced in accordance with known techniques. (See Quantin, et al., Proc. Natl. Acad. Sci. USA, 89:2581-2584, 1992; Stratford-Perricadet, et al., J. Clin. Invest., 90:626-630, 1992; and Rosenfeld, et al., Cell, 68:143-155, 1992).

Once injected, the nucleic acid capable of expressing the desired protein is taken up and expressed by the cells of the tissue. Because the vectors containing the nucleic acid of interest are not normally incorporated into the genome of the cells, expression of the protein of interest takes place for only a limited time. Typically, the protein is only expressed in therapeutic levels for about two days to several weeks, preferably for about one to two weeks. Reinjection of the DNA can be utilized to provide additional periods of expression of the protein. If desired, use of a retrovirus vector to incorporate the heterologous DNA into the genome of the cells will increase the length of time during which the therapeutic polypeptide is expressed, from several weeks to indefinitely.

In some examples, a subject is administered DNA encoding a SIRTUIN protein (e.g., SIRT1), to provide in vivo production of the SIRTUIN protein (e.g., SIRT1), for example using the cellular machinery of the subject. Administration of nucleic acid constructs is well known in the art and taught, for example, in U.S. Pat. No. 5,643,578, and U.S. Pat. No. 5,593,972 and U.S. Pat. No. 5,817,637. U.S. Pat. No. 5,880,103 describes several methods of delivery of nucleic acids encoding therapeutic proteins to an organism. The methods include liposomal delivery of the nucleic acids. One approach to administration of nucleic acids is direct administration with plasmid DNA, such as with a mammalian expression plasmid. The nucleotide sequence encoding the SIRTUIN protein (e.g., SIRT1) can be placed under the control of a promoter to increase expression (for example a tissue specific promoter, such as a cardiac-specific promoter).

In another approach to using nucleic acids, SIRTUIN protein (e.g., SIRT1) can also be expressed by attenuated viral hosts or vectors or bacterial vectors. Recombinant vaccinia virus, adeno-associated virus (AAV), herpes virus, retrovirus, cytomegalovirus or other viral vectors can be used to express the nucleic acid. For example, vaccinia vectors and methods useful protocols are described in U.S. Pat. No. 4,722,848. BCG (Bacillus Calmette Guerin) provides another vector for expression of the disclosed antibodies (see Stover, Nature 351:456-460, 1991).

In one embodiment, a nucleic acid encoding a SIRTUIN protein (e.g., SIRT1) is introduced directly into cells. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter.

Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

### B. SIRT1 Activators

In some embodiments, the agent that increases the activity or expression of SIRT1 is a SIRT1 activator. Numerous SIRT1 activators are known to the person of ordinary skill in the art, and are further described herein (see below). In one embodiment, the SIRT1 activator is resveratrol. As disclosed herein, increasing the activity of SIRT1 leads to an increase in the Nav1.5 activity (such as an increase in I_{Na}) in the subject, thereby treating and/or inhibiting the cardiac arrhythmia, such as that caused by Brugada syndrome, in the subject.

Numerous SIRT1 activators are known to the person of ordinary skill in the art. For example SIRT1 activators are described in U.S. Patent Publications 20130085155; 20120197013; 20120165330; 20120108585; 20120022254; 20110306612; 20110306609; 20110263564; 20110257174; 20110152254; 20110130387; 20110077248; 20110039847; 20110015192; 20110009496; 20100215632; 20090163476; 20090105246; 20090099170; 20090069301; 20090012080; 20080249103; 20070043050; 20070037865; 20070037827; 20070037809; 8,343,997; 8,268,862; 8,247,565; 8,178,536; 8,163,908; 8,093,401; 8,088,928; 8,044,198; 7,998,974; 7,893,086; 7,855,289; 7,829,556; 7,345,178, each of which is incorporated by reference herein in its entirety. SIRT1 activators are further described in Dai et al., J Biol Chem, 285 (43): 32695-32703, 2010, which is incorporated by reference herein in its entirety. Additional SIRT1 activators are provided as Formulas I-XXXVIII of U.S. Pat. No. 8,044,198, which is incorporated herein by reference.

In some embodiments, an activator of Formulas I-XXXVIII of U.S. Pat. No. 8,044,198 increases Nav1.5 activation. In some embodiments, an activator of Formulas I-XXXVIII increases the expression or activity of SRIT1 and increases Nav1.5 activation.

In some embodiments, the SIRT1 activator comprises Structure I:

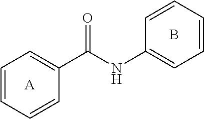

or a salt thereof, wherein:

Ring A is optionally substituted, fused to another ring or both; and

Ring B is substituted with at least one carboxy, substituted or unsubstituted arylcarboxamine, substituted or unsubstituted heteroaryl group, substituted or unsubstituted heterocyclylcarbonylethenyl, or polycyclic aryl group or is fused to an aryl ring and is optionally substituted by one or more additional groups.

In further embodiments, the SIRT1 activator comprises Structure II:

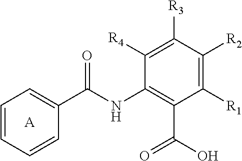

or a salt thereof, where

Ring A is optionally substituted;
R₁, R₂, R₃, and R₄ are independently selected from the group consisting of —H, halogen, —OR₅, —CN, —CO₂R₅, —OCOR₅, —OCO₂R₅, —C(O)NR₅R₆,
   —OC(O)NR₅R₆, —C(O)R₅, —COR_{5S}, —SR₅, —OSO₃H, —S(O)ₙR₅, —S(O)ₙOR₅,
   —S(O)ₙNR₅R₆, R₅ and R₆ are independently —H, a substituted or unsubstituted alkyl group, a substituted or unsubstituted aryl group, or a substituted or unsubstituted heterocyclic group; and
n is 1 or 2.

In one specific non-limiting example, the SIRT1 activator is N-[2-[3-(piperazin-1-ylmethyl)imidazo[2,1-b][1,3]thiazol-6-yl]phenyl]quinoxaline-2-carboxamide:

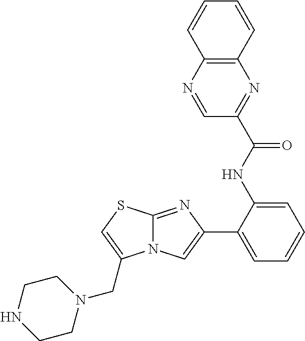 [SMILES: O=C(Nc1ccccc1-c1cn2c(CN3CCNCC3)csc2n1)c1cnc2ccccc2n1]

Additional SIRT1 activators for use in the disclosed methods are provided in Table 4 of U.S. Pat. No. 8,044,198, which is incorporated herein by reference. Compounds 1-160 of Table 4 of U.S. Pat. No. 8,044,198 are listed below. Compounds 161-745 as provided in Table 4 of U.S. Pat. No. 8,044,198 (incorporated by referenced herein) are also of use in the disclosed methods. Methods for producing these compounds are familiar to the person of ordinary skill in the art, and can be found for example, in U.S. Pat. No. 8,044,198. In some embodiments, the disclosed methods of treating or inhibiting cardiac arrhythmia (such as treating or inhibiting Brugada syndrome) include administering a therapeutically effective amount of one or more of the compounds listed as compounds 1-745 as provided in Table 4 of U.S. Pat. No. 8,044,198, such as administering a therapeutically effective amount of compound 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, or 745 as provided in Table 4 of U.S. Pat. No. 8,044,198 to a subject with cardiac arrhythmia, such as a subject with Brugada syndrome.

In some embodiments, the disclosed methods of treating or inhibiting cardiac arrhythmia (such as treating or inhibiting Brugada syndrome) include administering a therapeutically effective amount of one or more of a small molecule, such as one of the pharmaceutical compounds listed as compounds I-160 (methods of making these compounds are provided U.S. Pat. No. 8,044,198) as provided in the following table:

TABLE 2
SIRT1 activators

| | |
|---|---|
| 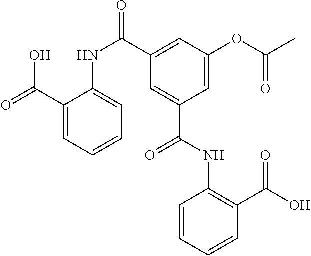 [SMILES: CC(=O)Oc1cc(C(=O)Nc2ccccc2C(=O)O)cc(C(=O)Nc2ccccc2C(=O)O)c1] | 1 |
| 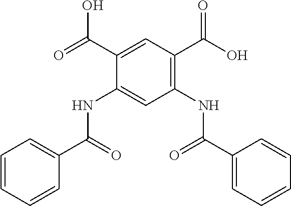 [SMILES: O=C(Nc1cc(NC(=O)c2ccccc2)c(C(=O)O)cc1C(=O)O)c1ccccc1] | 2 |
| 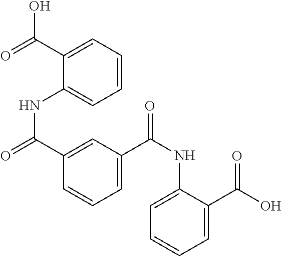 [SMILES: O=C(Nc1ccccc1C(=O)O)c1cccc(C(=O)Nc2ccccc2C(=O)O)c1] | 3 |
| 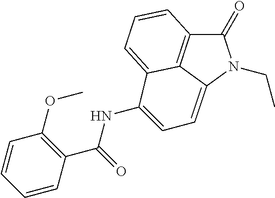 [SMILES: CCN1C(=O)c2cccc3c(NC(=O)c4ccccc4OC)ccc1c23] | 4 |
| 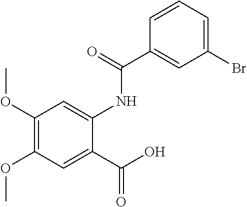 [SMILES: COc1cc(NC(=O)c2cccc(Br)c2)c(C(=O)O)cc1OC] | 5 |
| 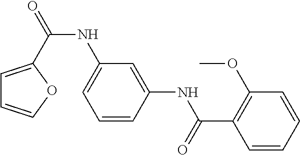 [SMILES: COc1ccccc1C(=O)Nc1cccc(NC(=O)c2ccco2)c1] | 6 |
| 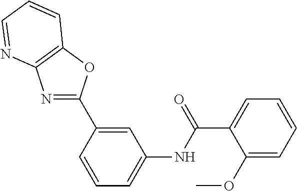 [SMILES: COc1ccccc1C(=O)Nc1cccc(-c2nc3ncccc3o2)c1] | 7 |
| 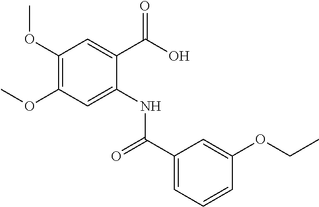 [SMILES: CCOc1cccc(C(=O)Nc2cc(OC)c(OC)cc2C(=O)O)c1] | 8 |
| 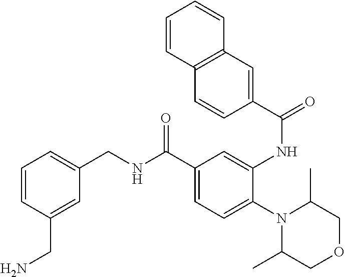 [SMILES: CC1COCC(C)N1c1ccc(C(=O)NCc2cccc(CN)c2)cc1NC(=O)c1ccc2ccccc2c1] | 9 |
| 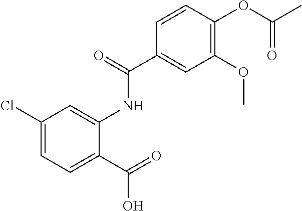 [SMILES: COc1cc(C(=O)Nc2cc(Cl)ccc2C(=O)O)ccc1OC(C)=O] | 10 |
| 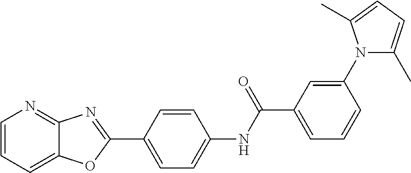 [SMILES: Cc1ccc(C)n1-c1cccc(C(=O)Nc2ccc(-c3nc4ncccc4o3)cc2)c1] | 19 |
| 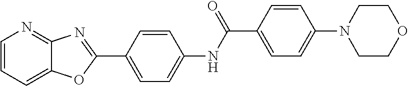 [SMILES: O=C(Nc1ccc(-c2nc3ncccc3o2)cc1)c1ccc(N2CCOCC2)cc1] | 20 |
| 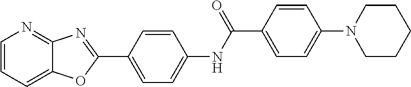 [SMILES: O=C(Nc1ccc(-c2nc3ncccc3o2)cc1)c1ccc(N2CCCCC2)cc1] | 21 |
| 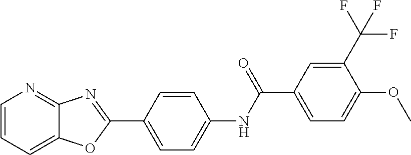 [SMILES: COc1ccc(C(=O)Nc2ccc(-c3nc4ncccc4o3)cc2)cc1C(F)(F)F] | 22 |
| 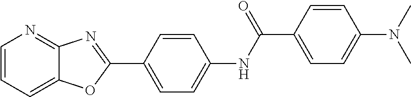 [SMILES: CN(C)c1ccc(C(=O)Nc2ccc(-c3nc4ncccc4o3)cc2)cc1] | 24 |
| 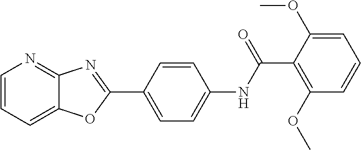 [SMILES: COc1cccc(OC)c1C(=O)Nc1ccc(-c2nc3ncccc3o2)cc1] | 27 |
| 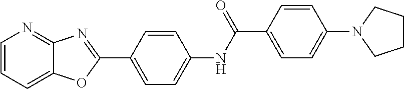 [SMILES: O=C(Nc1ccc(-c2nc3ncccc3o2)cc1)c1ccc(N2CCCC2)cc1] | 29 |
| 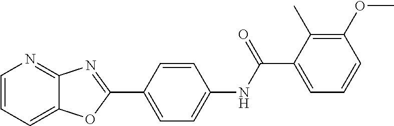 [SMILES: COc1cccc(C(=O)Nc2ccc(-c3nc4ncccc4o3)cc2)c1C] | 31 |
| 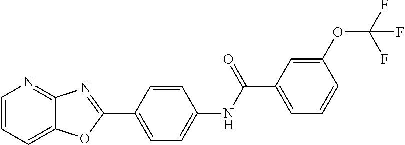 [SMILES: O=C(Nc1ccc(-c2nc3ncccc3o2)cc1)c1cccc(OC(F)(F)F)c1] | 32 |
| 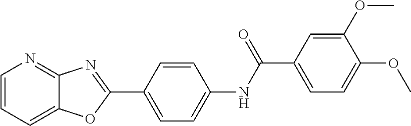 [SMILES: COc1ccc(C(=O)Nc2ccc(-c3nc4ncccc4o3)cc2)cc1OC] | 33 |
| 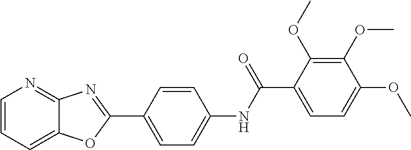 [SMILES: COc1ccc(C(=O)Nc2ccc(-c3nc4ncccc4o3)cc2)c(OC)c1OC] | 34 |
| 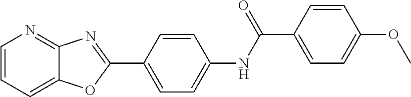 [SMILES: COc1ccc(C(=O)Nc2ccc(-c3nc4ncccc4o3)cc2)cc1] | 35 |
| 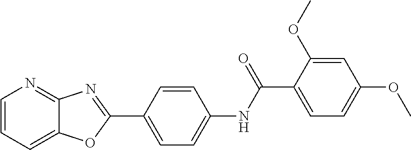 [SMILES: COc1ccc(C(=O)Nc2ccc(-c3nc4ncccc4o3)cc2)c(OC)c1] | 36 |
| 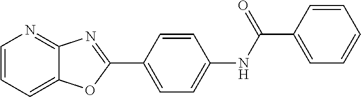 [SMILES: O=C(Nc1ccc(-c2nc3ncccc3o2)cc1)c1ccccc1] | 37 |
| 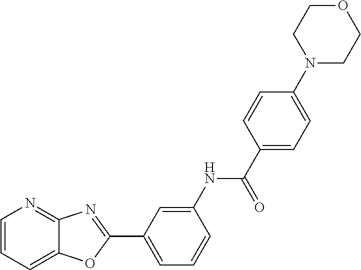 [SMILES: O=C(Nc1cccc(-c2nc3ncccc3o2)c1)c1ccc(N2CCOCC2)cc1] | 38 |
| 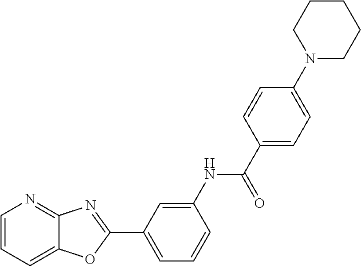 [SMILES: O=C(Nc1cccc(-c2nc3ncccc3o2)c1)c1ccc(N2CCCCC2)cc1] | 39 |
| 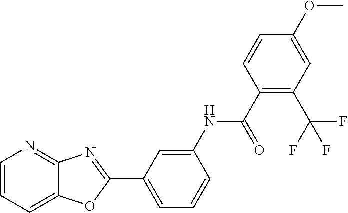 [SMILES: COc1ccc(C(=O)Nc2cccc(-c3nc4ncccc4o3)c2)c(C(F)(F)F)c1] | 40 |
| 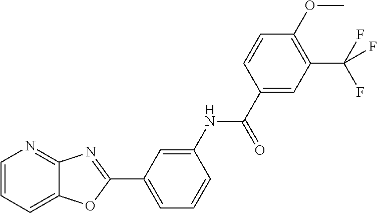 [SMILES: COc1ccc(C(=O)Nc2cccc(-c3nc4ncccc4o3)c2)cc1C(F)(F)F] | 41 |
| 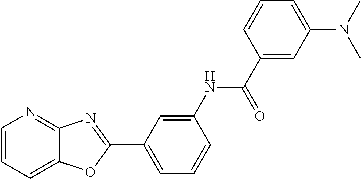 [SMILES: CN(C)c1cccc(C(=O)Nc2cccc(-c3nc4ncccc4o3)c2)c1] | 42 |
| 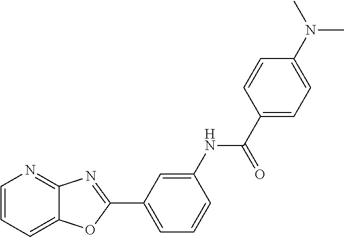 [SMILES: CN(C)c1ccc(C(=O)Nc2cccc(-c3nc4ncccc4o3)c2)cc1] | 43 |
| 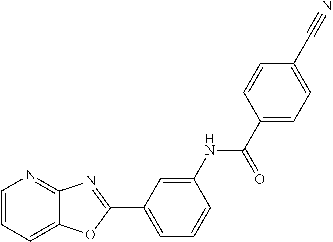 [SMILES: N#Cc1ccc(C(=O)Nc2cccc(-c3nc4ncccc4o3)c2)cc1] | 45 |
| 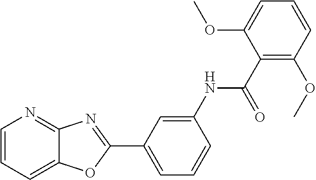 [SMILES: COc1cccc(OC)c1C(=O)Nc1cccc(-c2nc3ncccc3o2)c1] | 46 |
| 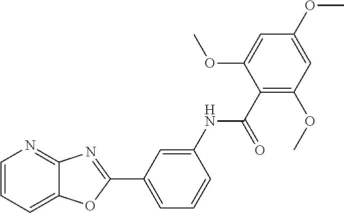 [SMILES: COc1cc(OC)c(C(=O)Nc2cccc(-c3nc4ncccc4o3)c2)c(OC)c1] | 48 |
| 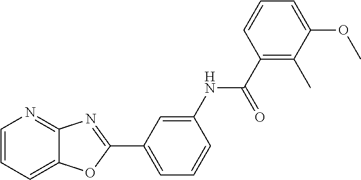 [SMILES: COc1cccc(C(=O)Nc2cccc(-c3nc4ncccc4o3)c2)c1C] | 49 |
| 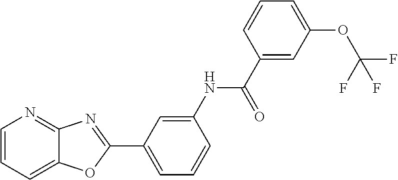 [SMILES: O=C(Nc1cccc(-c2nc3ncccc3o2)c1)c1cccc(OC(F)(F)F)c1] | 50 |
| 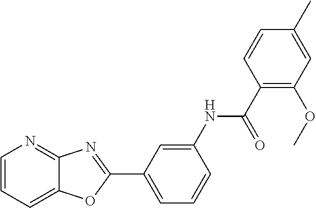 [SMILES: COc1cc(C)ccc1C(=O)Nc1cccc(-c2nc3ncccc3o2)c1] | 51 |
| 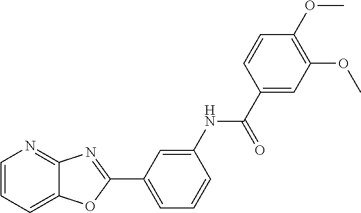 [SMILES: COc1ccc(C(=O)Nc2cccc(-c3nc4ncccc4o3)c2)cc1OC] | 52 |
| 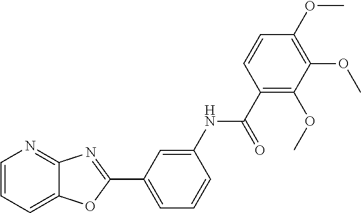 [SMILES: COc1ccc(C(=O)Nc2cccc(-c3nc4ncccc4o3)c2)c(OC)c1OC] | 53 |
| 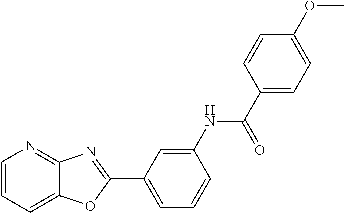 [SMILES: COc1ccc(C(=O)Nc2cccc(-c3nc4ncccc4o3)c2)cc1] | 54 |
| 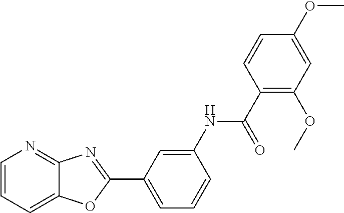 [SMILES: COc1ccc(C(=O)Nc2cccc(-c3nc4ncccc4o3)c2)c(OC)c1] | 55 |
| 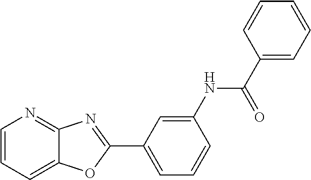 [SMILES: O=C(Nc1cccc(-c2nc3ncccc3o2)c1)c1ccccc1] | 56 |
| 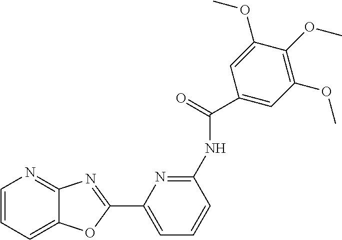 [SMILES: COc1cc(C(=O)Nc2cccc(-c3nc4ncccc4o3)n2)cc(OC)c1OC] | 57 |
| 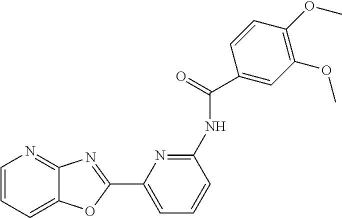 [SMILES: COc1ccc(C(=O)Nc2cccc(-c3nc4ncccc4o3)n2)cc1OC] | 58 |
| 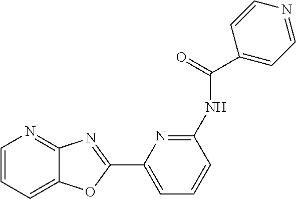 [SMILES: O=C(Nc1cccc(-c2nc3ncccc3o2)n1)c1ccncc1] | 59 |
| 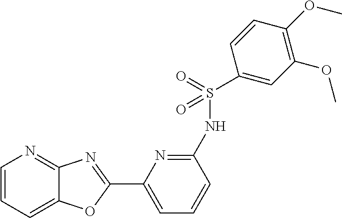 [SMILES: COc1ccc(S(=O)(=O)Nc2cccc(-c3nc4ncccc4o3)n2)cc1OC] | 60 |
| 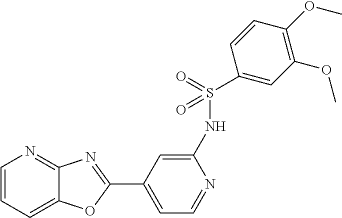 [SMILES: COc1ccc(S(=O)(=O)Nc2cc(-c3nc4ncccc4o3)ccn2)cc1OC] | 61 |
| 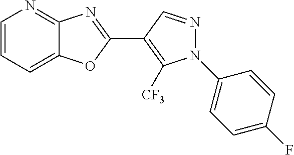 [SMILES: Fc1ccc(-n2ncc(-c3nc4ncccc4o3)c2C(F)(F)F)cc1] | 62 |
| 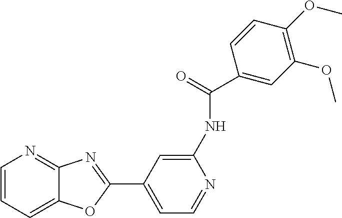 [SMILES: COc1ccc(C(=O)Nc2cc(-c3nc4ncccc4o3)ccn2)cc1OC] | 63 |
| 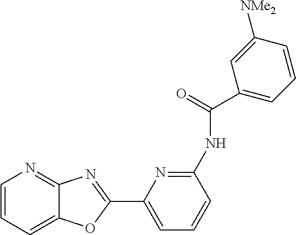 [SMILES: CN(C)c1cccc(C(=O)Nc2cccc(-c3nc4ncccc4o3)n2)c1] | 64 |
| 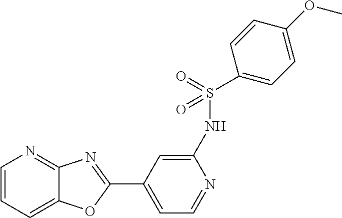 [SMILES: COc1ccc(S(=O)(=O)Nc2cc(-c3nc4ncccc4o3)ccn2)cc1] | 65 |
| 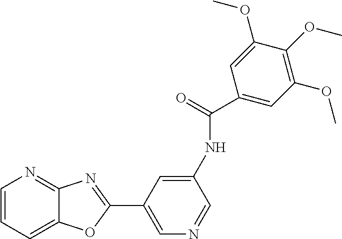 [SMILES: COc1cc(C(=O)Nc2cncc(-c3nc4ncccc4o3)c2)cc(OC)c1OC] | 66 |
| 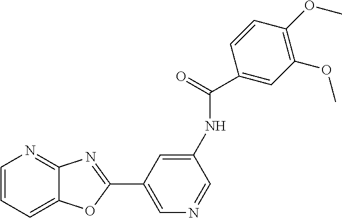 [SMILES: COc1ccc(C(=O)Nc2cncc(-c3nc4ncccc4o3)c2)cc1OC] | 67 |
| 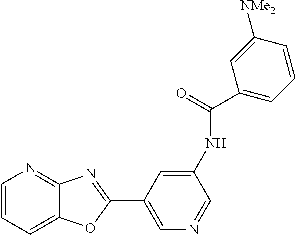 [SMILES: CN(C)c1cccc(C(=O)Nc2cncc(-c3nc4ncccc4o3)c2)c1] | 68 |
| 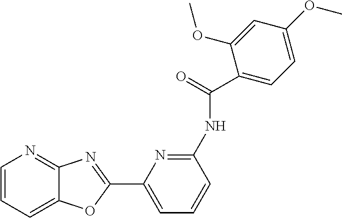 [SMILES: COc1ccc(C(=O)Nc2cccc(-c3nc4ncccc4o3)n2)c(OC)c1] | 69 |
| 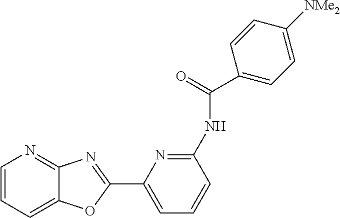 [SMILES: CN(C)c1ccc(C(=O)Nc2cccc(-c3nc4ncccc4o3)n2)cc1] | 70 |
| 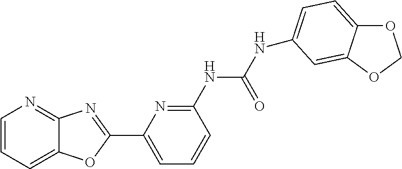 [SMILES: O=C(Nc1ccc2c(c1)OCO2)Nc1cccc(-c2nc3ncccc3o2)n1] | 71 |
| 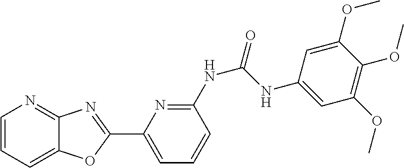 [SMILES: COc1cc(NC(=O)Nc2cccc(-c3nc4ncccc4o3)n2)cc(OC)c1OC] | 72 |
| 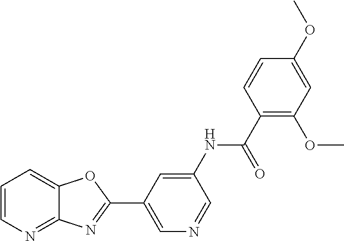 [SMILES: COc1ccc(C(=O)Nc2cncc(-c3nc4ncccc4o3)c2)c(OC)c1] | 73 |
| 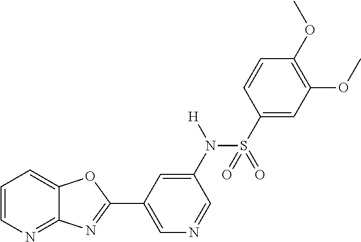 [SMILES: COc1ccc(S(=O)(=O)Nc2cncc(-c3nc4ncccc4o3)c2)cc1OC] | 74 |
| 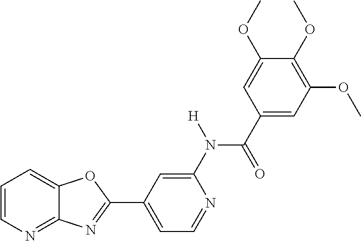 [SMILES: COc1cc(C(=O)Nc2cc(-c3nc4ncccc4o3)ccn2)cc(OC)c1OC] | 75 |
| 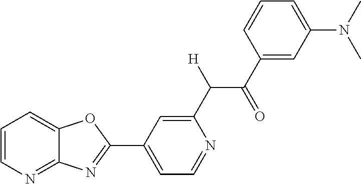 [SMILES: CN(C)c1cccc(C(=O)Cc2cc(-c3nc4ncccc4o3)ccn2)c1] | 76 |
| 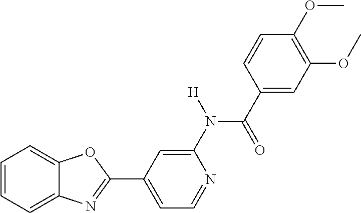 [SMILES: COc1ccc(C(=O)Nc2cc(-c3nc4ccccc4o3)ccn2)cc1OC] | 77 |
| 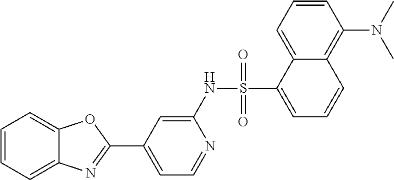 [SMILES: CN(C)c1cccc2c(S(=O)(=O)Nc3cc(-c4nc5ccccc5o4)ccn3)cccc12] | 78 |
| 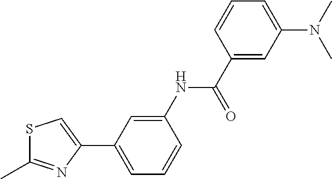 | 79 |
| 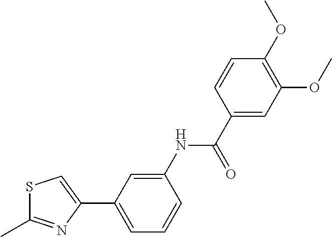 | 80 |
| 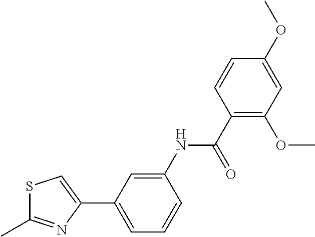 | 81 |
| 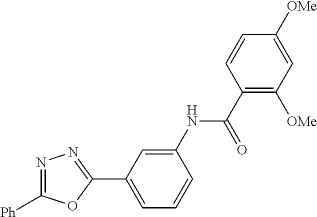 [SMILES: COc1ccc(C(=O)Nc2cccc(-c3nnc(-c4ccccc4)o3)c2)c(OC)c1] | 82 |
| 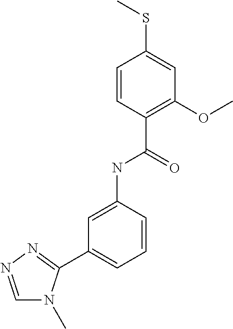 [SMILES: COc1cc(SC)ccc1C(=O)Nc1cccc(-c2nncn2C)c1] | 83 |
| 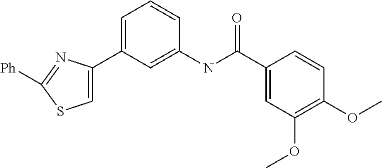 [SMILES: COc1ccc(C(=O)Nc2cccc(-c3csc(-c4ccccc4)n3)c2)cc1OC] | 84 |
| 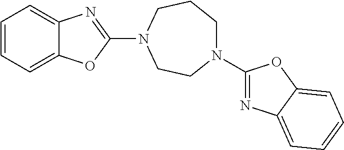 [SMILES: c1ccc2oc(N3CCCN(c4nc5ccccc5o4)CC3)nc2c1] | 85 |
| 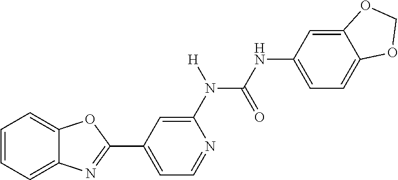 [SMILES: O=C(Nc1ccc2c(c1)OCO2)Nc1cc(-c2nc3ccccc3o2)ccn1] | 86 |
| 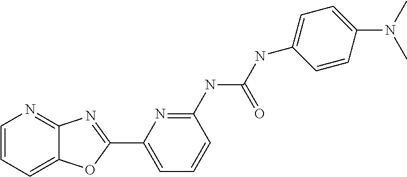 [SMILES: CN(C)c1ccc(NC(=O)Nc2cccc(-c3nc4ncccc4o3)n2)cc1] | 87 |
| 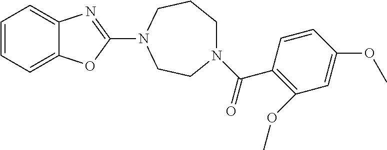 [SMILES: COc1ccc(C(=O)N2CCCN(c3nc4ccccc4o3)CC2)c(OC)c1] | 88 |
| 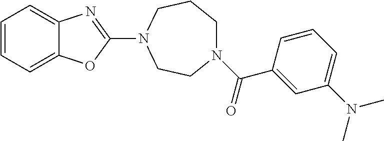 [SMILES: CN(C)c1cccc(C(=O)N2CCCN(c3nc4ccccc4o3)CC2)c1] | 89 |
| 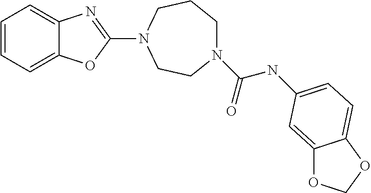 [SMILES: O=C(Nc1ccc2c(c1)OCO2)N1CCCN(c2nc3ccccc3o2)CC1] | 90 |
| 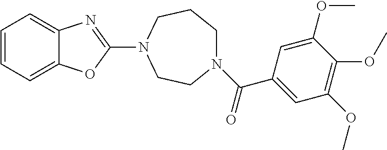 [SMILES: COc1cc(C(=O)N2CCCN(c3nc4ccccc4o3)CC2)cc(OC)c1OC] | 91 |
| 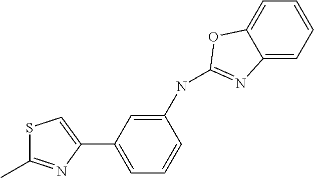 [SMILES: Cc1nc(-c2cccc(Nc3nc4ccccc4o3)c2)cs1] | 92 |
| 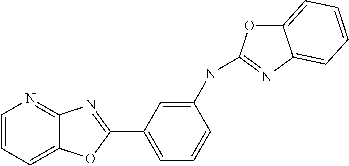 [SMILES: c1cc(Nc2nc3ccccc3o2)cc(-c2nc3ncccc3o2)c1] | 93 |
| 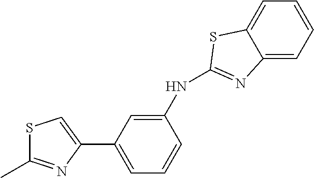 [SMILES: Cc1nc(-c2cccc(Nc3nc4ccccc4s3)c2)cs1] | 94 |
| 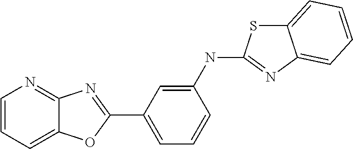 [SMILES: c1cc(Nc2nc3ccccc3s2)cc(-c2nc3ncccc3o2)c1] | 95 |
| 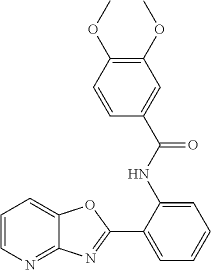 [SMILES: COc1ccc(C(=O)Nc2ccccc2-c2nc3ncccc3o2)cc1OC] | 96 |
| 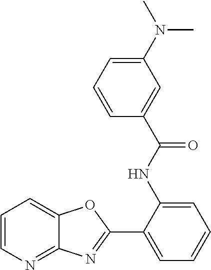 [SMILES: CN(C)c1cccc(C(=O)Nc2ccccc2-c2nc3ncccc3o2)c1] | 97 |
| 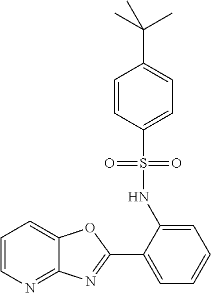 [SMILES: CC(C)(C)c1ccc(S(=O)(=O)Nc2ccccc2-c2nc3ncccc3o2)cc1] | 98 |
| 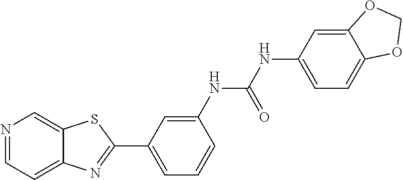 [SMILES: O=C(Nc1cccc(-c2nc3ccncc3s2)c1)Nc1ccc2c(c1)OCO2] | 99 |
| 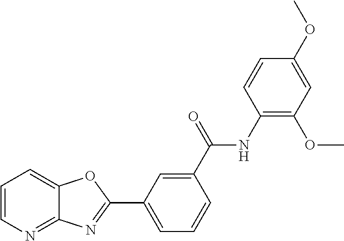 [SMILES: COc1ccc(NC(=O)c2cccc(-c3nc4ncccc4o3)c2)c(OC)c1] | 100 |
| 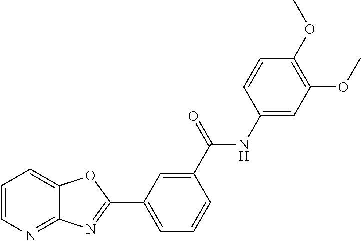 [SMILES: COc1ccc(NC(=O)c2cccc(-c3nc4ncccc4o3)c2)cc1OC] | 101 |
| 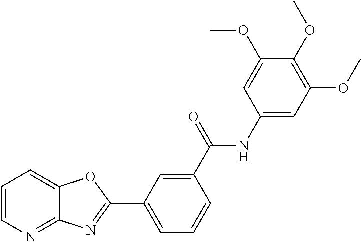 [SMILES: COc1cc(NC(=O)c2cccc(-c3nc4ncccc4o3)c2)cc(OC)c1OC] | 102 |
| 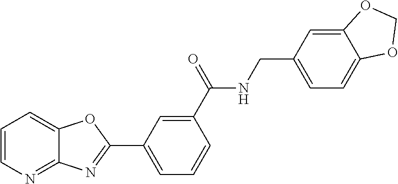 [SMILES: O=C(NCc1ccc2c(c1)OCO2)c1cccc(-c2nc3ncccc3o2)c1] | 103 |
| 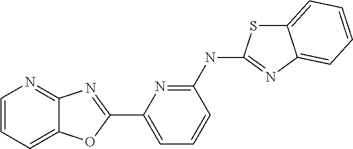 [SMILES: c1cc(Nc2nc3ccccc3s2)nc(-c2nc3ncccc3o2)c1] | 104 |
| 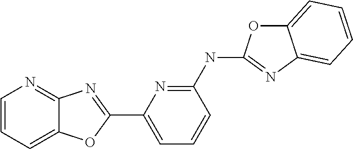 [SMILES: c1cc(Nc2nc3ccccc3o2)nc(-c2nc3ncccc3o2)c1] | 105 |
| 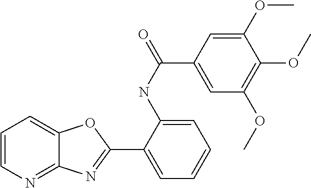 [SMILES: COc1cc(C(=O)Nc2ccccc2-c2nc3ncccc3o2)cc(OC)c1OC] | 106 |
| 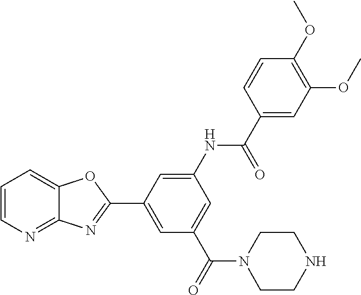 [SMILES: COc1ccc(C(=O)Nc2cc(C(=O)N3CCNCC3)cc(-c3nc4ncccc4o3)c2)cc1OC] | 107 |
| 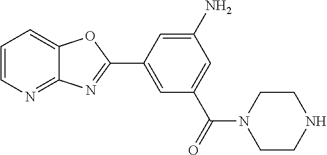 [SMILES: Nc1cc(C(=O)N2CCNCC2)cc(-c2nc3ncccc3o2)c1] | 108 |
| 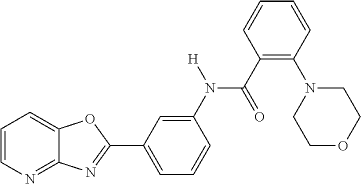 [SMILES: O=C(Nc1cccc(-c2nc3ncccc3o2)c1)c1ccccc1N1CCOCC1] | 109 |
| 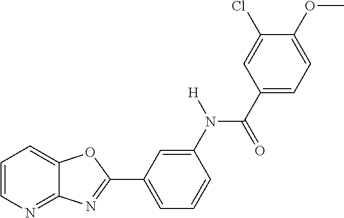 [SMILES: COc1ccc(C(=O)Nc2cccc(-c3nc4ncccc4o3)c2)cc1Cl] | 110 |
| 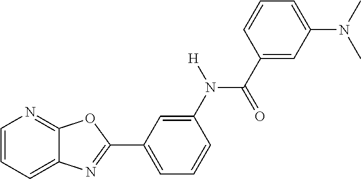 [SMILES: CN(C)c1cccc(C(=O)Nc2cccc(-c3nc4cccnc4o3)c2)c1] | 111 |
| 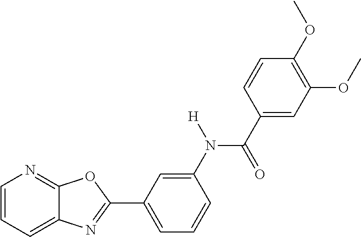 [SMILES: COc1ccc(C(=O)Nc2cccc(-c3nc4cccnc4o3)c2)cc1OC] | 112 |
| 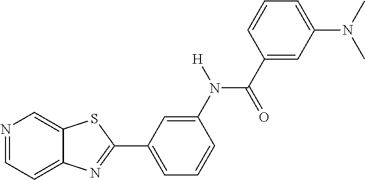 [SMILES: CN(C)c1cccc(C(=O)Nc2cccc(-c3nc4ccncc4s3)c2)c1] | 113 |
| 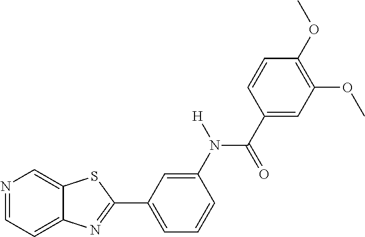 [SMILES: COc1ccc(C(=O)Nc2cccc(-c3nc4ccncc4s3)c2)cc1OC] | 114 |
| 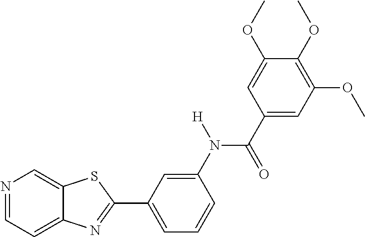 [SMILES: COc1cc(C(=O)Nc2cccc(-c3nc4ccncc4s3)c2)cc(OC)c1OC] | 115 |
| 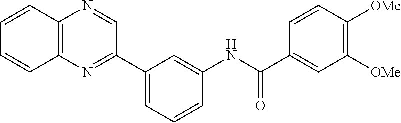 [SMILES: COc1ccc(C(=O)Nc2cccc(-c3cnc4ccccc4n3)c2)cc1OC] | 116 |
| 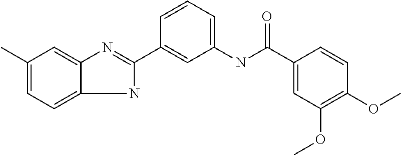 | 117 |
| 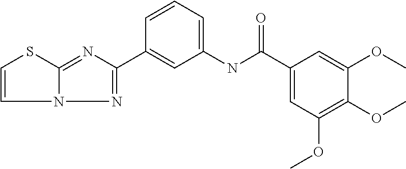 [SMILES: COc1cc(C(=O)Nc2cccc(-c3nc4sccn4n3)c2)cc(OC)c1OC] | 118 |
| 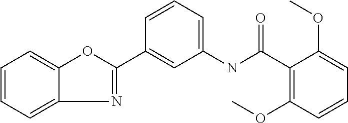 [SMILES: COc1cccc(OC)c1C(=O)Nc1cccc(-c2nc3ccccc3o2)c1] | 119 |
| 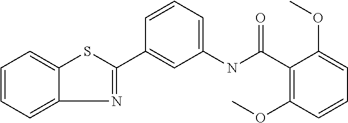 [SMILES: COc1cccc(OC)c1C(=O)Nc1cccc(-c2nc3ccccc3s2)c1] | 120 |
| 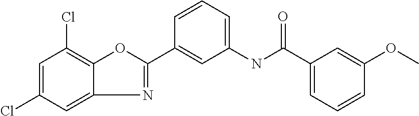 [SMILES: COc1cccc(C(=O)Nc2cccc(-c3nc4cc(Cl)cc(Cl)c4o3)c2)c1] | 121 |
| 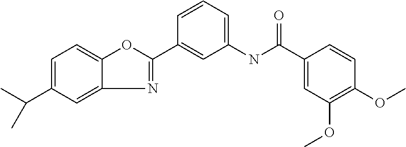 [SMILES: COc1ccc(C(=O)Nc2cccc(-c3nc4cc(C(C)C)ccc4o3)c2)cc1OC] | 122 |
| 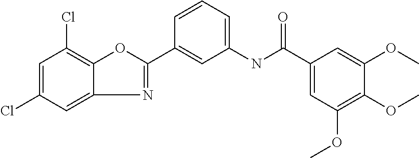 [SMILES: COc1cc(C(=O)Nc2cccc(-c3nc4cc(Cl)cc(Cl)c4o3)c2)cc(OC)c1OC] | 123 |
| 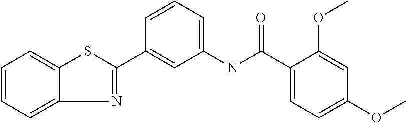 [SMILES: COc1ccc(C(=O)Nc2cccc(-c3nc4ccccc4s3)c2)c(OC)c1] | 124 |
| 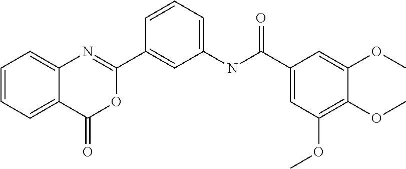 [SMILES: COc1cc(C(=O)Nc2cccc(-c3nc4ccccc4c(=O)o3)c2)cc(OC)c1OC] | 125 |
| 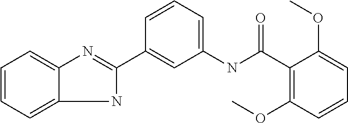 [SMILES: COc1cccc(OC)c1C(=O)Nc1cccc(-c2nc3ccccc3[nH]2)c1] | 126 |
| 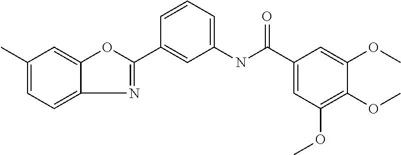 | 127 |
| 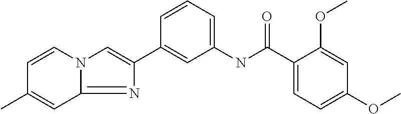 | 128 |
| 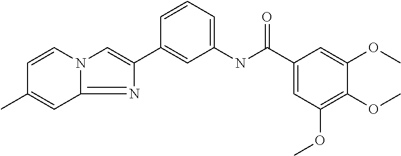 | 129 |
| 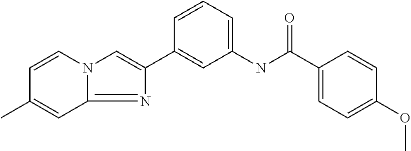 | 130 |
| 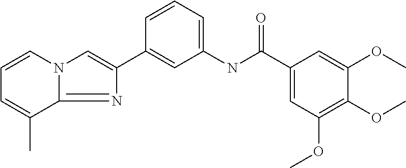 [SMILES: COc1cc(C(=O)Nc2cccc(-c3cn4cccc(C)c4n3)c2)cc(OC)c1OC] | 131 |
| 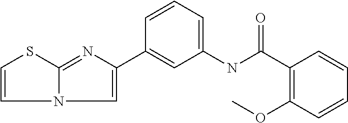 [SMILES: COc1ccccc1C(=O)Nc1cccc(-c2cn3ccsc3n2)c1] | 132 |
| 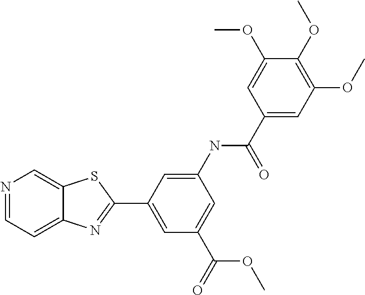 [SMILES: COC(=O)c1cc(NC(=O)c2cc(OC)c(OC)c(OC)c2)cc(-c2nc3ccncc3s2)c1] | 133 |
| 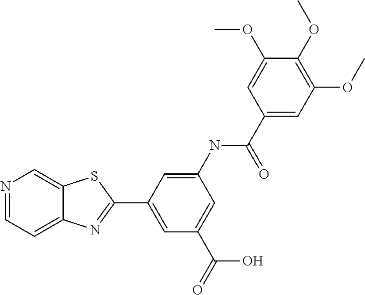 [SMILES: COc1cc(C(=O)Nc2cc(C(=O)O)cc(-c3nc4ccncc4s3)c2)cc(OC)c1OC] | 134 |
| 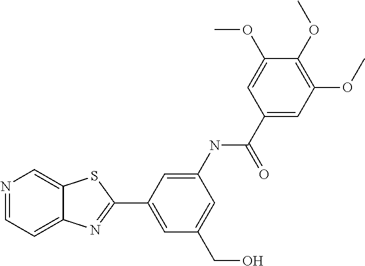 [SMILES: COc1cc(C(=O)Nc2cc(CO)cc(-c3nc4ccncc4s3)c2)cc(OC)c1OC] | 135 |
| 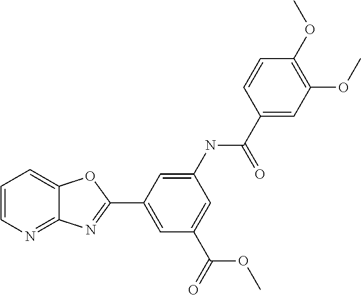 [SMILES: COC(=O)c1cc(NC(=O)c2ccc(OC)c(OC)c2)cc(-c2nc3ncccc3o2)c1] | 136 |
| 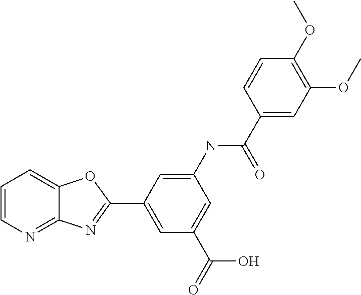 [SMILES: COc1ccc(C(=O)Nc2cc(C(=O)O)cc(-c3nc4ncccc4o3)c2)cc1OC] | 137 |
| 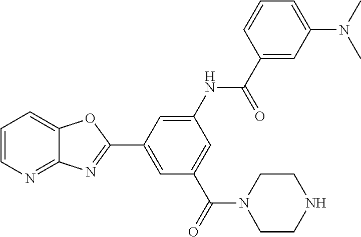 [SMILES: CN(C)c1cccc(C(=O)Nc2cc(C(=O)N3CCNCC3)cc(-c3nc4ncccc4o3)c2)c1] | 138 |
| 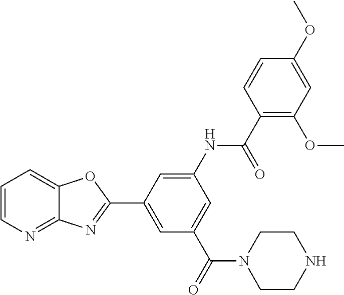 [SMILES: COc1ccc(C(=O)Nc2cc(C(=O)N3CCNCC3)cc(-c3nc4ncccc4o3)c2)c(OC)c1] | 139 |
| 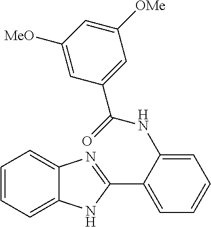 [SMILES: COc1cc(OC)cc(C(=O)Nc2ccccc2-c2nc3ccccc3[nH]2)c1] | 141 |
| 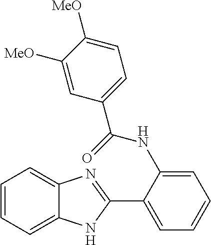 [SMILES: COc1ccc(C(=O)Nc2ccccc2-c2nc3ccccc3[nH]2)cc1OC] | 142 |
| 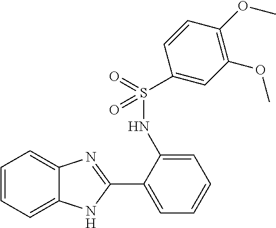 [SMILES: COc1ccc(S(=O)(=O)Nc2ccccc2-c2nc3ccccc3[nH]2)cc1OC] | 143 |
| 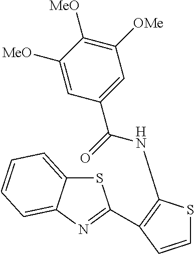 [SMILES: COc1cc(C(=O)Nc2sccc2-c2nc3ccccc3s2)cc(OC)c1OC] | 144 |
| 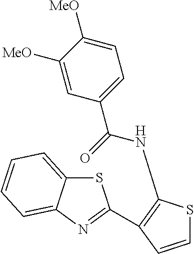 [SMILES: COc1ccc(C(=O)Nc2sccc2-c2nc3ccccc3s2)cc1OC] | 145 |
| 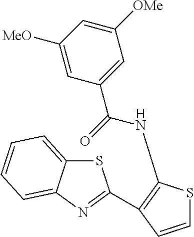 [SMILES: COc1cc(OC)cc(C(=O)Nc2sccc2-c2nc3ccccc3s2)c1] | 146 |
| 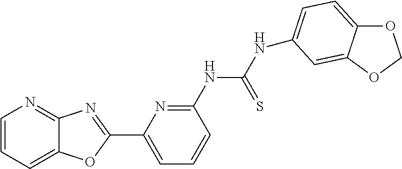 [SMILES: S=C(Nc1ccc2c(c1)OCO2)Nc1cccc(-c2nc3ncccc3o2)n1] | 147 |
| 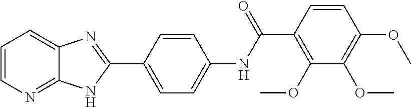 [SMILES: COc1ccc(C(=O)Nc2ccc(-c3nc4cccnc4[nH]3)cc2)c(OC)c1OC] | 148 |
| 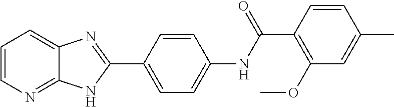 [SMILES: COc1cc(C)ccc1C(=O)Nc1ccc(-c2nc3cccnc3[nH]2)cc1] | 149 |
| 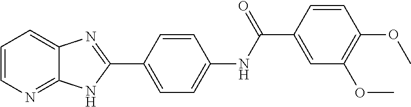 [SMILES: COc1ccc(C(=O)Nc2ccc(-c3nc4cccnc4[nH]3)cc2)cc1OC] | 150 |
| 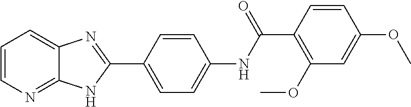 [SMILES: COc1ccc(C(=O)Nc2ccc(-c3nc4cccnc4[nH]3)cc2)c(OC)c1] | 151 |
| 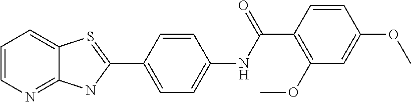 | 152 |
| 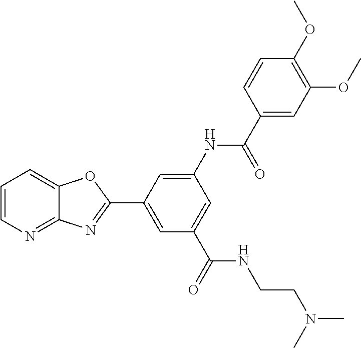 [SMILES: COc1ccc(C(=O)Nc2cc(C(=O)NCCN(C)C)cc(-c3nc4ncccc4o3)c2)cc1OC] | 153 |
| 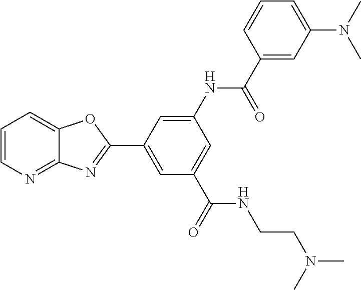 [SMILES: CN(C)CCNC(=O)c1cc(NC(=O)c2cccc(N(C)C)c2)cc(-c2nc3ncccc3o2)c1] | 154 |
| 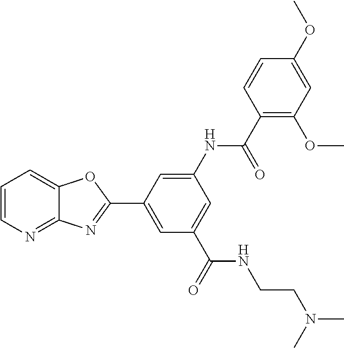 [SMILES: COc1ccc(C(=O)Nc2cc(C(=O)NCCN(C)C)cc(-c3nc4ncccc4o3)c2)c(OC)c1] | 155 |
| 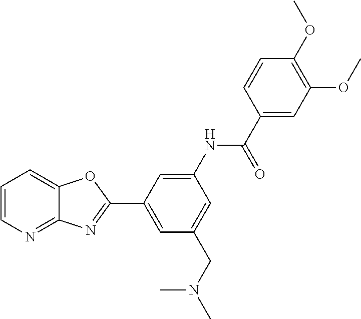 [SMILES: COc1ccc(C(=O)Nc2cc(CN(C)C)cc(-c3nc4ncccc4o3)c2)cc1OC] | 156 |
| 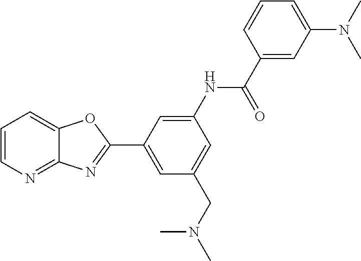 [SMILES: CN(C)Cc1cc(NC(=O)c2cccc(N(C)C)c2)cc(-c2nc3ncccc3o2)c1] | 157 |
| 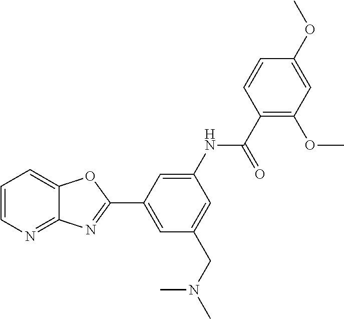 [SMILES: COc1ccc(C(=O)Nc2cc(CN(C)C)cc(-c3nc4ncccc4o3)c2)c(OC)c1] | 158 |
| 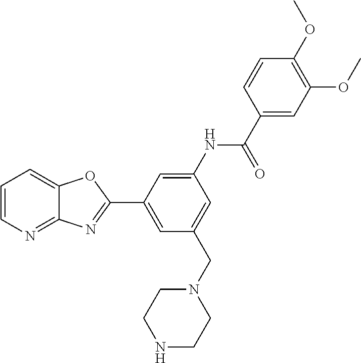 [SMILES: COc1ccc(C(=O)Nc2cc(CN3CCNCC3)cc(-c3nc4ncccc4o3)c2)cc1OC] | 159 |
| 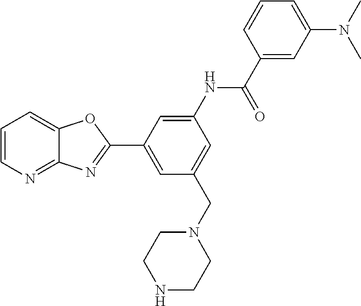 [SMILES: CN(C)c1cccc(C(=O)Nc2cc(CN3CCNCC3)cc(-c3nc4ncccc4o3)c2)c1] | 160 |

### III. COMPOSITIONS

Compositions are provided that include one or more of the agent that increases SIRT1 activity or expression, such as a SIRT1 activator or a nucleic acid encoding a SIRTUIN protein (e.g., SIRT1), or the agent including an expression vector encoding a mutated Nav1.5 protein that is non-acetylatable on lysine residue 1479, that are disclosed herein in a carrier. The compositions can be prepared in unit dosage forms for administration to a subject. The amount and timing of administration are at the discretion of the treating physician to achieve the desired purposes. The agents can be formulated for systemic or local administration. In one example, the agent that increases SIRT1 activity or expression, such as a SIRT1 activator or a nucleic acid encoding a SIRTUIN protein (e.g., SIRT1), is formulated for parenteral administration, such as intravenous administration.

The compositions for administration can include a solution of the agent that increases SIRT1 activity or expression, such as a SIRT1 activator or a nucleic acid encoding a SIRTUIN protein (e.g., SIRT1), or the agent including an expression vector encoding a mutated Nav1.5 protein that is non-acetylatable on lysine residue 1479, dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of agent in these formulations can vary widely, and will be selected primarily based on fluid volumes, viscosities, body weight and the like in accordance with the particular mode of administration selected and the subject's needs. Actual methods for preparing administrable compositions will be known or apparent to those skilled in the art and are described in more detail in such publications as Remington's Pharmaceutical Science, 19th ed., Mack Publishing Company, Easton, Pa. (1995).

An agent that increases SIRT1 activity or expression, such as a SIRT1 activator or a nucleic acid encoding a SIRTUIN protein (e.g., SIRT1), or the agent including an expression vector encoding a mutated Nav1.5 protein that is non-acetylatable on lysine residue 1479, may be provided in lyophilized form and rehydrated with sterile water before administration, although they are also provided in sterile solutions of known concentration. In one example, the agent that increases SIRT1 activity or expression, such as a SIRT1 activator or a nucleic acid encoding a SIRTUIN protein (e.g., SIRT1), or the agent including an expression vector encoding a mutated Nav1.5 protein that is non-acetylatable on lysine residue 1479, can be administered by slow infusion, rather than in an intravenous push or bolus. In one example, a higher loading dose is administered, with subsequent, maintenance doses being administered at a lower level.

Single or multiple administrations of the compositions including the one or more of the agent that increases SIRT1 activity or expression, such as a SIRT1 activator or a nucleic acid encoding a SIRTUIN protein (e.g., SIRT1), or the agent including an expression vector encoding a mutated Nav1.5 protein that is non-acetylatable on lysine residue 1479, are administered depending on the dosage and frequency as required and tolerated by the patient. In any event, the composition should provide a sufficient quantity of at least one of the one or more of the agent that increases SIRT1 activity or expression, such as a SIRT1 activator or a nucleic acid encoding a SIRTUIN protein (e.g., SIRT1), or the agent including an expression vector encoding a mutated Nav1.5 protein that is non-acetylatable on lysine residue 1479 to effectively treat the patient. The dosage can be administered once, but may be applied periodically until either a therapeutic result is achieved or until side effects warrant discontinuation of therapy. The subject can be treated at regular intervals, such as monthly, until a desired therapeutic result is achieved. Generally, the dose is sufficient to treat or ameliorate symptoms or signs of disease without producing unacceptable toxicity to the patient.

Controlled-release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, Pa., (1995). Particulate systems include microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, J., Colloidal Drug Delivery Systems, J. Kreuter, ed., Marcel Dekker, Inc., New York, N.Y., pp. 219-342 (1994); and Tice & Tabibi, Treatise on Controlled Drug Delivery, A. Kydonieus, ed., Marcel Dekker, Inc. New York, N.Y., pp. 315-339, (1992).

Polymers can be used for ion-controlled release of the compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa. (1993)).

The pharmaceutical compositions of the disclosure typically are sterile and stable under conditions of manufacture, storage and use. Sterile solutions can be prepared by incorporating the conjugate in the required amount in an appropriate solvent with one or a combination of ingredients enumerated herein, as required, followed by filtered sterilization. Generally, dispersions are prepared by incorporating the disclosed antigen and/or other biologically active agent into a sterile vehicle that contains a basic dispersion medium and the required other ingredients from those enumerated herein. In the case of sterile powders, methods of preparation include vacuum drying and freeze-drying which yields a powder of the disclosed antigen plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

### EXAMPLES

The following examples are provided to illustrate certain particular features and/or embodiments and should not be construed as limiting.

### Example 1

### SIRT1 Increases the Nav1.5 Sodium Current

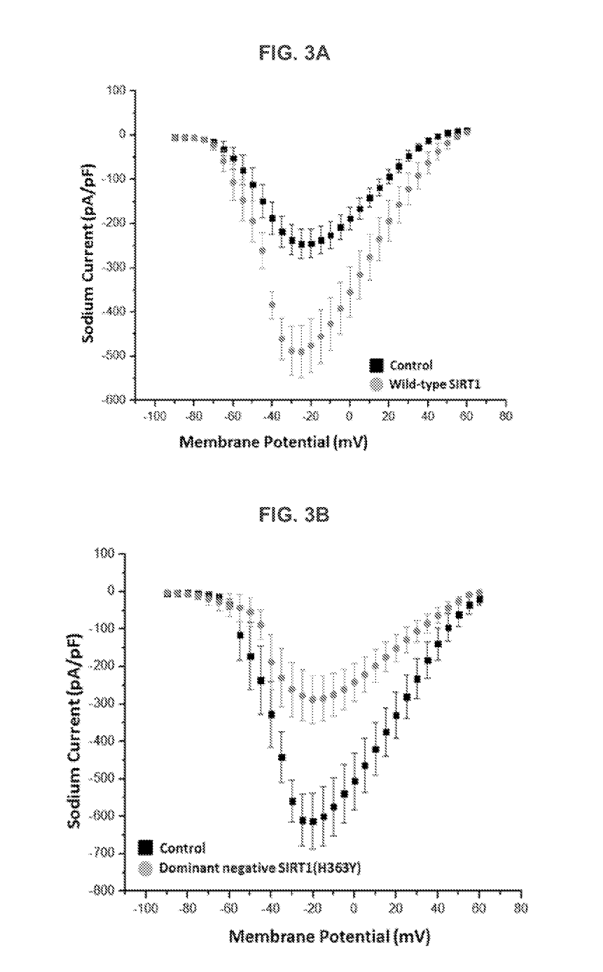

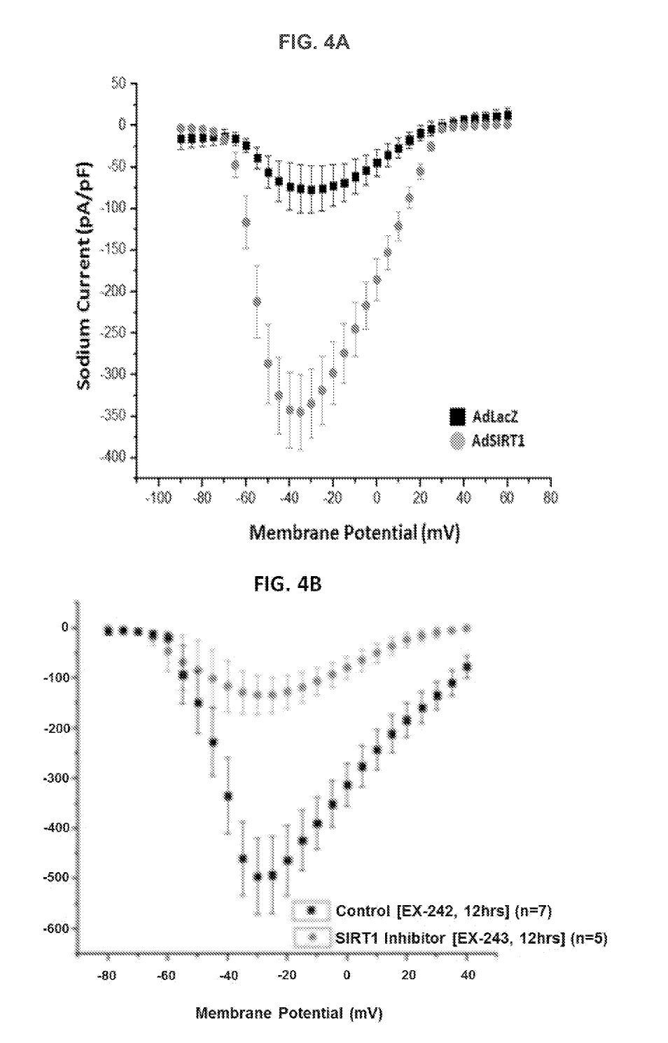

To determine if SIRT1 affects Nav1.5 sodium currents (I_{Na}) I_{Na} was measured by whole cell patch clamp in HEK 293 cells constitutively expressing Nav1.5. Overexpression of wild-type SIRT1 increased I_{Na} (FIG. 3A), while inhibition of endogenous SIRT1 by expression of a catalytically inactive dominant negative mutant of SIRT1, (H363Y), decreased I_{Na} (FIG. 3B). Neither wild-type nor SIRT1 (H363Y) altered other kinetic properties of the channel. It was then showed that adenoviral overexpression of SIRT1 also increased INa in neonatal rat cardiac myocytes (NRCMs) expressing native Nav1.5 (FIG. 4A). Importantly, application of Ex-243, a chemical shown to potently and selectively inhibit SIRT1 (see FIG. 4B), significantly decreased INa in NRCMs compared to its inactive isomer Ex-242 (FIG. 4B). In contrast, experiments showed that SIRT1 overexpression did not alter the K+ current IKr in HEK 293 cells overexpressing HERG (55±2 vs. 55±10 pA/pF, n=3 each, measured from tail currents following maximal activation). Taken together, these results show that SIRT1 regulates Nav1.5 and cardiac INa.

### Example 2

### SIRT1 does not does not Change Expression of Nav1.5

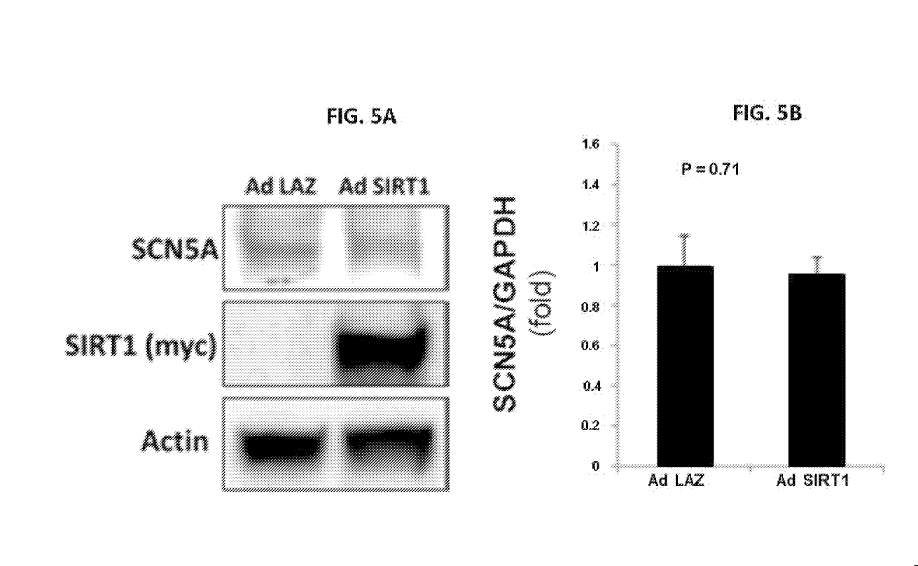

It was then determined if increase in Na current by SIRT1 could be due to an increase in Nav1.5 expression. SIRT1 overexpression in rat neonatal cardiac myocytes did not change expression of Nav1.5 in HEK cells or endogenous Nav1.5 in rat neonatal myocytes either at the protein or mRNA level (FIG. 5).

### Example 3

### SIRT1 Promotes Plasma Membrane Localization of Nav1.5

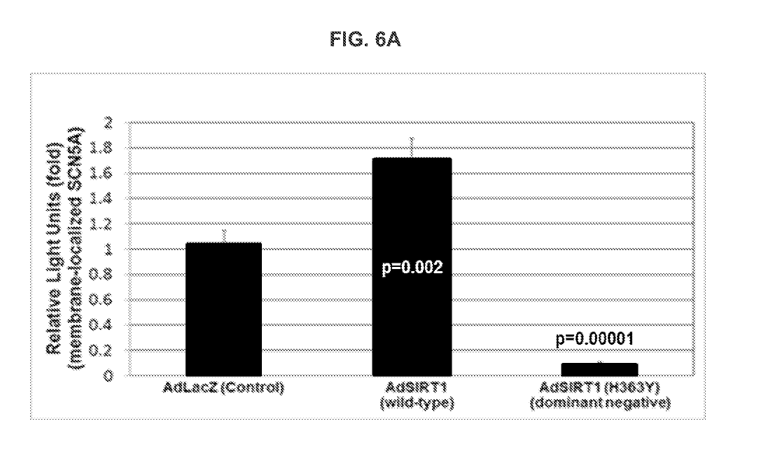

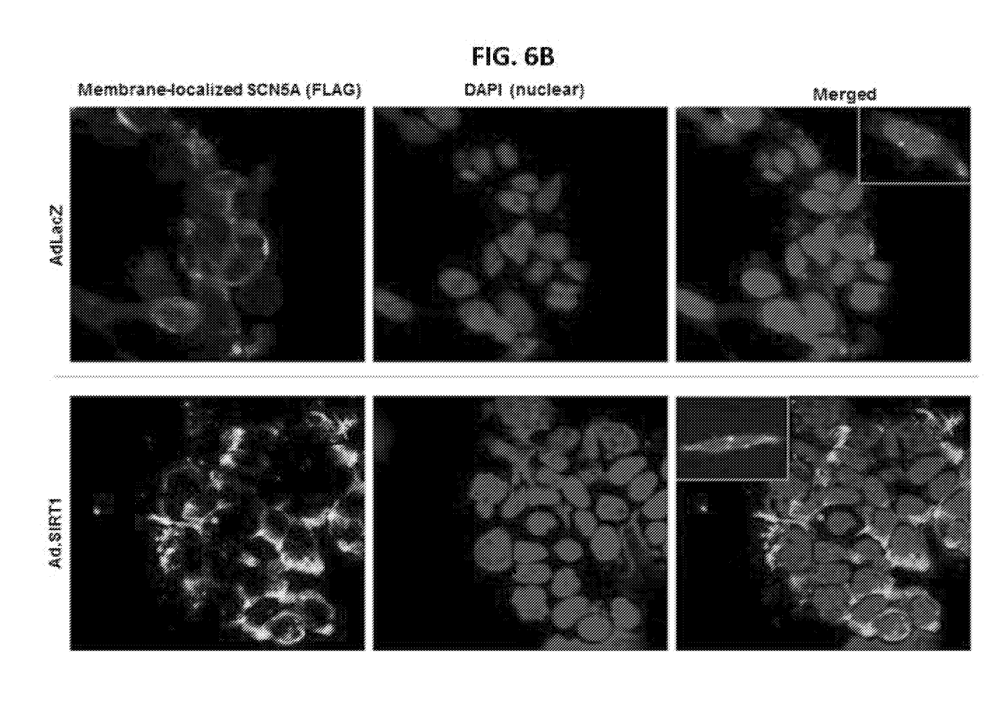

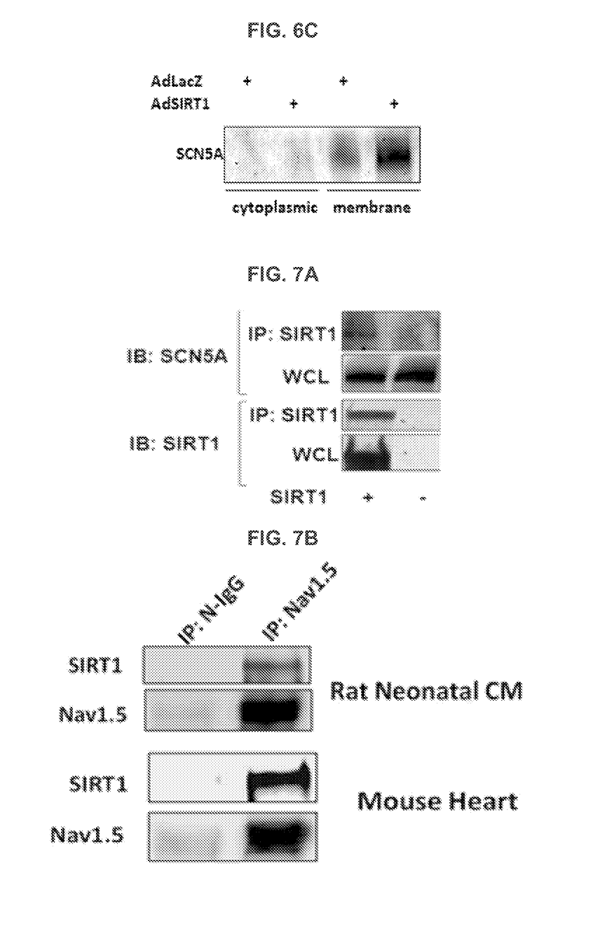

The regulation of total sodium current in a cell is governed by the formula Iₜₒₜ=iNPₒ where i=single channel conductance Pₒ=open probability and N=number of channels in membrane. The whole cell patch clamp data suggests that the change in I_{Na} elicited by SIRT1 is likely due to an alteration in the number of membrane Nav1.5 channels. To determine whether SIRT1 affects plasma membrane localization of Nav1.5, an Nav1.5 construct that contains a FLAG tag in the S5-S6 extracellular loop of domain 1 was developed and used to make a stable HEK 293 cell line. Using this cell line and an immuno-luminescence assay, Nav1.5 channels resident only on the plasma membrane were measured. This assay demonstrated that SIRT1 increases and SIRT1 (H363Y) decreases membrane Nav1.5 channels (FIG. 6A). This effect of SIRT1 on Nav1.5 membrane localization was corroborated by immune-fluorescence experiments (FIG. 6B). In addition, cell fractionation experiments showed that adenoviral overexpression of SIRT1 increases membrane-localized native Nav1.5 in rat neonatal cardiomyocytes (FIG. 6C). These findings strongly suggest that the increase in I_{Na} induced by SIRT1 results from increased membrane expression of the channel.

### Example 4

### Nav1.5 is Acetylated at Lysine Residues and SIRT1 Deacetylates Nav1.5

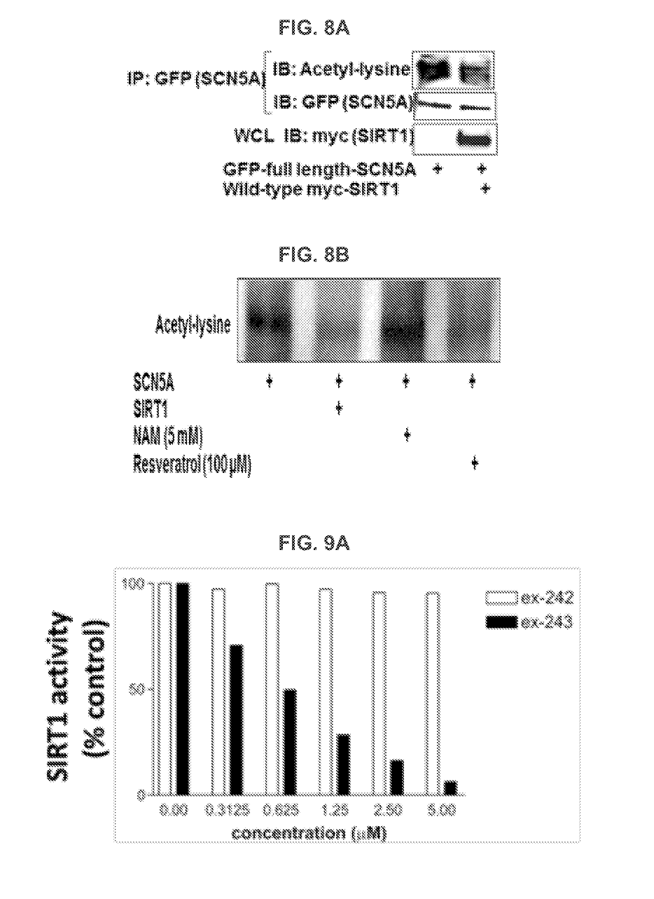

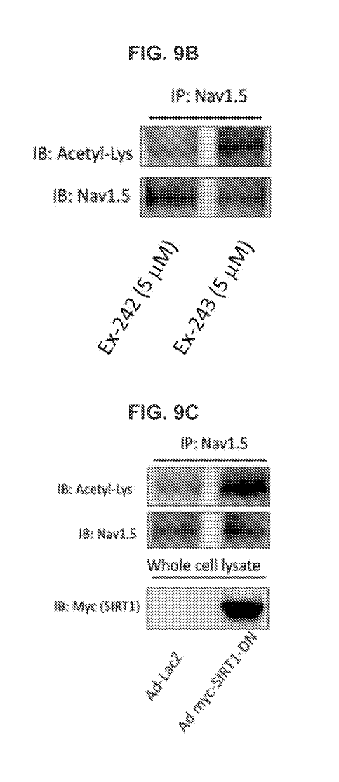

How SIRT1 acts to increase Nav1.5 membrane localization and I_{Na} was addressed. Because SIRT1 is a lysine deacetylase, it was hypothesized that it changes the acetylation status of lysine residues in Nav1.5. To test this hypothesis, whether the two proteins bind to each other was tested. In HEK 293 cells heterologously expressing SIRT1 and Nav1.5, immunoprecipitation of one co-precipitated the other (FIG. 7A), indicating that there is a physical association between the two. In addition, endogenous SIRT1 co-precipitated with endogenous Nav1.5 in NRCMs and in mouse hearts (FIG. 7B). Next, if Nav1.5 is acetylated on lysine residues was determined. In HEK 293 cells expressing the full-length channel, Nav1.5 was robustly acetylated on lysine residues, and overexpression or stimulation (with resveratrol) of SIRT1 decreased, while inhibition of SIRT1 with NAM increased, this acetylation (FIG. 8). In addition, native Nav1.5 in NRCMs was acetylated on lysine residues and inhibition of endogenous SIRT1 either with Ex-243 or by expression of dominant negative SIRT1 increased this acetylation (FIG. 9). Similarly, lysine acetylation of native Nav1.5 was significantly increased in hearts of mice with cardiac-specific knockout of SIRT1 (cSIRT−/− mice, see FIG. 14B).

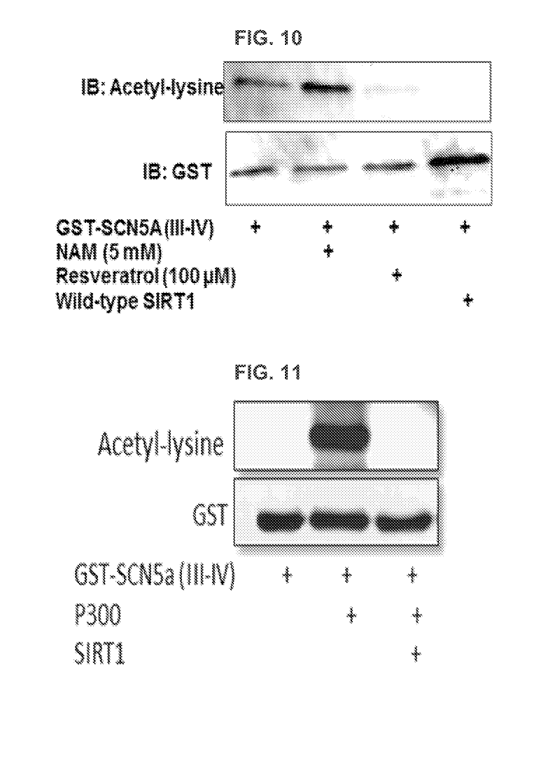

Because Nav1.5 has 84 lysine residues, the number of putative lysine targets of SIRT1 was narrowed down by excluding the extra-cellular and membrane-spanning domains. Attention was initially focused on one intracellular region (termed loop III-IV) which is rich in lysine residues (12 lysines), based on by prior reports showing that lysines in this region are important for channel function (Grant et al., J. Clinic. Invest. 110, 1201-1209, 2002). Using a GST-tagged construct encoding the 61 amino acids in loop III-IV expressed in HEK 293 cells, if lysine acetylation of this region of Nav1.5 is regulated by SIRT1 was examined. Under basal conditions, Nav1.5 (III-IV) was lysine acetylated similar to the full length channel, while inhibition of SIRT1 with nicotinamide (NAM) increased acetylation of Nav1.5 (III-IV) and activation of SIRT1 with resveratrol or overexpression of SIRT1 decreased acetylation (FIG. 10). Taken together, these findings show that SIRT1 binds to and deacetylates Nav1.5, and suggests that lysines in the III-IV intracellular loop of Nav1.5 may be important targets of SIRT1.

### Example 5

### Nav1.5 is a Direct Substrate of SIRT1

Using again GST-Nav1.5 (III-IV) whether this intracellular region of Nav1.5 is a direct target of SIRT1 was next examined. GST-Nav1.5 (III-IV) was acetylated in vitro by the p300 acetyltransferase. In the presence of SIRT1, acetylated GST-Nav1.5 (III-IV) was deacetylated (FIG. 11), indicating that one or more lysines in this region of Nav1.5 are a direct substrate of SIRT1.

### Example 6

### Lysine 1479 in Loop III-IV of Nav1.5 is Targeted by SIRT1

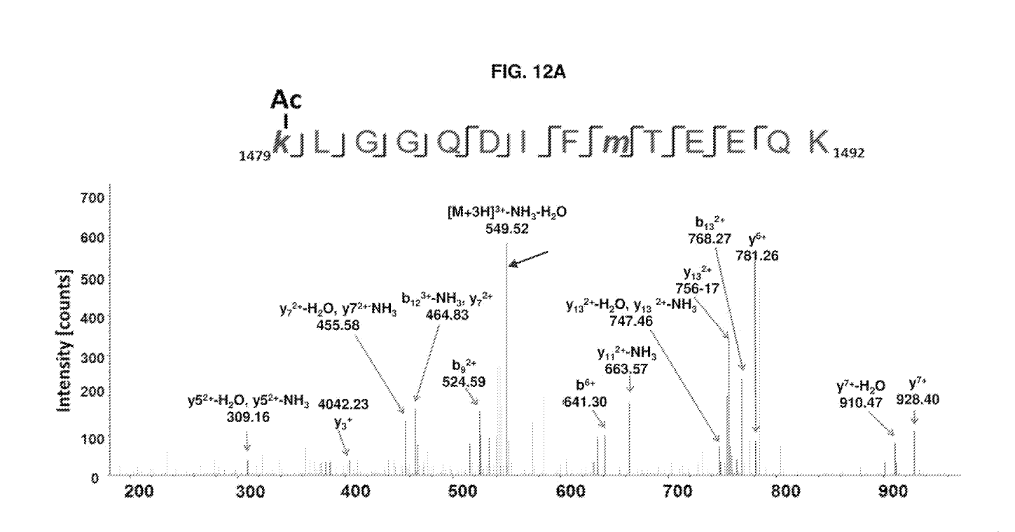

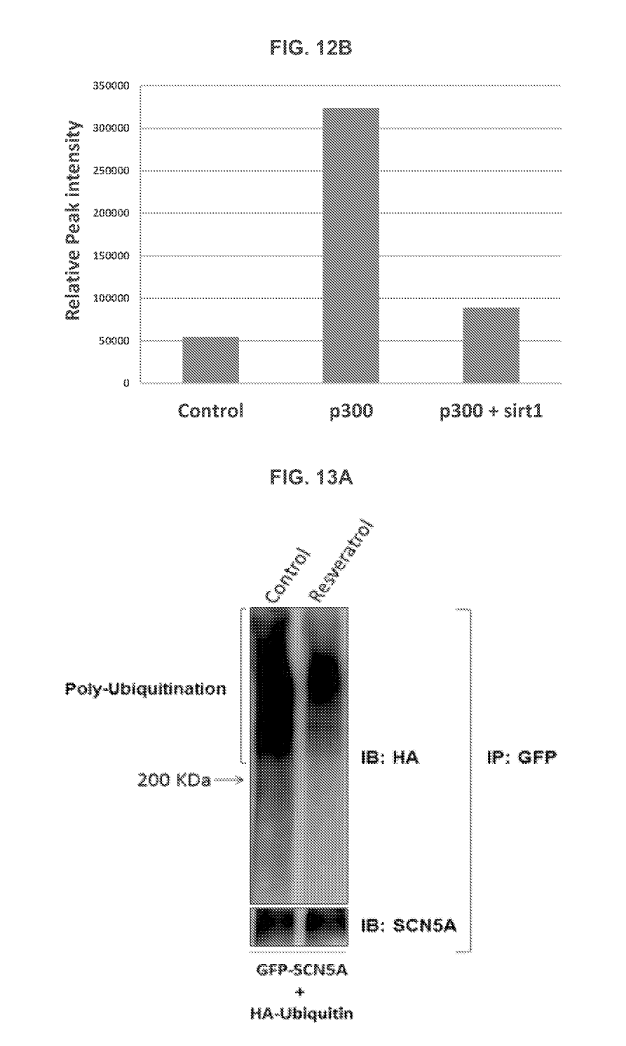

Using GST-Nav1.5 (III-IV) and LC-MS/MS the peptide ¹⁴⁷⁹KLGGQDIFMTEEQK₁₄₉₂ (SEQ ID NO: 9) was identified as containing an acetylated lysine at position 1479 of the full length protein (FIG. 12A). Furthermore, MS-analysis confirmed that this acetylated peptide was almost absent in control and SIRT1-treated GST-Nav1.5 (III-IV) (FIG. 12B) strongly suggesting that lysine 1479 in the III-IV loop of Nav1.5 is targeted for deacetylation by SIRT1.

### Example 7

### SIRT1 Decreases Ubiquitination of Nav1.5

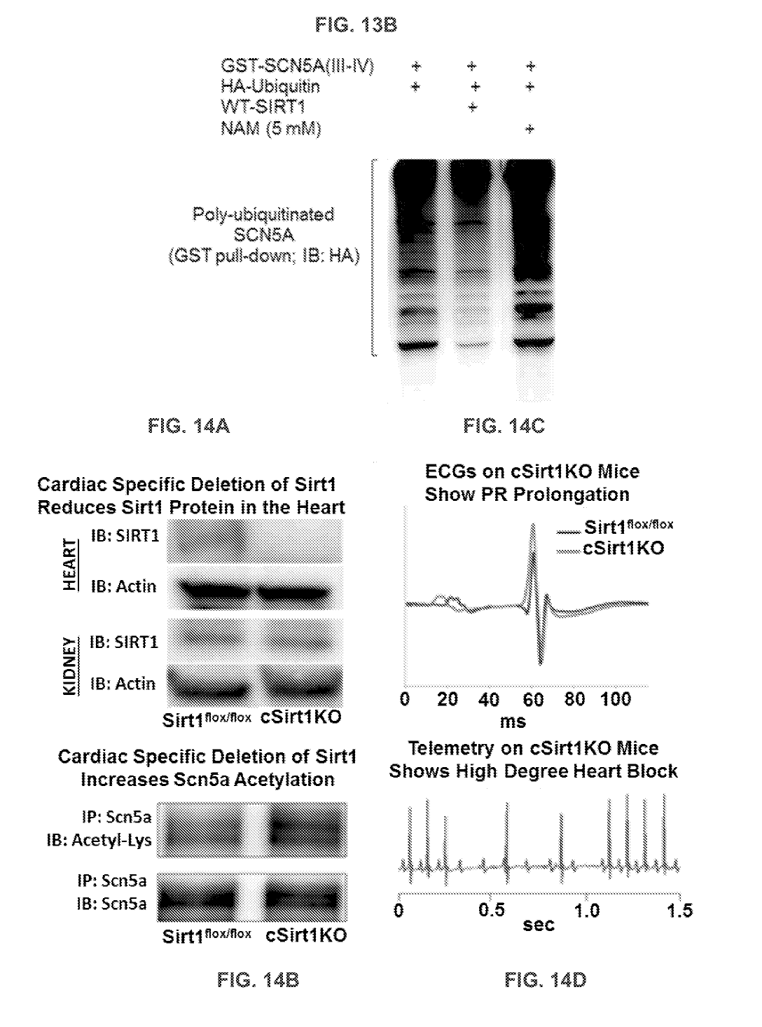

Poly-ubiquitination of Nav1.5 inhibits its membrane localization (Van Bemmelen et al., Circ. Res. 95, 284-291, 2004). SIRT1 regulates ubiquitination of some of its targets, in some cases decreasing ubiquitination (Van Bemmelen et al., Circ. Res. 95, 284-291, 2004). Because SIRT1 increases membrane expression of Nav1.5, whether SIRT1 decreases ubiquitination of the channel was tested. First, the effect of the SIRT1 activator resveratrol on ubiquitination of Nav1.5 was determined. In HEK 293 cells GFP-tagged full-length Nav1.5 was robustly poly-ubiquitinated (FIG. 13A). Moreover, resveratrol significantly decreased this ubiquitination (FIG. 13B). Using GST-Nav1.5 (III-IV) the effect of SIRT1 on ubiquitination of this intracellular region of Nav1.5 was also examined. Similar to full-length Nav1.5, GST-Nav1.5 (III-IV) was also poly-ubiquitinated (FIG. 13B). SIRT1 overexpression decreased this ubiquitination, while inhibition of SIRT1 with NAM increased it (FIG. 13B). These findings suggest a role for de-ubiquitination in mediating the effect of SIRT1 on targeting Nav1.5 to the plasma membrane.

### Example 8

### Mice with Cardiac-Specific Knockout of SIRT1 have Conduction System Abnormalities

To explore the in vivo physiological significance of the above findings, mice with cardiomyocyte-specific knockout of SIRT1 (cSIRT1^{-/-}) were generated using SIRT lflox/flox and αMHC-Cre mice. cSIRT1^{-/-} mice had marked decreases of both SIRT1 normalized mRNA (real-time PCR, 0.12±0.06 heart, 1.02±0.12 kidney, n=2 hearts each) and protein (FIG. 14A) in the heart but not in the kidney. Deletion of SIRT1 increased Nav1.5 acetylation (FIG. 14B). ECG recordings in anesthetized 3-month old cSIRT1^{-/-} mice showed significant prolongation of the PR interval compared to control SIRT1flox/flox mice (43±3 ms, n=4 vs. 37±2 ms, n=5, p=0.01; FIG. 14C) and a trend towards QRS prolongation (15±2 ms, n=4 vs. 13±1 ms, n=5, p=0.23). In addition, continuous ambulatory telemetry monitoring in the cSIRT1^{-/-} mice showed the presence of intermittent high-grade AV block not seen in controls (FIG. 14D). Thus, cardiomyocyte-specific knockout of SIRT1 leads to significant conduction system disease.

### Example 9

### GPD1-L Binds to SIRT1 and Inhibits its Activity

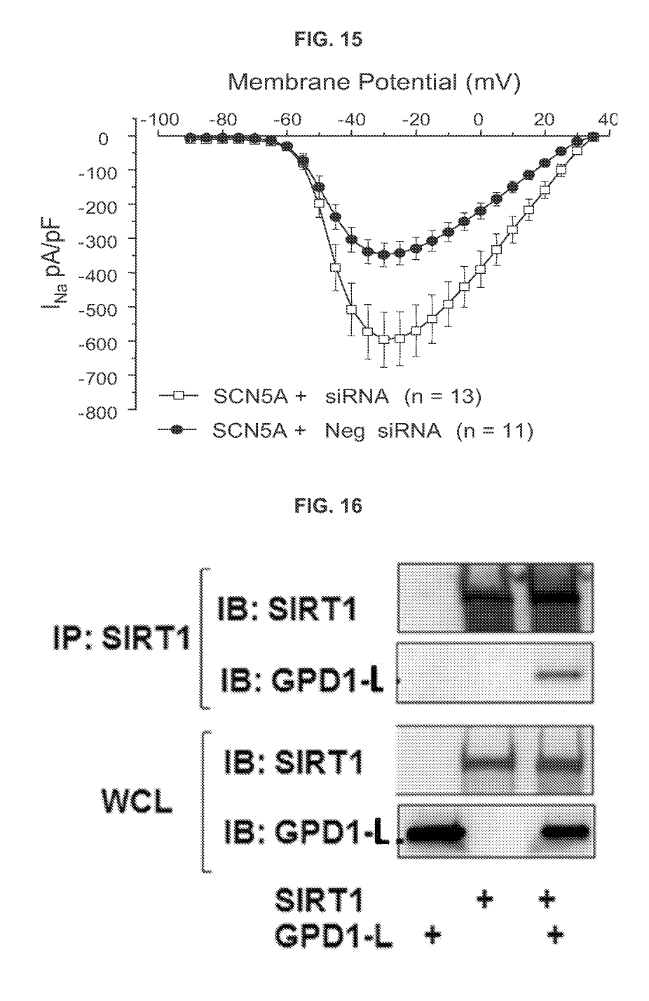

A mutation has been identified in the gene for GPD1-L protein (A280V) in a multigenerational family affected by Brugada syndrome (London et al., Circulation, 116, 2260-2268, 2007). GPD1-L (A280V) decreases the membrane localization of Nav1.5 and I_{Na}(14). It has been postulated that changes in cellular NAD⁺/NADH resulting from decreased GPD1-L enzyme activity in the mutant are responsible for the phenotype (London et al., Circulation, 116, 2260-2268, 2007; Liu et al., Circ. Res., 105, 737-745, 2009; Valdivia et al., Am J Physiol Heart Circ Physiol, 297, H1446-1452, 2009). It is shown herein, however, that mutations in the NAD binding site or in the enzyme's catalytic site that eliminated enzymatic activity increased I_{Na} in Nav1.5-expressing HEK 293 cells; similarly, knockdown of endogenous GPD1-L using siRNA increased I_{Na} (FIG. 15). Thus, GPD1-L is a negative regulator of membrane Nav1.5 membrane expression.

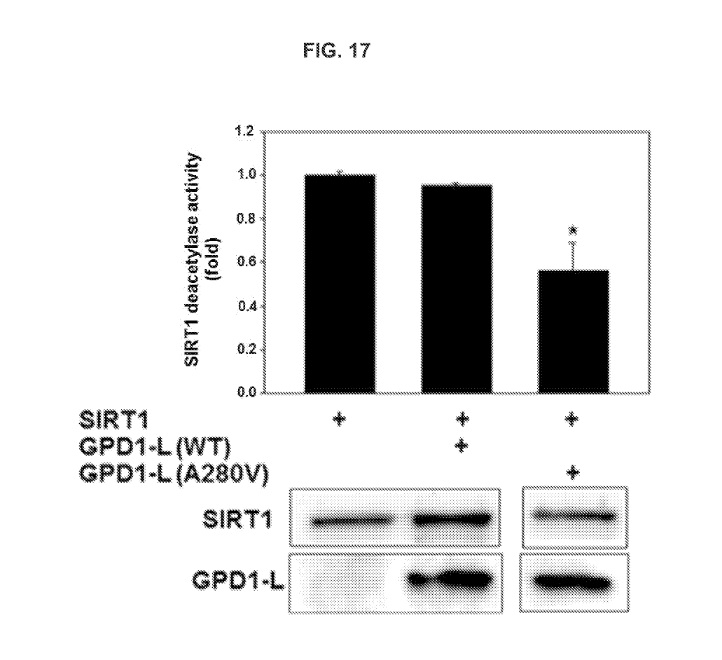

Recognizing that SIRT1 is a NAD⁺-dependent enzyme, whether this mutation in GPD1-L affects I_{Na} via SIRT1 was tested. Whether GPD1-L binds to SIRT1 was first examined. In a heterologous HEK 293 expression system GPD1-L co-precipitated with SIRT1 (FIG. 16). Next, the effect of GPD1-L (A280V) on SIRT1 activity was examined. In HEK 293 cells, GPD1-L (A280V) inhibited SIRT1 enzymatic activity (FIG. 17). A non-limiting explanation for this finding is that mutations in GPD1-L, by modulating SIRT1 activity, impact I_{Na}.

### Example 10

### Generation of Mice with KO and Cardiac-Specific KO of GPDL-L

To explore the in vivo physiological role of GPD1-L and the mechanisms by which mutations may cause decreased I_{Na} and Brugada syndrome, global and cardiac-specific knockout GPD1-L mice were generated. A targeting construct was engineered with a FRP sites flanking the neo cassette and loxP sites flanking exon 2, targeted ES cells were isolated, and chimeras obtained by blastocyst injection. Mating of a male chimera with Cre-expressing females has yielded heterozygous constitutive knockout mice (GPD1L+/− mice), but the knockout was homozygous embryonic lethal (2 litters). Mating of the chimeras with Flp recombinase expressing deleter mice has yielded viable homozygous floxed mice (GPD1Lflox/flox mice), which can be mated with mice transgenic for the Cre recombinase driven by the cardiac-specific α-MHC promoter to generate cardiac specific KO mice (cGPD1L−/− mice).

### Example 11

### Generation of iPS Cells from Brugada-Syndrome Patients with the A280V GPD1-L Mutation

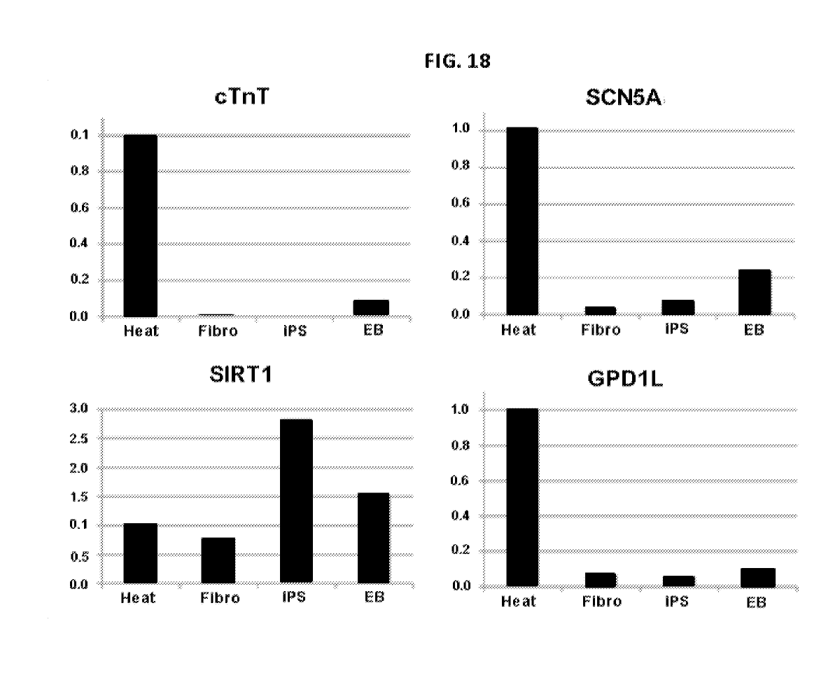

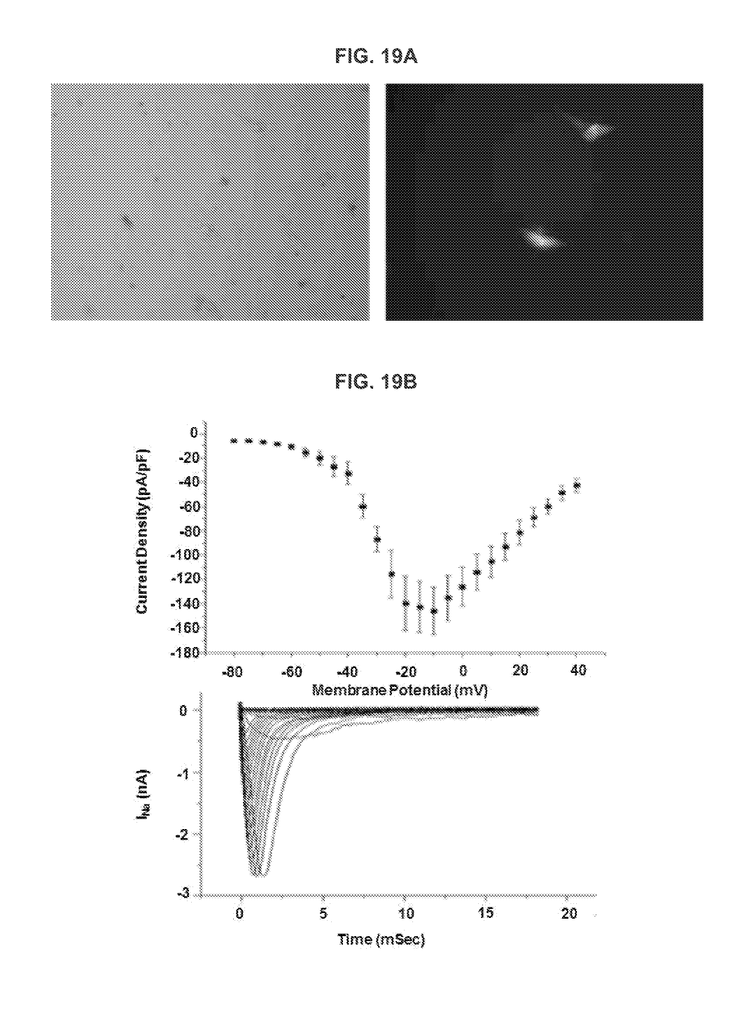

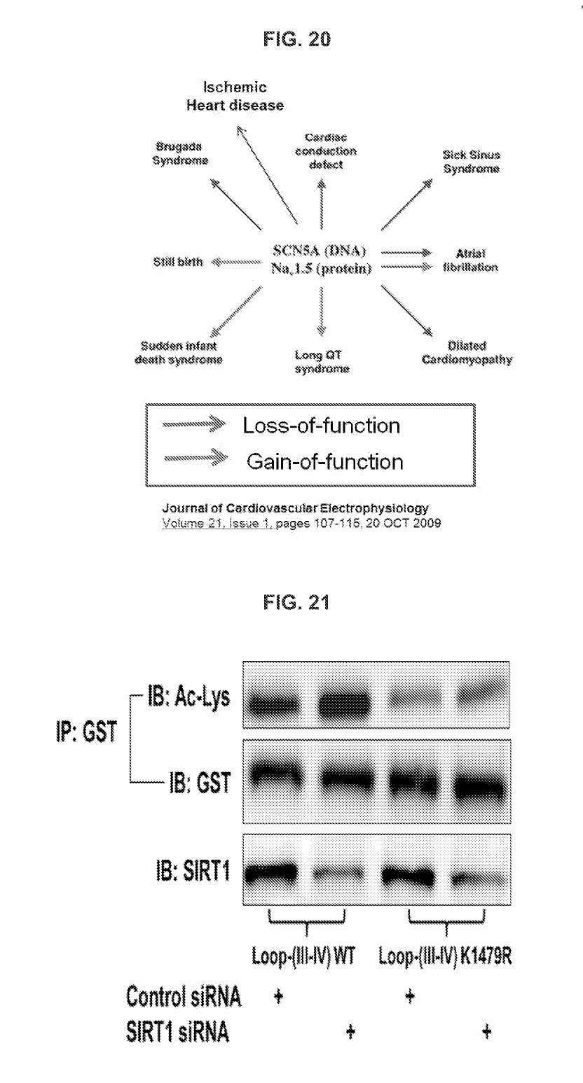

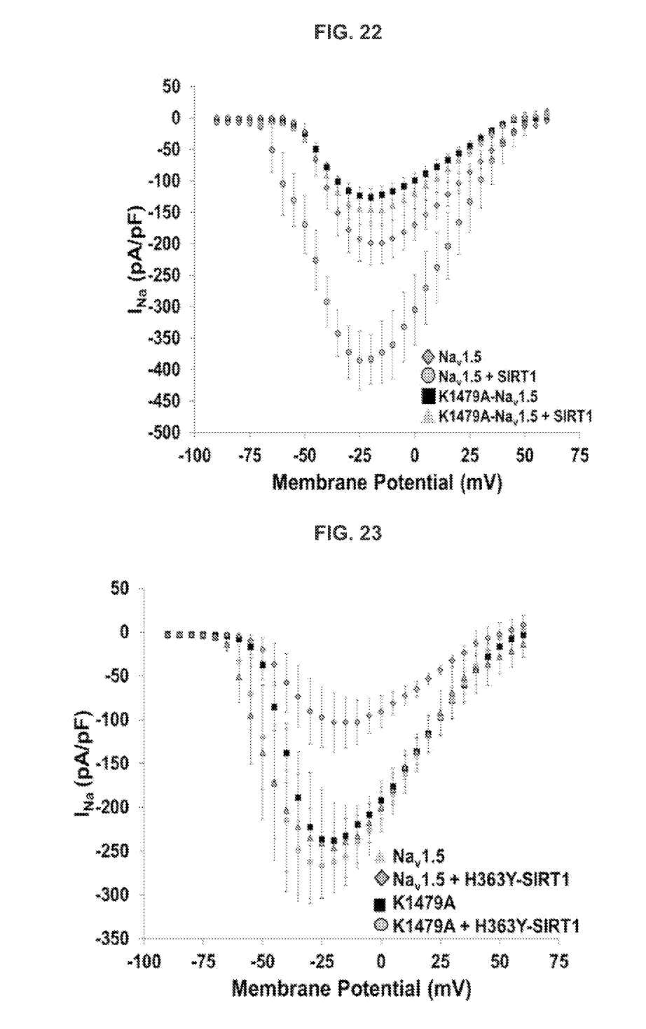

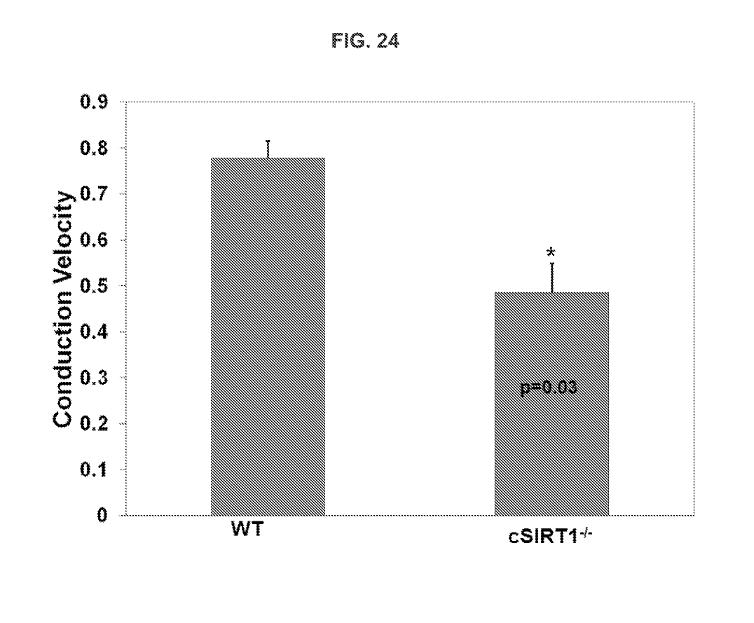

Skin fibroblasts from the proband and a genotypically and phenotypically affected son of the proband of the family affected by Brugada Syndrome with the A280V GPD1-L mutation were isolated. Fibroblasts from the proband of a family with the T353I Nav1.5 mutation were also isolated (Pfahnl et al., Mol Cell., 42, 210-223, 2011). Cell lines of induced pluripotent stem (iPS) cells from each subject have been isolated, and demonstrated (teratomas) that the cells are pluripotent. Differentiation of these cells generated embryoid bodies (EBs) with regions that contracted spontaneously and expressed markers of cardiomyocyte lineage including cTnT (FIG. 18). The EBs express significant amounts of SIRT1, Nav1.5, and GPD1-L, suggesting that they are suitable for the studies outlined below (FIG. 18). Expression levels of these mRNAs did not differ in EBs generated from A280V GPD1-L iPS cells. Only a small fraction of the cells in EB are iPS-derived cardiomyocytes (iPS-CMs). Differentiated iPS cells were infected with AAV6-cTnT-GFP to specifically label iPS-derived cardiac myocytes for patch clamp studies (FIG. 19A), and recorded INa from wild-type iPS-derived cardiac myocytes (FIG. 19B).

It will be apparent that the precise details of the methods or compositions described may be varied or modified without departing from the spirit of the described embodiments. We claim all such modifications and variations that fall within the scope and spirit of the claims below.

1. A method for treating an arrhythmia syndrome due to sodium channel deficiency in a subject, comprising:
selecting a subject with the arrhythmia syndrome due to sodium channel deficiency; and
administering to the subject an effective amount of an agent that increases the expression or activity of SIRTUIN protein in the subject,
thereby treating the cardiac arrhythmia due to the decreased sodium channel in the subject.

2. The method of claim 1, wherein the arrhythmia syndrome is Brugada syndrome, inherited conduction disease, inherited heart failure due to sodium channel mutation, acquired nonischemic cardiomyopathy with sodium channel downregulation, or ischemic cardiomyopathy patients with sodium channel downregulation.

3. The method of claim 1, wherein the syndrome is
Brugada syndrome and the subject has tachyarrhythmia;
inherited conduction disease and the subject has bradyarrhythmia;
inherited heart failure due to sodium channel mutation and the subject has tachyarrhythmia or bradyarrhythmia;
acquired nonischemic cardiomyopathy with sodium channel downregulation and the subject has tachyarrhythmia or bradyarrhythmia; or
ischemic cardiomyopathy with sodium channel downregulation and the subject has tachyarrhythmia or bradyarrhythmia.

4. The method of claim 1, wherein the arrhythmia syndrome is Brugada syndrome, comprising
selecting a subject with Brugada syndrome; and
administering to the subject an effective amount of an agent that increases the expression or activity of SIRTUIN in the subject,
thereby treating Brugada syndrome in the subject.

5. The method of claim 1, wherein the agent that increases the expression or activity of SIRTUIN is a SIRT1 activator.

6. The method of claim 5, wherein the SIRT1 activator comprises Structure I: 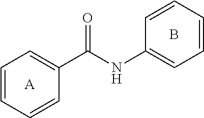
or a salt thereof, wherein:
Ring A is optionally substituted, fused to another ring or both; and
Ring B is substituted with at least one carboxy, substituted or unsubstituted arylcarboxamine, substituted or unsubstituted heteroaryl group, substituted or unsubstituted heterocyclylcarbonylethenyl, or polycyclic aryl group or is fused to an aryl ring and is optionally substituted by one or more additional groups.

7. The method of claim 6, wherein the SIRT1 activator is N-[2-[3-(piperazin-1-ylmethyl)imidazo[2,1-b][1,3]thiazol-6-yl]phenyl]quinoxaline-2-carboxamide:

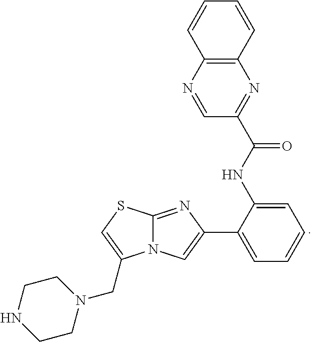 [SMILES: O=C(Nc1ccccc1-c1cn2c(CN3CCNCC3)csc2n1)c1cnc2ccccc2n1]

8. The method of claim 1, wherein the agent increases expression of a SIRTUIN protein.

9. The method of claim 8, wherein the agent comprises a nucleic acid molecule encoding a SIRTUIN protein.

10. The method of claim 9, wherein the SIRTUIN protein is a SIRT1 protein.

11. The method of claim 10, wherein the nucleic acid molecule encodes a polypeptide comprising an amino acid sequence at least 90% identical to the amino acid sequence set forth as SEQ ID NO: 4, SEQ ID NO: 6, or SEQ ID NO: 8, and wherein the SIRT1 protein deacetylates Nav1.5.

12. The method of claim 11, wherein the nucleic acid molecule encodes a polypeptide comprising an amino acid sequence set forth as SEQ ID NO: 4, SEQ ID NO: 6, or SEQ ID NO: 8.

13. The method of claim 9, wherein the nucleic acid molecule encoding the SIRTUIN protein is operably linked to a promoter.

14. The method of claim 13, wherein administering the agent that increases expression of the SIRTUIN protein comprises administering to the subject a vector comprising the nucleic acid molecule encoding the SIRTUIN protein operably linked to the promoter.

15. The method of claim 14, wherein the vector is an adenoviral vector.

16. The method of claim 1, wherein the subject is a human.

17. The method of claim 1, wherein the administration of the agent to the subject increases Nav1.5 activity in cardiac muscle.

18. The method of claim 1, wherein selecting a subject with the arrhythmia syndrome due to sodium channel deficiency comprises selecting a subject with a mutation in the SCN5A gene.

19. The method of claim 1, wherein the subject is a subject with reduced Nav1.5 activity in cardiac muscle.

20-23. (canceled)
